(19) United States
(12) Patent Application Publication
(10) Pub. No.: US 2026/0108591 A1
(43) Pub. Date: Apr. 23, 2026

(54) Malaria pre-erythrocytic antigens as a fusion polypeptide and their use in the elicitation of a protective immune response in a host

(71) Applicant: INSTITUT PASTEUR, PARIS (FR)

(72) Inventors: Rogerio AMINO, BAGNEUX (FR); Pierre CHARNEAU, PARIS (FR); Catherine BLANC, CHANTILLY (FR); Vanessa LAGAL, FONTENAY SOUS BOIS (FR)

(73) Assignee: INSTITUT PASTEUR, PARIS (FR)

(21) Appl. No.: 19/418,144

(22) Filed: Dec. 12, 2025

Related U.S. Application Data
(62) Division of application No. 17/057,095, now Pat. No. 12,521,428.
(60) Provisional application No. 62/675,277, filed on May 23, 2018.

(51) Int. Cl. A61K 39/015; C07K 14/445; C12N 15/86

(52) U.S. Cl. CPC A61K 39/015; C07K 14/445; C12N 15/86; C07K 2319/00; C12N 2740/15034

(57) ABSTRACT

The invention relates to chimeric Plasmodium antigenic polypeptides derived from pre-erythrocytic (PE) antigens and associated in a fusion polypeptide. In particular, the invention relates to antigenic fusion polypeptides of malaria parasites wherein said antigenic polypeptides exhibit a protective effect, especially that of eliciting a protective immune response in a host against challenge by Plasmodium sporozoites or a sterile response. Such identified antigenic fusion polypeptides may thus constitute active ingredients suitable for the design of a vaccine candidate, in particular a vaccine suitable for a human host.

### CROSS REFERENCE TO RELATED APPLICATIONS

This application is a divisional of U.S. application Ser. No. 17/057,095 filed Nov. 19, 2020, which is a U.S. Natl. Stage of International Appln. PCT/IB2019/000702, filed May 23, 2019, which claims the benefit of U.S. application 62/675,277, filed May 23, 2018, all of which are herein incorporated by reference in their entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Dec. 8, 2025, is named DI_2018-26B_SL.xml and is 299,534 bytes in size.

### FIELD OF THE INVENTION

The invention relates to chimeric Plasmodium antigenic polypeptides derived from pre-erythrocytic (PE) antigens and associated in a fusion polypeptide. In particular, the invention relates to antigenic fusion polypeptides of malaria parasites wherein said antigenic polypeptides exhibit a protective effect, especially that of eliciting a protective immune response in a host against challenge by Plasmodium sporozoites or a sterile response. Such identified antigenic fusion polypeptides may thus constitute active ingredients suitable for the design of a vaccine candidate, in particular a vaccine suitable for a human host.

### BACKGROUND OF THE INVENTION

In the last 15 years, malaria control measures reduced by 48% the global deaths caused by this mosquito-borne disease. Despite this significant decrease in mortality, the WHO estimated ˜215 millions of malaria clinical episodes, resulting in more than 400,000 deaths in 2015. Actual malaria control programs rely mainly on the use of insecticides and antiplasmodial medicines, but the emergence and spreading of resistant mosquitos and parasites put the efficacy of these interventions at risk¹. In this scenario, an efficient malaria vaccine could be an important additional tool to control and eventually eliminate malaria.

Since the 60's, it has been known that multiple immunizations using, irradiated sporozoites can elicit sterile protection against malaria infection. However, during the last 50 years only a few protective antigens were identified, but none of them, individually or in combination, could match the robust protection induced by irradiated parasites.

The most advanced malaria vaccine, RTS,S (Mosquirix, GSK), targets the Plasmodium falciparum circumsporozoite protein (CSP), the major surface protein of sporozoites, the motile stage inoculated in the skin during an infective mosquito bite. This subunit vaccine reduced the clinical cases of malaria in African infants and children by 26-36%². This partial protection is mainly associated with high titers of anti-CSP antibodies³, and albeit significant, it is far from achieving the standards established by the WHO malaria vaccine road map, which preconizes the development of a vaccine with at least 75% of efficacy against clinical malaria, and ideally targeting morbidity, mortality and parasite transmission⁴.

On the other hand, live irradiated sporozoites can invade but are arrested as early liver-stages inside hepatocytes, conferring sterile immunity against a homologous sporozoite challenge⁵. Unfortunately, technical and economical impediments associated with the production, storage and delivery of these live parasites still hinder their use for mass vaccination in poor tropical countries. This sterile protection seems to be mainly dependent on CD8+ T cells, since their depletion abolishes sterile immunity in several experimental models, however, the identity of the antigens conferring such robust protection is still elusive⁶. So far, the number of known protective antigens among the thousands of possible proteins expressed by pre-erythrocytic (PE) stages, sporozoite and the ensuing liver-stage, is extremely limited, and these antigens only confer weaker protection than CSP alone or in multi-antigenic formulations in humans⁷. To date the attempts to identify new protective antigens from live attenuated sporozoites have not yielded suitable candidates, despite the screening of thousands of PE peptides, mini-genes and genes⁸⁻¹⁰.

### DESCRIPTION OF THE INVENTION

The invention concerns a chimeric antigen obtained as a fusion polypeptide of distinct antigens and/or antigenic fragments thereof suitable for the elicitation of an immunogenic response, in particular a protective immunogenic response or advantageously a sterile protection against Plasmodium parasite. Such chimeric antigen is suitable as an active ingredient in an immunogenic or in a vaccine composition or as the expression product of an active ingredient (such as a nucleic acid or a lentiviral vector) from which it is expressed.

Sterile protection when assessed in mice model immunized with the antigens, nucleic acids (either with DNA or RNA), or vectors constructs of the invention in particular lentiviral vectors as disclosed herein may be acknowledged if infected red blood cells are not detected after 10 days post inoculation of sporozoites.

Accordingly the invention relates to a chimeric antigenic polypeptide or a nucleic acid encoding such a chimeric antigenic polypeptide or a vector encoding same wherein the chimeric antigenic polypeptide is a fusion of antigens of Plasmodium parasite or antigenic domains or fragments of Plasmodium parasite antigens, in particular of protective domains of such antigens or where the antigens provide at least 2, in particular at least 3 or at least 4 or at least 5 and in particular are especially 2, 3, 4 or 5, and accordingly encompass at least 2, at least 3 or at least 4 antigens (and/or antigenic fragments thereof) or are from the group of antigens (and/or antigenic fragments thereof) designated as 18-10 (ICP), 11-10 (Ag45), TRAP, and 11-09 (Ag40). In a particular embodiment the chimeric antigenic polypeptide (also designated chimeric antigen) comprises or consist of a fusion of the above 4 antigens or antigenic fragments thereof or a combination of antigens within the above group and fragments thereof.

According to another embodiment, the invention relates the chimeric antigenic polypeptide (also designated chimeric antigen) or a nucleic acid encoding such a chimeric antigenic polypeptide or a vector encoding same wherein the chimeric antigenic polypeptide comprises or consists of a fusion of the 5 antigens or antigenic fragments thereof as defined herein wherein such fusion encompasses or consists of antigens or antigenic fragments thereof which are CSP, 18-10 (ICP), 11-10 (Ag45), TRAP, and 11-09 (Ag40).

According to another embodiment said 2, 3, 4 or at least 2, at least 3 or at least 4 antigens or antigenic fragments thereof are comprised in a combination or in a composition of antigens wherein the chimeric antigenic polypeptide is present in the combination or in the composition which additionally comprises as a separate antigen, the CSP antigen or an antigenic fragment thereof of a Plasmodium parasite. In a particular embodiment a chimeric antigenic polypeptide comprising 5 antigens or antigenic fragment(s) of antigens as a fusion polypeptide is provided wherein the CSP or fragment thereof is fused, in particular in the N-terminal end of the chimeric polypeptide.

The invention accordingly also relates to a nucleic acid molecule encoding a chimeric polypeptide defined herein. The nucleic acid may be DNA, in particular cDNA or may be RNA, in particular stabilized RNA. The RNA sequences are deducted from the DNA sequences wherein the Thymine (T) nucleobase is replaced by an Uracile (U) nucleobase. RNA polynucleotides may be obtained by transcription of DNA or cDNA or may be synthesized.

The invention accordingly relates to a combination (i.e., an assembly of separated compounds or alternatively of mixed compounds) of compounds, or a composition of compounds (in admixture), comprising at least 2 distinct active ingredients wherein each active ingredient consists of one of the following types of compound:
(i) an antigenic polypeptide, in particular a chimeric antigenic polypeptide as defined herein, of a Plasmodium parasite,
(ii) a polynucleotide, e.g. a DNA or a RNA, encoding the antigenic polypeptide, in particular the chimeric antigenic polypeptide or,
(iii) a vector, in particular a viral vector, especially a lentiviral vector wherein such vector expresses such antigenic polypeptide of a Plasmodium parasite.

A combination or a composition of compounds comprises or consists in particular of a chimeric antigen defined herein comprising or consisting of at least 2, or at least 3 or at least 4 or at least 5 antigens or antigenic fragment(s) thereof, its nucleic acid or a vector comprising such nucleic acid, especially a viral or a lentiviral vector comprising such nucleic acid in its genome.

In a combination of compounds, additional Plasmodium antigen(s), or antigenic fragments thereof, their nucleic acid or a vector, in particular a viral or lentiviral expressing the same may also be present. Such additional antigenic polypeptides are in particular PE antigens or fragments thereof, such as CSP antigen or a fragment thereof. Such additional antigens, their nucleic acid or a vector, in particular a viral or lentiviral expressing the same, may also be chimeric antigenic polypeptide(s) as defined herein which are different from other chimeric antigenic polypeptides contained in the combination or composition.

In a particular embodiment, the invention accordingly relates to a combination or to a composition of compounds, comprising at least 2 distinct active ingredients wherein each active ingredient consists of an antigenic polypeptide of a Plasmodium parasite, a polynucleotide encoding the antigenic polypeptide, or a vector, in particular a viral vector, especially a lentiviral vector wherein such vector expresses such antigenic polypeptide of a Plasmodium parasite, wherein one antigenic polypeptide is the circumsporozoite protein (CSP) or a polypeptidic derivative thereof.

According to a particular embodiment, a combination or a composition of compounds of the invention thus comprises at least a chimeric antigenic polypeptide which is a fusion of at least 2, in particular at least 3 or at least 4 antigens selected from the group of following antigens: thrombospondin related anonymous protein (TRAP) characterized by the sequence of SEQ ID No. 20, 21, 23, 24, 26 or 27, the inhibitor of cysteine protease (ICP) characterized by the sequence of SEQ ID No. 29, 30, 32, 33, 35 or 36, protein Ag40 (11-09) having one of the sequences of SEQ ID No. 67, 68, 70, 71, 73 or 74 and Ag45 (11-10) having one of the sequences of SEQ ID No. 76, 77, 79, 80, 82 or 83 or independently of each other, a polypeptidic derivatives thereof in particular fragments thereof as defined herein comprising or consisting of the Protective Domain, provided each polypeptidic derivative keeps protective properties of the antigen from which it derives in the combination of compounds.

Optionally circumsporozoite protein (CSP) characterized by the sequence of SEQ ID No. 11, 12, 14, 15, 17, 18 or 124 or a fragment thereof is also present in the chimeric antigen or is also present as an additional separate antigen in the combination or composition of compounds,

A polypeptidic derivative is defined herein as an antigenic fragment of a native antigen. It may alternatively or in combination be defined as a polypeptide whose amino acid sequence consists of an amino acid sequence with at least 70%, in particular at least 86% of identity in amino acids, preferably at least 95% amino acid identity with the antigenic polypeptide from which it derives (especially from P. falciparum or P. vivax) and which keeps the protective properties of the polypeptide from which it derives when it is encompassed in the chimeric antigenic polypeptide or in the composition or combination of compounds of the invention. The threshold of 86% amino acid identity corresponds to the average identity of the three most dissimilar Pf protective antigens (PfCSP; Query cover of 100%, and amino acid identity of 86%) obtained when comparing the 8 P. falciparum pre-erythrocytic antigens of the reference strain known as 3D7 strain (the amino acid sequence of its relevant antigens are those provided herein) with sequences of other P falciparum parasites in the Genbank database identified herein. In a particular embodiment the circumsporozoite protein (CSP) used for determination of the variation threshold is a representative of the worldwide distributed variants of the protein such as CSP VK210 (reference in GenBank: AAKM01000017.1 and protein IDXP_001613068) or CSP VK247 (reference in GenBank: GU339076.1 and Protein ID: ADB92545.1).

### DETAILED DESCRIPTION OF THE INVENTION

The invention thus relates to a chimeric antigenic polypeptide which comprises or consists of a fusion of a fusion of at least2, in particular of 2, 3, or 4 antigens of a Plasmodium parasite infecting human or antigenic fragments:
(a) wherein said antigens are antigens of a Plasmodium parasite, and are selected from the group of antigens designated as TRAP, 18-10 (ICP), 11-10 (Ag45) and 11-09 (Ag40) and,
(b) when the chimeric antigenic polypeptide comprises antigenic fragments of antigens instead of such antigens as defined in (a), such antigenic fragments comprise T cell epitopes in particular CD8+ T cell epitopes and are either truncated antigens or antigenic fragments with amino acid deletion(s) in particular said antigenic fragments contain respectively:
   i. the 18-10 antigen devoid of its signal peptide, and/or or devoid of 1 to 15, in particular 1 to 10 or 1 to 3 amino acid residues,
   ii. the C-terminal fragment of the 11-10 antigen, and/or a fragment thereof deleted from 1 to 3 amino acid residues in its N-terminal end and/or in the C-terminal end,
   iii. the N-terminal fragment of the TRAP antigen, and/or a fragment thereof deleted from 1 to 3 amino acid residues in its N-terminal end,
   iv. a fragment of the 11-09 antigen deleted from 1 to 6 amino acid residues, in particular at its N-terminal end.

The primary structure of the chimeric antigenic polypeptide comprises antigens as defined above and/or antigenic fragments in any order. In a particular embodiment the order of the antigens or antigenic fragments thereof in the fusion may be the following: 18-10 (ICP), 11-10 (Ag45), TRAP and 11-09 (Ag40).

In a particular embodiment, the chimeric antigenic polypeptide does not contain neo-epitopes in the junction of antigen partners in the fusion that would significantly negatively impact the immune response in a host.

In a particular embodiment of the invention, the chimeric antigenic polypeptide above defined comprises or consists of a fusion of antigens or antigenic fragments thereof wherein the antigens and the antigens suitable to provide such fragments are as follows:
a. at least 3, in particular 3 antigens of Plasmodium parasite wherein at least 2 of such antigens are selected from the group of antigens designated as TRAP, 18-10 (ICP), 11-10 (Ag45) and 11-09 (Ag40) and the antigens further include CSP, in particular wherein the antigens include CSP and TRAP or
b. at least 4, in particular 4 antigens of Plasmodium parasite wherein at least 3 of such antigens are selected from the group of antigens designated as TRAP, 18-10 (ICP), 11-10 (Ag45) and 11-09 (Ag40) and the antigens further include CSP, in particular wherein the antigens include CSP and TRAP or
c. at least 5, in particular of 5 antigens of Plasmodium parasite which are TRAP, 18-10 (ICP), 11-10 (Ag45), 11-09 (Ag40) and CSP

In a particular embodiment the CPS antigen is devoid of its signal peptide or is devoid of its GPI fragment or both. In an embodiment, the CSP antigen or an antigenic fragment thereof is fused in the N-terminal end of the chimeric polypeptide. Hence, in an embodiment of the invention, the primary structure of the chimeric antigenic polypeptide results from the junction, in particular a fusion of the following antigens of fragments thereof:
a. at least 2, or at least 3 or at least 4 of the TRAP antigen, the 18-10 antigen devoid of its signal peptide, the C-terminal fragment of the 11-10 antigen, and antigen 11-09 (Ag40) or
b. at least 2, at least 3, at least 4 or at least 5 of the CSP antigen, the TRAP antigen, the 18-10 antigen devoid of its signal peptide, the C-terminal fragment of the 11-10 antigen, and antigen 11-09 (Ag40).

In a particular embodiment, in order to achieve the said junction, in particular to avoid the creation of neo-epitopes that could be significantly recognized by the HLA system in a human host, amino acid residue(s) may be introduced in the chimeric antigenic polypeptide at the junction of two antigens or antigenic fragments thereof such as illustrated in the Example at the junction of antigens/antigenic fragments of 11-10 and TRAP. The number of added residues is preferably minimized and in particular may be 1, 2 or 3 and especially less than 5 contiguous residues.

In a particular embodiment, the chimeric antigenic polypeptide of the invention comprises a fusion of antigenic fragments wherein one fragment is selected from each of each of the following lists:
antigenic fragment of antigen 18-10 wherein the amino acid sequence of such fragment is SEQ ID No. 96 or SEQ ID No. 98 or SEQ ID No. 114 or SEQ ID No; 100 or is a fragment thereof after deletion of 1 to 10, in particular 1 to 3 amino acid residues, especially in the N-terminal end and/or
antigenic fragment of antigen 11-10 wherein the amino acid sequence of such fragment is SEQ ID No. 102 or SEQ ID No. 104 or is a fragment thereof after deletion of 1 to 10, in particular 1 to 3 amino acid residues, especially in the N-terminal end and/or
antigenic fragment of antigen TRAP (11-05) wherein the amino acid sequence of such fragment is SEQ ID No. 106 or SEQ ID No. 108 or is a fragment thereof after deletion of 1 to 10, in particular 1 to 3 amino acid residues, especially in the N-terminal end and/or
antigenic fragment of antigen 11-09 wherein the amino acid sequence of such fragment is SEQ ID No. 110 or SEQ ID No. 112 or is a fragment thereof after deletion of 1 to 10, in particular 1 to 3 amino acid residues, especially in the N-terminal end and/or optionally,
antigenic fragment of antigen CSP wherein the amino acid sequence of such fragment is SEQ ID No. 124 or is a fragment thereof after deletion of 1 to 10, in particular 1 to 3 amino acid residues, especially in the N-terminal end.

In a particular embodiment, the chimeric antigenic polypeptide arising from the fusion of antigenic fragments as disclosed herein is obtained by fusion of antigenic fragments of the same type of Plasmodium parasite such as fragments of Plasmodium falciparum.

As an example, the chimeric antigenic polypeptide is such that its amino acid sequence consists of:
i. the amino acid sequence of SEQ ID No. 96 or SEQ ID No. 98 or SEQ ID No. 100, or SEQ ID No. 114 or the fusion of the amino acid sequences SEQ ID No. 96 and 98 for the 18-10 antigenic fragment fused to,
ii. the amino acid sequence of SEQ ID No. 102 or SEQ ID No. 104 for the 11-10 antigenic fragment fused to,
iii. the amino acid sequence of SEQ ID No. 106 or SEQ ID No. 108 for the TRAP antigenic fragment fused to,
iv. the amino acid sequence of SEQ ID No. 110 or SEQ ID No. 112 for the 11-09 antigen,
   wherein said fragments are devoid or an initial Methionine residue when they feature an internal fusion partner and optionally are devoid of the 1 to 6 of the 1 to 5, in particular the 1 to 3N-terminal residues of the original antigen and further optionally wherein said fragments contain 1 to 3 additional amino acid residues and/or are deleted for 1 to 3 amino acid residues in their N- and/or C-terminal end(s) to enable the junction with the contiguous antigen or antigenic fragment.

In a particular embodiment of this example, the amino acid sequence of the chimeric antigenic polypeptide is such that it:
a. contains 1 to 3 additional junctional amino acid residues at the junction of SEQ ID No. 104 for the 11-10 antigenic fragment and SEQ ID No. 108 for the TRAP antigen, in particular contains one amino acid residue such as the Glutamic acid residue at said junction or,
b. contains a deletion of 1 to 6 or 1 to 5 amino acid residues present in the N-terminal end of the original antigen or antigenic fragment thereof present in the fusion such as the deletion of MA contiguous amino acid residues or MANG contiguous amino acid residues
   it is pointed out that the illustrated amino acid sequences of the antigens, fragments thereof and fusion thereof may contain an initial “M” amino acid residue. Such residue is kept in the used sequence as long as it is necessary for the construct. Accordingly, when the amino acid sequence is contained as an internal fusion partner in the chimeric antigenic polypeptide, the disclosed “M” residue may not be present as an initial residue in the final polypeptide.

In a particular embodiment all the antigens or antigenic fragments thereof provided in the chimeric antigenic polypeptide are from Plasmodium falciparum or from Plasmodium vivax

The invention thus concerns a chimeric antigenic polypeptide whose amino acid sequence is SEQ ID No. 116 or SEQ ID No. 120, or SEQ ID No. 122 (for Plasmodium Berghei) or SEQ ID No. 118 (for Plasmodium falciparum).

The invention also relates to a nucleic acid that encodes a chimeric polypeptide as defined herein. A nucleic acid is thus a DNA, in particular a cDNA, or a RNA. It is the polynucleotide counterpart of the amino acid sequence of the chimeric antigenic polypeptide defined herein and accordingly encodes the same. It may further comprise control nucleotide sequences for the transcription or for the expression of the chimeric antigenic polypeptide. It may also be modified, in order to be operably ligated to a distinct polynucleotide such as a plasmid or a vector genome, in particular a lentiviral vector genome. It may also be modified, in particular to be rendered more stable such as for use as RNA. In a further embodiment, the nucleic acid is a mammalian codon-optimized, in particular a human codon-optimized sequence for expression in mammalian, respectively human cells. The DNA or RNA may be used as such as an active ingredient to elicit an immune response in a host.

In a particular embodiment, the nucleic acid encodes a chimeric antigenic polypeptide of the invention that comprises a fusion of antigenic fragments; accordingly the nucleic acid construct comprises or contains polynucleotide fragments wherein one fragment is selected from each of each of the following lists:
a polynucleotide fragment that encodes an antigenic fragment of antigen 18-10 wherein the amino acid sequence of such fragment is SEQ ID No. 96 or SEQ ID No. 98 or SEQ ID No. 114 or SEQ ID No; 100 or is a fragment thereof after deletion of 1 to 10, in particular 1 to 3 amino acid residues, especially in the N-terminal end, in particular a polynucleotide fragment of SEQ ID No 95. SEQ ID No. 97 or SEQ ID No. 113 and/or
a polynucleotide fragment that encodes an antigenic fragment of antigen 11-10 wherein the amino acid sequence of such fragment is SEQ ID No. 102 or SEQ ID No. 104 or is a fragment thereof after deletion of 1 to 10, in particular 1 to 3 amino acid residues, especially in the N-terminal end in particular a polynucleotide fragment of SEQ ID No 101. SEQ ID No. 103 and/or
a polynucleotide fragment that encodes an antigenic fragment of antigen TRAP (11-05) wherein the amino acid sequence of such fragment is SEQ ID No. 106 or SEQ ID No. 108 or is a fragment thereof after deletion of 1 to 10, in particular 1 to 3 amino acid residues, especially in the N-terminal end in particular a polynucleotide fragment of SEQ ID No 105. SEQ ID No. 107 and/or
a polynucleotide fragment that encodes an antigenic fragment of antigen 11-09 wherein the amino acid sequence of such fragment is SEQ ID No. 110 or SEQ ID No. 112 or is a fragment thereof after deletion of 1 to 10, in particular 1 to 3 amino acid residues, especially in the N-terminal end in particular a polynucleotide fragment of SEQ ID No 109. SEQ ID No. 111 and/or optionally,
a polynucleotide fragment that encodes an antigenic fragment of antigen CSP wherein the amino acid sequence of such fragment is SEQ ID No. 124 or is a fragment thereof after deletion of 1 to 10, in particular 1 to 3 amino acid residues, especially in the N-terminal end in particular a polynucleotide fragment of SEQ ID No 123.

In a particular embodiment all the nucleic acid are derived from nucleotide sequence of Plasmodium falciparum or Plasmodium vivax.

In a particular embodiment of the nucleic acid, the polynucleotide encodes a chimeric antigenic polypeptide whose amino acid sequence consists of:
i. the amino acid sequence of SEQ ID No. 96 or SEQ ID No. 98 or SEQ ID No. 100, or SEQ ID No. 114 or the fusion of the amino acid sequences SEQ ID No. 96 and 98 for the 18-10 antigenic fragment fused to,
ii. the amino acid sequence of SEQ ID No. 102 or SEQ ID No. 104 for the 11-10 antigenic fragment fused to,
iii. the amino acid sequence of SEQ ID No. 106 or SEQ ID No. 108 for the TRAP antigenic fragment fused to,
iv. the amino acid sequence of SEQ ID No. 110 or SEQ ID No. 112 for the 11-09 antigen,

Accordingly, the nucleic acid may be the product of the fusion of the following polynucleotides:
i. sequence of SEQ ID No. 95 or SEQ ID No. 97 or SEQ ID No. 99, or SEQ ID No. 113 or the fusion of the amino acid sequences SEQ ID No. 95 and 97 for the 18-10 antigenic fragment fused to,
ii. the amino acid sequence of SEQ ID No. 101 or SEQ ID No. 103 for the 11-10 antigenic fragment fused to,
iii. the amino acid sequence of SEQ ID No. 105 or SEQ ID No. 107 for the TRAP antigenic fragment fused to,
iv. the amino acid sequence of SEQ ID No. 110 or SEQ ID No. 111 for the 11-09 antigen,

Specific embodiments of the nucleic acid constructs of the invention is one whose nucleotide sequence is SEQ ID No. 115 or SEQ ID No. 119 or SEQ ID No. 121 (for Plasmodium berghei) or SEQ ID No. 117 (for Plasmodium falciparum).

The nucleic acids originating from the Plasmodium parasite may be adapted where necessary to reflect the changes carried out in the original Plasmodium antigen. In particular codon(s) corresponding to the deleted amino acid residues may be deleted from the original sequence or codon(s) encoding the additional amino acid residues may be introduced in the sequence. Additionally the nucleic acid sequence may contain sequences for the control of transcription and/or for the control of expression, and/or may contain sequences for ligation to a distinct nucleic acid such as for ligation to a plasmid or a vector genome. Hence the nucleic acid may contain one or more of sequences for restriction site(s), Kozak sequence, promoter or other sequences as disclosed herein and illustrated in the examples. The particular nucleic acid of the invention may accordingly contain or be devoid of the specific non coding sequences disclosed in the sequences illustrated as SEQ ID No. 84 to 87.

In a particular embodiment the invention relates to the herein defined chimeric antigenic polypeptides provided in a combination or in a composition of compounds wherein said combination or composition further comprises as active ingredients one or more antigenic polypeptide(s) of a Plasmodium parasite which is not contained in the chimeric antigenic polypeptide, or a polynucleotide encoding the antigenic polypeptide(s), or a vector, in particular a viral vector, especially a lentiviral vector, wherein such vector expresses such antigenic polypeptide(s) of a Plasmodium parasite.

In particular, each additional antigenic polypeptide is selected from the group of the circumsporozoite protein (CSP), the metallopeptidase (Bergheilysin/Falcilysin), the GPI-anchored protein P113, the pore-forming like protein SPECT2, or respectively and independently of each other a polypeptidic derivative of any of these antigenic polypeptides wherein said polypeptidic derivative keeps protective properties of the antigen from which it derives in the combination of compounds of the invention.

Accordingly, the combination or composition of compounds including the chimeric antigenic polypeptide defined herein comprises 2, 3, 4, 5, 6, 7 or 8 antigens or polynucleotides encoding such antigenic polypeptides, or a vector, in particular a viral vector, especially lentiviral vector(s), wherein such vector expresses the same or alternatively consists in a combination or a composition of 2, 3, 4, 5, 6, 7 or 8 antigens or viral, especially lentiviral vector(s) expressing the same. In a particular embodiment wherein the combination or composition of compounds comprises a chimeric antigenic polypeptide which is a fusion of 4 antigens or antigenic fragments as defined herein at least 1 antigens or viral vector(s) expressing same is present in the combination or composition and consists of or comprises the circumsporozoite protein (CSP) or a derivative thereof as disclosed herein.

In a particular embodiment of the invention, a combination of compounds is a set of distinct active ingredients present as separate formulation for administration wherein one active ingredient consists of an antigenic polypeptide of a Plasmodium parasite or a polynucleotide encoding this antigenic polypeptide or the active ingredient consists of a vector, in particular a viral vector, especially a lentiviral vector, expressing such antigenic polypeptide of a Plasmodium parasite, wherein said set of active ingredients encompasses chimeric antigenic polypeptides of PE stage antigens of a Plasmodium parasite as defined herein or viral vector, in particular lentiviral vectors expressing such chimeric antigenic polypeptide and other(s) active ingredient(s) consist(s) of other PE stage antigens of a Plasmodium parasite which may be chosen from the circumsporozoite protein (CSP) when it is not present in the chimeric antigenic polypeptide, the metallopeptidase (Bergheilysin/Falcilysin), the GPI-anchored protein P113, the pore-forming like protein SPECT2, or a variant thereof, and a polypeptidic derivative of any of these antigenic polypeptides wherein said polypeptidic derivative keeps protective properties of the antigen from which it derives in the combination of compounds of the invention.

The invention also concerns the active ingredients in admixture in a single composition. Said ingredients whether they are provided for administration as polypeptides (native, recombinant or synthetic), as polynucleotides such as RNA and DNA molecules (modified or not), or as vectors, in particular viral vectors, especially lentiviral vectors capable of expressing said antigenic polypeptides are described as distinct “active ingredients” which means according to the invention, that they individually elicit the immune response against the parasite or that they modulate and in particular broaden and/or enhance qualitatively or quantitatively the immune response which is raised in the host by other antigenic polypeptides in particular by the chimeric antigenic polypeptide provided by or expressed from the combination or the composition of compounds and hence have their own activity or effect on the qualitative and/or quantitative immune response elicited by the combination or composition, in such a way that the combination or composition of compounds elicits a protective response against a Plasmodium infection or against the parasite-induced condition or disease. In addition to being distinct active ingredients, the antigenic polypeptides defined herein are collectively an active ingredient suitable to elicit a protective immune response against a Plasmodium infection or against the parasite-induced condition or disease. The expression “vector” relates to biological or chemical entities suitable for the delivery of the polynucleotides encoding the antigenic polypeptides of the combination of compounds to the cells of the host administered with such vectors. Vectors are well known in the art and may be viral vectors such as adenovirus vectors, especially a vector prepared using Chimpanzee Adenovirus, vector based on pox virus such as MVA-based vectors, canarypox-based vectors, vaccinia-based vectors obtained using modified vaccinia virus, vectors based on Herpes virus such as CMV-based vectors, vesiculovirus-based vectors, measles virus, flavivirus-based vectors or Yellow Fever virus. Vectors obtained from these viruses are disclosed in the art in a way that would enable the person skilled in the art to prepare them. Alternatively and preferably lentivirus vectors are suitable for the preparation of the active ingredients, combination or composition of compounds of the invention, in particular vectors obtained using lentiviruses which infect human, or depending on the host concerned by the protection sought, lentiviruses that infect animals. Examples of such lentiviruses are disclosed herein and the invention relates in particular to the use of HIV vectors, especially HIV-1 vectors. Details for the construction for HIV-1 vectors are provided herein and each disclosed embodiment in this regard is intended to be provided for application with each embodiment relating to the disclosure of the antigenic polypeptides, in particular the chimeric antigenic polypeptides of the invention.

The expressions “Plasmodium parasite” and “malaria parasite” are used interchangeably in the present application. They designate every and all forms of the parasite that are associated with the various stages of the parasite cycle in the mammalian, especially human host, including in particular sporozoites, especially sporozoites inoculated in the host skin and present in the blood flow after inoculation, or sporozoites developing in the hepatocytes (liver-stages), merozoites, including especially merozoites produced in the hepatocytes and merozoites produced in the red-blood cells, or merozoites developing in the red-blood cells (blood-stages). These various forms of the parasite are characterized by multiple specific antigens many of which are well known and identified in the art and some of which are still unknown and to which no biological function has yet been assigned. The antigens can often be designated or classified in groups by reference to their expression according to the stage of the infection. Plasmodium parasites according to the present disclosure encompass parasites infecting human hosts and parasites infecting non-human mammals especially rodents and in particular mice. Accordingly, Plasmodium falciparum, Plasmodium vivax, Plasmodium yoelii and Plasmodium bergheiare particular examples of these parasites. Plasmodium cynomolgi and Plasmodium knowlesi are primarily infectious for macaques, but can also cause human infection. By the expression “antigenic polypeptide”, it is intended according to the present invention a polypeptide which is a chimeric antigenic polypeptide and may be a fusion of native antigens or antigenic fragments thereof of a Plasmodium parasite, or expression product of a gene, codon-optimized or not, of a Plasmodium parasite, in particular of P. berghei, P. cynomolgi or of a Plasmodium parasite infecting humans such as P. falciparum or P. vivax. The application also relates to modified version of such antigenic polypeptides designated as “polypeptidic derivative thereof” used in the chimeric antigenic polypeptide which derivatives can be a fragment of the native antigen of the parasite and especially a truncated version of such native antigen or a fragment obtained by deletion of 1 to 15, in particular 1 to 10 or 1 to 3 amino acid residues of the native antigen (or otherwise designated “original antigen”) or a modified version thereof as a result of post-translational modifications. A derivative polypeptide has an amino acid sequence which is sufficient to provide one or several epitope(s) in particular T cell epitopes and more particularly CD8+ T cell epitopes and which keeps the protective properties leading to the protective activity of the antigenic polypeptide from which it derives and/or exhibits such protective properties when encompassed in the combination or in the composition of compounds of the invention. The protective properties of the reference antigen may even be improved with the derivative. Various examples of derivatives of the antigenic disclosed herein are illustrated in the examples. It may accordingly have a length of at least about 4 amino acid for B epitopes or at least about 8 amino acid residues and in particular from about 8 to about 19 amino acid residues for sequential T epitopes. In a particular embodiment, the recombinant polynucleotide of the lentiviral vector encodes a fragment of an antigen of the malaria parasite, especially a fragment which results from the deletion of contiguous amino acid residues of the full-length (i.e., native) antigen, such as deletion at the junction of the fusion partners for the chimeric antigenic polypeptide provided it keeps the capacity of the native antigen to elicit an immune response in a host. The polypeptidic derivative as defined hereabove should be considered an alternative for the preparation of the chimeric antigenic polypeptide in any definitions or embodiments of the invention unless it appears irrelevant in the context of the disclosure.

The expressions “T-epitope” and “B-epitope” refer to antigenic determinants that are involved respectively in the adaptive immune response driven by T cells and in the immune response driven by B cells. In particular said T-epitopes and respectively B-epitopes elicit T cell, respectively B cell immune response when delivered to the host in suitable conditions. According to a particular embodiment the antigenic polypeptides targeted according to the invention and the polypeptide derivatives of these antigenic polypeptides comprise epitope(s) mediating CD8⁺ T cell response. In a particular embodiment, alternatively or cumulatively, the antigenic polypeptides of the invention and the polypeptide derivatives of these antigenic polypeptides comprise epitope(s) mediating an antibody response. In a preferred embodiment, the chimeric antigenic polypeptide, the combination or the composition of compounds all together comprise or enable expression of T and B epitopes and therefore elicit both cellular and humoral immune response in a host.

In a particular embodiment of the invention, the combination or the composition of compounds comprises, at least one, preferably at least two antigenic polypeptide(s) including at least one chimeric antigenic polypeptide or when provided as a polynucleotide or as a recombinant vector, especially lentiviral vector the combination or the composition of compounds comprises at least one, preferably at least two recombinant polynucleotide(s) which encodes an antigenic polypeptide(s) wherein said antigenic polypeptide(s) is chimeric antigenic polypeptide or encompasses the latter together with the circumsporozoite protein (CSP) (if not present in the chimeric antigenic polypeptide) of a Plasmodium parasite selected from the group of Plasmodium falciparum, Plasmodium malariae, Plasmodium vivax, Plasmodium ovale or Plasmodium knowlesi and Plasmodium berghei, in particular the group of Plasmodium falciparum and Plasmodium vivax. It is especially a truncated version of the CSP and in particular a polypeptide devoid of the GPI anchoring motif of the CSP. In such combination or composition of compounds, additional polypeptide(s) or polynucleotide(s) may be contained in the viral, especially lentiviral vector(s) and they are also selected in the above disclosed groups of Plasmodium parasites.

In a particular embodiment of the active ingredient, the combination or the composition of compounds of the invention are provided as polynucleotides or as vectors, In particular lentiviral vectors expressing antigenic polypeptides are provided wherein the vectors have or comprise in their genome (vector genome) a recombinant polynucleotide which encodes at least a chimeric antigenic polypeptide of Plasmodium berghei as illustrated in the examples or advantageously an orthologous sequence of Plasmodium falciparum, or Plasmodium vivax as disclosed or illustrated herein e.g., a polypeptide corresponding to a fragment of said antigen

In a particular embodiment of the invention, the active ingredient, the combination or the composition of compounds comprises or consists in separate active ingredients or separate compositions of single or of multiple active ingredients. These active ingredients provided as separate compositions or packages in the combination may be used for separate administration to the host or to the contrary for combined administration.

In another particular embodiment of the invention, the combination of compounds comprises or consists in an admixture of all the active ingredients, otherwise stated consists in a single composition of said active ingredient(s).

Accordingly chimeric antigenic polypeptides of the invention may especially be provided as the expression product of a vector, in particular of lentiviral vectors, in particular HIV-1 based vectors, wherein each vector expresses the chimeric antigenic polypeptide. This type of vector may be used alone or may be provided with additional identical or different vectors that express further antigenic polypeptide(s) of Plasmodium parasite, in particular of the same Plasmodium parasite. Thus a collection of vectors may be provided that expresses all the antigenic polypeptides suitable to elicit an immune response when administered in a host in need thereof. This collection of vectors may be provided as a single composition for administration or as separate compositions for administration to the host simultaneously or separately in time.

In another embodiment, the active ingredients are or comprise nucleic acid, in particular DNA or RNA that encodes the chimeric antigenic polypeptides of the invention.

When used as such, the chimeric antigenic polypeptide or their coding nucleic acid, advantageously provided in a vector such as a lentiviral vector as disclosed herein, or alternatively when used together in a combination or composition these antigenic polypeptides, nucleic acids or vectors consisting of the chimeric antigenic polypeptide of the invention or their coding nucleic acid (especially when provided as vectors expressing the same)=constitute active ingredients that may be regarded as suitable for the elicitation of a protective immune response, preferably a sterile protection against stringent challenge of immunized non-human mammal with Plasmodium parasite from which the polypeptides originate. Accordingly the compounds of the invention provide a response to the need for efficient alternative against Plasmodium infection by devising active ingredients which may be used for the elaboration of a vaccine candidate in human host.

Whatever its presentation as one or more compositions, the combination or the composition of compounds of the invention provides individual and collective active ingredients (as antigenic polypeptides or as vector particles especially lentiviral vector particles) which constitute collectively the qualitative composition for a dose of a candidate medicine product.

In a particular embodiment of the combination of compounds or composition of the invention, the active ingredients consist of chimeric antigenic polypeptides of a human-infecting Plasmodium parasite or consist of nucleic acids or of lentiviral vector(s) expressing antigenic polypeptides of a human-infecting Plasmodium parasite, or consist in a mixture or an association of such chimeric antigenic polypeptides with additional antigenic polypeptides of the Plasmodium parasite or nucleic acids or viral vectors expressing the chimeric antigenic polypeptides and possibly additional antigenic polypeptides, especially lentiviral vectors, in particular wherein the Plasmodium parasite is Plasmodium falciparum or Plasmodium vivax.

Specific polypeptidic derivatives disclosed for use according to the invention are in particular obtained by substitution of amino acid residues in the original sequence of the Plasmodium antigen and/or by point mutations (such as substitution, insertion or deletion) or deletion(s) of short sequence(s) in said original sequence, to the extent that the derived polypeptide keeps essentially the immunogenic properties of the polypeptide from which it derives. Derivatives can thus be illustrated by the polypeptides including in their sequence residues originating from the polynucleotide construct from which they are obtained such as amino acid residues resulting from the presence of a Kozak sequence in the polynucleotide. Other derivatives may be obtained by conservative substitution of amino acid residue(s), especially amino acid substitution of less than 20% in particular less than 15% or less than 5%, in particular less than 3% or less than 2% of the original amino acid residues of the sequence of the antigen. Without considering the optional addition of functional amino acid sequence(s) to the natural or mutated ORF (Open Reading Frame) of the antigenic polypeptide, such derivatives obtained by substitution, in particular conservative substitutions of amino acid residues, have in particular the same length as the original sequence from which they derive. Alternatively, when the derivative polypeptide has an ORF which consists in a mutant by deletion or by addition with respect to the original ORF, the length of the mutated ORF determined in respect of the number of amino acid residues in the expressed polypeptide derivative is advantageously at least 95% of the length of the original sequence, preferably at least 97%; 98% or 99% identical to the original sequence.

In a particular embodiment of the active ingredients comprise or consist of human lentiviral vector(s) expressing the chimeric antigenic polypeptides including, in a particular embodiment, when obtained using polypeptidic derivatives of Plasmodium antigens, in particular HIV-1 lentiviral vector(s). In a particular embodiment these human vector(s) expressing the chimeric antigenic polypeptides further express additional antigenic polypeptides of Plasmodium parasite and such vectors are expressed:
either individually from separate vectors and/or,
from one or more vectors wherein at least one vector expresses more than one antigenic polypeptide, including the chimeric antigenic polypeptide.

In a particular embodiment wherein the active ingredients are lentiviral vectors, especially HIV-1 based vectors, each lentiviral vector is a replication-incompetent pseudotyped lentiviral vector, in particular a replication-incompetent pseudotyped HIV-1 lentiviral vector, wherein said vector contains a genome comprising a mammal codon-optimized synthetic nucleic acid, in particular a human-codon optimized synthetic nucleic acid, wherein said synthetic nucleic acid encodes the antigenic polypeptide(s) of a Plasmodium parasite infecting a mammal, in particular a human host, or a polypeptidic derivative thereof. The malaria parasite may be in particular Plasmodium falciparum, Plasmodium vivax, P. knowlesi, P cynomolgi, P malariae, P ovale.

Use of codon-optimized sequences in the genome of the vector particles allows in particular strong expression of the antigenic polypeptide in the cells of the host administered with the vector, especially by improving mRNA stability or reducing secondary structures. In addition the expressed antigenic polypeptide undergoes post translational modifications which are suitable for processing of the antigenic polypeptide in the cells of the host, in particular by modifying translation modification sites (such as glycosylation sites) in the encoded polypeptide. Codon optimization tools are well known in the art, including algorithms and services such as those made available by GeneArt (Life technologies-USA) and DNA2.0 (Menlo Park, California—USA). In a particular embodiment codon-optimization is carried out on the ORF sequence encoding the antigenic polypeptide or its derivative and the optimization is carried out prior to the introduction of the sequence encoding the ORF into the plasmid intended for the preparation of the vector genome. In another embodiment additional sequences of the vector genome are also codon-optimized.

The active ingredients consisting of the viral vectors may be integrative pseudotyped lentiviral vectors, especially replication-incompetent integrative pseudotyped lentiviral vectors, in particular a HIV-1 vector. Such lentiviral vectors may in addition contain a genome comprising a mammal-codon optimized synthetic nucleic acid, in particular a human-codon optimized synthetic nucleic acid, wherein said synthetic nucleic acid encodes the antigenic polypeptide(s) of a Plasmodium parasite infecting a mammal such as disclosed herein, in particular a parasite infecting a human host, or a polypeptidic derivative thereof as disclosed herein.

Alternatively the lentiviral vector and in particular the HIV-1 based vector may be a non-integrative replication-incompetent pseudotyped lentiviral vector.

A particular embodiment of a lentiviral vector suitable to achieve the invention relates to a lentiviral vector whose genome is obtained from the pTRIP vector plasmid wherein the Plasmodium synthetic nucleic acid encoding the antigenic polypeptide or polypeptidic derivative thereof has been cloned under control of a promoter functional in mammalian cells, in particular the human beta-2 microglobulin promoter, and wherein the vector optionally comprises post-transcriptional regulatory element of the woodchuck hepatitis virus (WPRE).

In a further embodiment of the invention, the lentiviral vector expressing the chimeric antigenic polypeptide(s) according to the features herein described is pseudotyped with the glycoprotein G from a Vesicular Stomatitis Virus (V-SVG) of Indiana or of New-Jersey serotype.

The particular features of such lentiviral vectors will be further discussed in detail below.

The chimeric antigenic polypeptides of the invention may advantageously be expressed from nucleic acid molecules characterized by the following sequences, and in particular are expressed from mammalian codon-optimized synthetic nucleic acids: SEQ ID No. 115, SEQ ID No. 117, SEQ ID No. 119, SEQ ID No. 121.

Codon optimization of the polynucleotide may influence post translational modifications of the encoded polypeptide, in particular when it is expressed in mammalian cells and therefore enables the expression of polypeptides which harbor structural features which distinguish over those of the polypeptide encoded by the corresponding non-optimized sequence (Mauro V. P. and Chappell S. A. Trends Mol Med-2014 November; 20(11): 604-613).

Similarly the lentiviral vectors expressing the antigenic polypeptides in the combination of compounds may advantageously contain in their genome nucleic acid molecules which are mammalian codon-optimized synthetic nucleic acids characterized by the following sequences: SEQ ID No. 115, SEQ ID No. 117, SEQ ID No. 119, SEQ ID No. 121. Additionally the nucleic acid of the invention may be a polynucleotide comprising one of the following DNA sequences: SEQ ID No. 115, SEQ ID No. 117, SEQ ID No. 119, SEQ ID No. 121 or with a RNA sequence deducted from such DNA sequence. The invention also relates to a formulation suitable for administration to a mammalian host comprising an active ingredient as defined herein or a combination or a composition of compounds according to any one of the definitions provided herein, as active ingredient for protection against a Plasmodium infection or against the parasite-induced condition or disease, together with excipient(s) suitable for administration to a host in need thereof, in particular a human host.

In another aspect of the invention the active ingredient or the combination or the composition of compounds of the invention or the formulation comprising the same is for use in the protective immunisation against malaria parasite infection or against parasite-induced condition or disease, in a mammalian host, especially a human host, optionally in association with an appropriate delivery vehicle and optionally with an adjuvant component and/or with an immunostimulant component.

Accordingly, the active ingredient, the combination or the composition of compounds, in particular the lentiviral vector particles of the invention, when administered to a host in needs thereof, especially to a mammalian in particular to a human host, elicits an immune response, encompassing activation of naïve lymphocytes and generation of effector T-cell response and generation of immune memory antigen-specific T-cell response against antigen(s) of the malaria parasite. The immune response may additionally involve a humoral response against antigenic polypeptides delivered to or expressed in the host following administration of the combination of compounds. The immune response may either prevent the infection by the malaria parasite when such parasite is inoculated as sporozoite to the host or may prevent the onset or the development of a pathological state resulting from inoculation of malaria parasite in the form of sporozoite or prevent the onset or the development of the consequences of the generation of further forms of said parasite such a merozoite forms.

Accordingly, the active ingredients, the combination or the composition of compounds of the invention are suitable for the elicitation of a protective immune response against the parasite infection or against the parasite-induced disease or condition. Such response enables in particular, control or inhibition of the onset of the pathology caused by inoculation of the parasite or by the induction of the exo-erythrocytic i.e., hepatic, stage of the cycle of the malaria parasite and in an advantageous embodiment this response is suitable to prevent, alleviate or inhibit the onset or development of the erythrocytic stage of said parasite. Advantageously, it has been observed that the active ingredient, the combination or the composition of compounds of the invention especially when the active ingredients are provided as lentiviral vector particles used in a single administration regimen or in a prime-boost regimen of administration enable the development of a protective immunity and especially enable a sterilizing protection against the malaria parasite-induced pathology. Such a sterilizing protection may result from controlling the consequences of the infection at the stage of liver infection, if not before, in the cycle of the parasite.

In a particular embodiment of the invention, the active ingredient, the combination or the composition of compounds, especially when the active ingredients are provided as lentiviral vector(s) is a suspension formulated with a suitable administration vehicle for administration to the host. Physiologically acceptable vehicles may be chosen with respect to the administration route of the immunization composition. In a preferred embodiment administration may be carried out by injection, in particular intramuscularly or, for children by intranasal administration or topical skin application. An active ingredient, a combination or a composition of compounds of the invention is used for protective immunisation against malaria parasite infection or against parasite-induced disease or condition in a mammalian host, especially in a human host, said use involving an immunisation pattern comprising administering an effective amount of the active ingredients to elicit the cellular and/or humoral immune response of the host, possibly as a prime and where appropriate later in time administering an effective amount of said active ingredients to boost the cellular immune response of the host, and optionally repeating (once or several times) said administration step for boosting, wherein if the active ingredients are provided as the lentiviral particles administered in each of the priming or boosting steps they are pseudotyped with distinct pseudotyping envelope protein(s) which do not cross-neutralise with each other, and wherein said priming and boosting steps are separated in time by at least 6 weeks, in particular by at least 8 weeks.

Details on the administration regimen will be discussed further below.

The active ingredient, the combination of the composition of compounds of the invention especially as lentiviral vector is especially used in a particular embodiment for the protective immunization against malaria parasite infection or against parasite-induced pathology in mammalian, host, especially in a human host to obtain at least a cellular immune response (T-cell immune response), particularly a CD8-mediated cellular immune response or a CD4-mediated cellular immune response i.e., an immune response which is mediated by activated cells harbouring CD8 or CD4 receptors, preferably Cytotoxic T lymphocytes (CTL) and memory T cell response are advantageously targeted when defining the immunization regimen of the lentiviral particles of the invention.

The immune response can also involve a humoral response i.e., antibodies, elicited by said compounds, produced against said at least one antigenic polypeptide. In a particular embodiment, said humoral response is a protective humoral response. The protective humoral response results mainly in maturated antibodies, having a high affinity for their antigen, such as IgG or IgM. In a particular aspect, the protective humoral response is T-cell dependent. In a particular embodiment, the protective humoral response induces the production of neutralizing antibodies.

In a particular embodiment of the invention, the active ingredient, the combination or the composition of compounds of the invention especially when the active ingredients are lentiviral vectors, even when used in a form which has defective integrase, is able to elicit an early immune response. The expression “early immune response” refers to a protective immune response (protection against the parasite or against the parasite-induced pathology) that is conferred within about one week after the administration of the product.

In another particularly advantageous embodiment, the immune response conferred by the active ingredient, the combination or the composition of compounds of the invention especially as lentiviral particles is a long-lasting immune response i.e., said immune response encompasses memory cells response and in particular central memory cells response; in a particular embodiment it can be still detected at least several months.

When the immune response includes a humoral response, the long-lasting response can be shown by the detection of specific antibodies, by any suitable methods such as ELISA, immunofluorescence (IFA), focus reduction neutralization tests (FRNT), immunoprecipitation, or Western blotting.

According to a particular aspect of the use of the active ingredient, the combination or the composition of compounds of the invention, the active ingredients are designed to enable performing a prime-boost administration in a host in need thereof, where the first administration step elicits an immune, especially cellular, immune response and the later administration step(s) boost(s) the immune reaction including the cellular immune response. For each step of administration, it is preferred that the pseudotyping envelope protein(s) of the vector particles is (are) different from the one used in the other step(s), especially originate from different viruses, in particular different VSVs. In the prime-boost regimen, the administered combination of compounds of each step comprises lentiviral vectors as defined herein which collectively express all the antigenic polypeptides. Accordingly, active ingredients, combinations or compositions of compounds may be provided to perform the prime-boost regimen which comprise compounds that are distinct lentiviral particles at least due to the difference in their pseudotyping envelope proteins.

Accordingly, when a prime-boost regimen is selected, active ingredients, combinations or compositions of compounds containing said lentiviral vectors can be provided in separate packages or can be presented in a common package for a separate use thereof.

Therefore, the notice included in the packages and comprising the directions for use, may indicate the sequence order for the administration of the active ingredients, combinations or compositions of compounds and the time slot for their administration, for priming and subsequently boosting an immune reaction in a host.

In accordance with the invention when the active ingredients, combination or compositions of compounds are used in a prime-boost regimen, a first active ingredient/combination or composition of compounds is provided which contains lentiviral vector particles pseudotyped with a first determined pseudotyping envelope G protein obtained from the VSV, strain New-Jersey, and a second active ingredient/combination or composition of compounds is provided which contains lentiviral viral vector particles pseudotyped with a second determined pseudotyping envelope G protein obtained from a VSV, strain Indiana. The order of use in the prime-boost regimen of the first and second compounds thus described may alternatively be inversed. Thus, the lentiviral vector particles contained in the separate active ingredients/compounds of the combinations or compositions of the invention when intended for use in a prime-boost regiment are distinct from each other, at least due to the particular pseudotyping envelope protein(s) used for pseudotyping the vector particles.

In the examples which follow where mice models have been treated according to the prime-boost regimen with lentiviral vector particles of the invention, It has been shown by the inventors that mice immunized according to such a regimen and challenged after the last immunization step exhibit a sterile protection for a significant proportion of the vaccinated mice (more than 80%) which illustrates that the compounds of the invention elicit an effective protection in a host, and would therefore constitute a suitable candidate vaccine for immunization especially in a human host.

The invention relates, in a particular embodiment, to the active ingredients/combination or composition of compounds of the invention especially as lentiviral vector particles as defined herein, for the protective immunization against malaria parasite infection or against parasite-induced pathology in a mammalian host, especially in a human host, in a dosage regimen comprising separately provided active ingredients wherein the dose of the active ingredients intended for priming and boosting the cellular immune response is a moderate dose and the dose intended for boosting the cellular immune response is higher than the dose for priming.

Accordingly, the dose of lentiviral vectors intended for priming and boosting the cellular immune response which is used in the administration pattern, comprises from 10⁵ TU to 10¹⁰ TU of each type of viral particles especially from 10⁵ to 10⁷, when integrative vectors are used. The dose intended for priming and boosting comprises from 10⁷ to 10¹⁰ of each type of lentiviral particles when integrative-incompetent vectors are used. The invention also concerns the use of the combination of compounds of the invention especially as lentiviral vector according to the definitions given herein, for the manufacture of an immunogenic active ingredients or composition for prophylactic immunisation against malaria parasite infection or against parasite-induced pathology in a mammalian host, especially in a human host.

The invention also concerns a method of providing immunization in a mammalian host, especially in a human host, comprising the step of administering the active ingredients or the combination or composition of compounds of the invention especially as lentiviral vectors of the invention to elicit the immune response, and optionally repeating the administration steps one or several times, to boost said response, in accordance with the present disclosure.

In a particular embodiment of the invention, the active ingredient, the combination or the composition of compounds especially provided as lentiviral vector(s) may be used in association with an adjuvant compound suitable for administration to a mammalian, especially a human host, and/or with an immunostimulant compound, together with an appropriate delivery vehicle.

The active ingredients, combination or composition of compounds quoted above can be injected in a host via different routes: subcutaneous (s.c.), intradermal (i.d.), intramuscular (i.m.) or intravenous (i.v.) injection, oral administration and mucosal or skin administration, especially intranasal administration or inhalation. The quantity to be administered (dosage) depends on the subject to be treated, including considering the condition of the patient, the state of the individual's immune system, the route of administration and the size of the host. Suitable dosages range expressed with respect to the content in equivalent transducing units of vector particles (for HIV-1 lentiviral vectors) can be determined.

Other examples and features of the invention will be apparent when reading the examples and the figures which illustrate the preparation and application of the lentiviral vector particles with features that may be individually combined with the definitions given in the present description.

### DETAILED DESCRIPTION OF THE LENTIVIRAL VECTORS FOR USE ACCORDING TO THE INVENTION

The invention accordingly involves lentiviral vector which are lentiviral particles (i.e. vector particles), and which may be replication-incompetent lentiviral vectors, especially replication-incompetent HIV-1 based vectors characterized in that (i) they are pseudotyped with a determined heterologous viral envelope protein or viral envelope proteins originating from a RNA virus which is not HIV and (ii) they comprise in their genome at least one recombinant polynucleotide encoding at least one antigenic polypeptide (or polypeptide derivative thereof) carrying epitope(s) of a pre-erythrocytic stage antigen of a Plasmodium parasite or a polypeptidic derivative thereof wherein the parasite is capable of infecting a mammalian host, and wherein said epitope(s) encompass(es) T-epitope(s).

In a particular embodiment of the invention, the encoded chimeric antigenic polypeptide of pre-erythrocytic stage antigens of a Plasmodium parasite further comprises B-epitope(s).

The chimeric antigenic polypeptides or derivatives thereof expressed by the vectors are those disclosed herein in any aspects of the invention, in particular in the description of the combination of compounds of the invention.

According to a particular embodiment of the invention, the lentiviral vectors are either designed to express proficient (i.e., integrative-competent) or deficient (i.e., integrative-incompetent) particles.

The preparation of the lentiviral vectors is well known from the skilled person and has been extensively disclosed in the literature (confer for review Sakuma T. et al (Biochem. J. (2012) 443, 603-618). The preparation of such vectors is also illustrated herein in the Examples.

In a particular embodiment of the invention, the polynucleotide(s) encoding the antigenic polypeptides (ORF) of the lentiviral vector has (have) been mammal-codon optimized (CO) in particular human-codon optimized. Optionally the lentiviral sequences of the genome of said particles have also a mammal-codon optimized nucleotide sequence. In a particular aspect of the invention the codon optimization has been carried out for expression in mouse cells. In another embodiment the sequence the polynucleotide(s) encoding the antigenic polypeptides of the lentiviral vector has (have) been human-codon optimized (CO).

It has been observed that codon optimized nucleotide sequences, especially when optimized for expression in mammalian and in particular in human cells, enable the production of higher yield of particles in such mammalian or human cells. Production cells are illustrated in the examples. Accordingly, when lentiviral vector particles of the invention are administered to a mammalian, especially to a human host, higher amounts of particles are produced in said host which favour the elicitation of a strong immune response.

The lentiviral vector (i.e., lentiviral vectors particles or lentiviral-based vector particles) defined in the present invention are pseudotyped lentiviral vectors consisting of vector particles bearing envelope protein or envelope proteins which originate from a virus different from the particular lentivirus (especially a virus different from HIV, in particular HIV-1), which provides the vector genome of the lentiviral vector particles. Accordingly, said envelope protein or envelope proteins, are “heterologous” viral envelope protein or viral envelope proteins with respect to the vector genome of the particles. In the following pages, reference will also be made to “envelope protein(s)” to encompass any type of envelope protein or envelope proteins suitable to perform the invention. When reference is made to “lentiviral” vectors (lentiviral-based vectors) in the application, it relates in particular, to HIV-based vectors and especially HIV-1-based vectors.

The lentiviral vectors suitable to perform the invention are so-called replacement vectors, meaning that the sequences of the original lentivirus encoding the lentiviral proteins are essentially deleted in the genome of the vector or, when present, are modified, and especially mutated, especially truncated, to prevent expression of biologically active lentiviral proteins, in particular, in the case of HIV, to prevent the expression by said transfer vector, of functional ENV, GAG, and POL proteins and optionally of further structural and/or accessory and/or regulatory proteins of the lentivirus, especially of HIV. In a particular embodiment, the lentiviral vector is a first-generation vector, in particular a first-generation of a HIV-based vector which is characterized in that it is obtained using separate plasmids to provide (i) the packaging construct, (ii) the envelope and (iii) the transfer vector genome. Alternatively it may be a second-generation vector, in particular a second-generation of a HIV-based vector which in addition, is devoid of viral accessory proteins (such as in the case of HIV-1, Vif, Vpu, Vpr or Nef) and therefore includes only four out of nine HIV full genes: gag, pol, tat and rev. In another embodiment, the vector is a third-generation vector, in particular a third-generation of a HIV-based vector which is furthermore devoid of said viral accessory proteins and also is Tat-independent; these third-generation vectors may be obtained using 4 plasmids to provide the functional elements of the vector, including one plasmid encoding the Rev protein of HIV when the vector is based on HIV-1. Such vector system comprises only three of the nine genes of HIV-1. The structure and design of such generations of HIV-based vectors is well known in the art.

The “vector genome” of the vector particles is a recombinant nucleic acid which also comprises as a recombined sequence the polynucleotide or transgene of interest encoding one or more antigenic polypeptide(s) or polypeptide derivative thereof of malaria parasite as disclosed herein. The lentiviral-based sequence and polynucleotide/transgene of the vector genome are borne by a plasmid vector thus giving rise to the “transfer vector” also referred to as “sequence vector”. Accordingly, these expressions are used interchangeably in the present description. According to a particular embodiment, a vector genome prepared for the invention comprises a nucleic acid having a sequence selected in the group of SEQ ID No. 10, 13, 16, 19, 22, 25, 28, 31, 34, 37, 40, 43, 46, 48, 51, 54, 57, 60, 63, 66, 69, 72, 75, 78, 81 95, 97, 99, 101, 103, 105, 107, 109, 111, 113, 115, 117, 119, 121 or 123 or comprises a plurality of these sequences encoding antigenic polypeptides or derivatives thereof. The vector genome as defined herein accordingly contains, apart from the so-called recombinant polynucleotide(s) encoding the antigenic polypeptide(s) or polypeptide derivative thereof of malaria parasite placed under control of proper regulatory sequences for its expression, the sequences of the original lentiviral genome which are non-coding regions of said genome, and are necessary to provide recognition signals for DNA or RNA synthesis and processing (mini-viral genome). These sequences are cis-acting sequences necessary for packaging (w), reverse transcription (LTRs possibly mutated with respect to the original ones) and transcription and optionally integration (RRE) and furthermore for the particular purpose of the invention, they contain a functional sequence favouring nuclear import in cells and accordingly transgene transfer efficiency in said cells, which element is described as a DNA Flap element that contains or consists of the so-called central cPPT-CTS nucleotide domain present in lentiviral genome sequences especially in HIV-1 or in some retroelements such as those of yeasts.

The structure and composition of the vector genome used to prepare the lentiviral vectors of the invention are based on the principles described in the art and on examples of such lentiviral vectors primarily disclosed in (Zennou et al, 2000; Firat H. et al, 2002; VandenDriessche T. et al). Constructs of this type have been deposited at the CNCM (Institut Pasteur, France) as will be referred to herein. In this respect reference is also made to the disclosure, including to the deposited biological material, in patent applications WO 99/55892, WO 01/27300 and WO 01/27304.

According to a particular embodiment of the invention, a vector genome may be a replacement vector in which all the viral protein coding sequences between the 2 long terminal repeats (LTRs) have been replaced by the recombinant polynucleotide encoding the polypeptide of the malaria parasite, and wherein the DNA-Flap element has been re-inserted in association with the required cis-acting sequences described herein. Further features relating to the composition of the vector genome are disclosed in relation to the preparation of the particles.

In a particular embodiment of the invention one lentiviral vector encodes one antigenic polypeptide of the Plasmodium parasite.

In a particular embodiment, a lentiviral vector of the invention may comprise in its genome one or more than one recombinant polynucleotide encoding at least one antigenic polypeptide carrying epitope(s) of a pre-erythrocytic stage antigen as disclosed herein. In particular, said vector genome comprises two polynucleotides which are consecutive or separated on the genome and which encode different polypeptides of either the same or distinct antigens of the pre-erythrocytic stage of a Plasmodium parasite or different antigenic polypeptidic derivatives of distinct antigens of the parasite.

In a particular embodiment, the vector genome contains two or more recombinant polynucleotides, each of them encoding a distinct antigenic polypeptide and each polypeptide originating from a different antigen of the pre-erythrocytic stage as disclosed herein, including the CSP antigen and at least one of the Ag40 or Ag45 antigens, and optionally one or more, including all the antigenic polypeptides selected from the group of the thrombospondin related anonymous protein (TRAP), the inhibitor of cysteine protease (ICP), the metallopeptidase (Falcilysin), the GPI-anchored protein P113, the pore-forming like protein SPECT2 of the Plasmodium parasites disclosed herein or derivatives thereof.

The description made herein in respect to antigenic polypeptides similarly applies to polypeptidic derivatives thereof.

Particular features of the lentiviral vectors used in accordance with the various embodiments of the invention are also disclosed in the Examples, such features being either taken alone or in combination to produce the vectors.

According to the invention, the lentiviral vector particles are pseudotyped with a heterologous viral envelope protein or viral polyprotein of envelope originating from a RNA virus which is not the lentivirus providing the lentiviral sequences of the genome of the lentiviral particles.

As examples of typing envelope proteins for the preparation of the lentiviral vector, the invention relates to viral transmembrane glycosylated (so-called G proteins) envelope protein(s) of a Vesicular Stomatitis Virus (VSV), which is (are) for example chosen in the group of VSV-G protein(s) of the Indiana strain and VSV-G protein(s) of the New Jersey strain.

Other examples of VSV-G proteins that may be used to pseudotype the lentiviral vectors of the invention encompass VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURV), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV).

The envelope glycoprotein of the vesicular stomatitis virus (VSV-G) is a transmembrane protein that functions as the surface coat of the wild type viral particles. It is also a suitable coat protein for engineered lentiviral vectors. Presently, nine virus species are definitively classified in the VSV gender, and nineteen rhabdoviruses are provisionally classified in this gender, all showing various degrees of cross-neutralisation. When sequenced, the protein G genes indicate sequence similarities. The VSV-G protein presents a N-terminal ectodomain, a transmembrane region and a C-terminal cytoplasmic tail. It is exported to the cell surface via the transGolgi network (endoplasmic reticulum and Golgi apparatus).

Vesicular stomatitis Indiana virus (VSIV) and Vesicular stomatitis New Jersey virus (VSNJV) are preferred strains to pseudotype the lentiviral vectors of the invention, or to design recombinant envelope protein(s) to pseudotype the lentiviral vectors. Their VSV-G proteins are disclosed in GenBank, where several strains are presented. For VSV-G New Jersey strain reference is especially made to the sequence having accession number V01214. For VSV-G of the Indiana strain, reference is made to the sequence having accession number AAA48370.1 in Genbank corresponding to strain JO2428.

Said viral envelope protein(s) are capable of uptake by antigen presenting cells and especially by dendritic cells including by liver dendritic cells by mean of fusion and/or of endocytosis. In a particular embodiment, the efficiency of the uptake may be used as a feature to choose the envelope of a VSV for pseudotyping. In this respect the relative titer of transduction (Titer DC/Titer of other transduced cells e.g. 293T cells) may be considered as a test and envelope having a relative good ability to fuse with DC would be preferred.

Antigen Presenting Cells (APC) and especially Dendritic cells (DC) are proper target cells for pseudotyped lentiviral vectors which are used as immune compositions accordingly.

The VSV-G envelope protein(s) are expressed from a polynucleotide containing the coding sequence for said protein(s), which polynucleotide is inserted in a plasmid (designated envelope expression plasmid or pseudotyping env plasmid) used for the preparation of the lentiviral vector particles of the invention. The polynucleotide encoding the envelope protein(s) is under the control of regulatory sequences for the transcription and/or expression of the coding sequence (including optionally post-transcriptional regulatory elements (PRE) especially a polynucleotide such as the element of the Woodchuck hepatitis virus, i.e. the WPRE sequence, obtainable from Invitrogen).

Accordingly, a nucleic acid construct is provided which comprises an internal promoter suitable for the use in mammalian cells, especially in human cells in vivo and the nucleic acid encoding the envelope protein under the control of said promoter. A plasmid containing this construct is used for transfection or for transduction of cells suitable for the preparation of vector particles. Promoters may in particular be selected for their properties as constitutive promoters, tissue-specific promoters, or inducible promoters. Examples of suitable promoters encompass the promoters of the following genes: MHC Class1 promoters, human beta-2 microglobulin gene (β2M promoter), EF1α, human PGK, PPI (preproinsulin), thiodextrin, HLA DR invariant chain (P33), HLA DR alpha chain, Ferritin L chain or Ferritin H chain, Chymosin beta 4, Chymosin beta 10, Cystatin Ribosomal Protein L41, CMVie or chimeric promoters such as GAG (CMV early enhancer/chicken β actin) disclosed in Jones S. et al (Jones S. et al Human Gene Therapy, 20:630-640 (June 2009)).

These promoters may also be used in regulatory expression sequences involved in the expression of gag-pol derived proteins from the encapsidation plasmids, and/or to express the antigenic polypeptides from the transfer vector.

Alternatively, when the envelope expression plasmid is intended for expression in stable packaging cell lines, especially for stable expression as continuously expressed viral particles, the internal promoter to express the envelope protein(s) is advantageously an inducible promoter such as one disclosed in Cockrell A. S. et al. (Mol. Biotechnol. (2007) 36:184-204). As examples of such promoters, reference is made to tetracycline and ecdysone inducible promoters. The packaging cell line may be the STAR packaging cell line (ref Cockrell A. S. et al (2007), Ikedia Y. et al (2003) Nature Biotechnol. 21:569-572) or a SODk packaging cell line, such as SODk0 derived cell lines, including SODk1 and SODk3 (ref Cockrell A. S. et al (2007), Cockrell A; S. et al (2006) Molecular Therapy, 14:276-284, Xu K. et al. (2001), Kafri T. et al (1999) Journal of Virol. 73:576-584).

According to the invention, the lentiviral vector are the product recovered from co-transfection of mammalian cells, with:
a vector plasmid comprising (i) lentiviral, especially HIV-1, cis-active sequences necessary for packaging, reverse transcription, and transcription and further comprising a functional lentiviral, especially derived from HIV-1, DNA flap element and (ii) a polynucleotide encoding one or more chimeric antigenic polypeptide(s) (or polypeptide derivative thereof) of a malaria parasite as disclosed herein under the control of regulatory expression sequences, and optionally comprising sequences for integration into the genome of the host cell;
an expression plasmid encoding a pseudotyping envelope derived from a RNA virus, said expression plasmid comprising a polynucleotide encoding an envelope protein or proteins for pseudotyping, wherein said envelope pseudotyping protein is advantageously from a VSV and is in particular a VSV-G of the Indianan strain or of the New Jersey strain and,
an encapsidation plasmid, which either comprises lentiviral, especially HIV-1, gag-pol packaging sequences suitable for the production of integration-competent vector particles or modified gag-pol packaging sequences suitable for the production of integration-deficient vector particles.

The invention thus also concerns lentiviral vector particles as described above, which are the product recovered from a stable cell line transfected with:
a vector plasmid comprising (i) lentiviral, especially HIV-1, cis-active sequences necessary for packaging, reverse transcription, and transcription and further comprising a functional lentiviral, especially HIV-1, DNA flap element and optionally comprising cis-active sequences necessary for integration, said vector plasmid further comprising (ii) a polynucleotide of a codon-optimized sequence for murine or for human of the gene encoding the chimeric antigenic polypeptide (or a derivative thereof) of a Plasmodium parasite as disclosed herein, under the control of regulatory expression sequences, especially a promoter;
a VSV-G envelope expression plasmid comprising a polynucleotide encoding a VSV-G envelope protein in particular VSV-G of the Indiana strain or of the New Jersey strain, wherein said polynucleotide is under the control of regulating expression sequences, in particular regulatory expression sequences comprising an inducible promoter, and;
an encapsidation plasmid, wherein the encapsidation plasmid either comprises lentiviral, especially HIV-1, gag-pol coding sequences suitable for the production of integration-competent vector particles or modified gag-pol coding sequences suitable for the production of integration-deficient vector particles, wherein said gag-pol sequences are from the same lentivirus sub-family as the DNA flap element, wherein said lentiviral gag-pol or modified gag-pol sequence is under the control of regulating expression sequences.

The stable cell lines expressing the vector particles of the invention are in particular obtained by transduction of the plasmids.

The polynucleotide encodes at least one antigenic polypeptide of a malaria parasite according to any embodiment disclosed in the present specification. In particular, it encodes a polypeptide which is a truncated mammalian, especially human, codon-optimized sequence coding for such antigenic polypeptide of Plasmodium falciparum, Plasmodium vivax or Plasmodium berghei.

In a particular embodiment, the polynucleotide encodes antigenic polypeptides of the malaria parasite in particular chimeric antigenic polypeptides as defined herein. Accordingly, the vector plasmid may comprise one or several expression cassettes for the expression of the various antigenic polypeptides or may comprise bicistronic or multicistronic expression cassettes where the polynucleotides encoding the chimeric polypeptide and optionally additional various polypeptides are separated by an IRES sequence of viral origin (Internal Ribosome Entry Site), or it may encode fusion protein(s).

The internal promoter contained the vector genome and controlling the expression of the polynucleotide encoding a chimeric antigenic polypeptide of the malaria parasite (as a transgene or in an expression cassette) and optionally additional antigens or derivatives thereof may be selected from the promoters of the following genes: MHC Class 1 promoters, such as human beta-2 microglobuline gene (32M promoter), or EF1α, human PGK, PPI (preproinsulin), thiodextrin, HLA DR invariant chain (P33), HLA DR alpha chain, Ferritin L chain or Ferritin H chain, Chymosin beta 4, Chimosin beta 10, or Cystatin Ribosomal Protein L41 CMVie or chimeric promoters such as GAG (CMV early enhancer/chicken β actin) disclosed in Jones S. et al (2009).

A promoter among the above cited internal promoters may also be selected for the expression of the envelope protein(s) and packaging (gag-pol derived) proteins. Alternatively, vector particles can be produced from co-transfection of the plasmids disclosed herein, in stable packaging cell lines which thus become capable of continuously secreting vector particles. Promoters used in the regulatory expression sequences involved for the expression of the envelope protein(s) are advantageously inducible promoters.

The following particular embodiments may be carried out when preparing the lentiviral vector based on human lentivirus, and especially based on HIV-1 virus.

According to the invention, the genome of the lentiviral vector is derived from a human lentivirus, especially from the HIV lentivirus. In particular, the pseudotyped lentiviral vector is an HIV-based vector, such as an HIV-1, or HIV-2 based vector, in particular is derived from HIV-1M, for example from the BRU or LAI isolates. Alternatively, the lentiviral vector providing the necessary sequences for the vector genome may be originating from lentiviruses such as EIAV, CAEV, VISNA, FIV, BIV, SIV, HIV-2, HIV—O which are capable of transducing mammalian cells.

As stated above, when considering it apart from the recombinant polynucleotide that it finally contains, the vector genome is a replacement vector in which the nucleic acid between the 2 long terminal repeats (LTRs) in the original lentivirus genome have been restricted to cis-acting sequences for DNA or RNA synthesis and processing, including for the efficient delivery of the transgene to the nuclear of cells in the host, or at least are deleted or mutated for essential nucleic acid segments that would enable the expression of lentiviral structure proteins including biological functional GAG polyprotein and possibly POL and ENV proteins.

In a particular embodiment, the 5′ LTR and 3′ LTR sequences of the lentivirus are used in the vector genome, but the 3′-LTR at least is modified with respect to the 3′LTR of the original lentivirus at least in the U3 region which for example can be deleted or partially deleted for the enhancer. The 5′LTR may also be modified, especially in its promoter region where for example a Tat-independent promoter may be substituted for the U3 endogenous promoter.

In a particular embodiment the vector genome comprises one or several of the coding sequences for Vif-, Vpr, Vpu- and Nef-accessory genes (for HIV-1 lentiviral vectors). Alternatively, these sequences can be deleted independently or each other or can be non-functional (second-generation lentiviral vector).

The vector genome of the lentiviral vector particles comprises, as an inserted cis-acting fragment, at least one polynucleotide consisting in the DNA flap element or containing such DNA flap element. In a particular embodiment, the DNA flap is inserted upstream of the polynucleotide encoding the chimeric antigenic polypeptide of Plasmodium parasite, and similarly upstream of the polynucleotide encoding additional antigenic polypeptide of Plasmodium parasite if any and is advantageously—although not necessarily—located in an approximate central position in the vector genome. A DNA flap suitable for the invention may be obtained from a retrovirus, especially from a lentivirus, in particular a human lentivirus especially a HIV-1 retrovirus, or from a retrovirus-like organism such as retrotransposon. It may be alternatively obtained from the CAEV (Caprine Arthritis Encephalitis Virus) virus, the EIAV (Equine Infectious Anaemia Virus) virus, the VISNA virus, the SIV (Simian Immunodeficiency Virus) virus or the FIV (Feline Immunodeficiency Virus) virus. The DNA flap may be either prepared synthetically (chemical synthesis) or by amplification of the DNA providing the DNA Flap from the appropriate source as defined above such as by Polymerase chain reaction (PCR). In a more preferred embodiment, the DNA flap is obtained from an HIV retrovirus, for example HIV-1 or HIV-2 virus including any isolate of these two types.

The DNA flap (also designated cPPT/CTS) (defined in Zennou V. et al. ref 27, 2000, Cell vol 101, 173-185 or in WO 99/55892 and WO 01/27304), is a structure which is central in the genome of some lentiviruses especially in HIV, where it gives rise to a 3-stranded DNA structure normally synthesized during especially HIV reverse transcription and which acts as a cis-determinant of HIV genome nuclear import. The DNA flap enables a central strand displacement event controlled in cis by the central polypurine tract (cPPT) and the central termination sequence (CTS) during reverse transcription. When inserted in lentiviral-derived vectors, the polynucleotide enabling the DNA flap to be produced during reverse-transcription, stimulates gene transfer efficiency and complements the level of nuclear import to wild-type levels (Zennou et al., Cell, 2000 Cell vol 101, 173-185 or in WO 99/55892 and WO 01/27304).

Sequences of DNA flaps have been disclosed in the prior art, especially in the above cited patent applications. These sequences are also disclosed in the sequence of SEQ ID No 1 from position 2056 to position 2179. They are preferably inserted as a fragment, optionally with additional flanking sequences, in the vector genome, in a position which is preferably near the centre of said vector genome. Alternatively they may be inserted immediately upstream from the promoter controlling the expression of the polynucleotide(s) encoding the antigenic polypeptide. Said fragments comprising the DNA flap, inserted in the vector genome may have a sequence of about 80 to about 200 bp, depending on its origin and preparation.

According to a particular embodiment, a DNA flap has a nucleotide sequence of about 90 to about 140 nucleotides.

In HIV-1, the DNA flap is a stable 99-nucleotide-long plus strand overlap. When used in the genome vector of the lentiviral vector of the invention, it may be inserted as a longer sequence, especially when it is prepared as a PCR fragment. A particular appropriate polynucleotide comprising the structure providing the DNA flap is a 124-base pair polymerase chain reaction (PCR) fragment encompassing the cPPT and CTS regions of the HIV-1 DNA (as disclosed in SEQ ID N No. 1).

It is specified that the DNA flap used in the genome vector and the polynucleotides of the encapsidation plasmid encoding the GAG and POL polyproteins should originate from the same lentivirus sub-family or from the same retrovirus-like organism.

Preferably, the other cis-activating sequences of the genome vector also originate from the same lentivirus or retrovirus-like organism, as the one providing the DNA flap. The vector genome may further comprise one or several unique restriction site(s) for cloning the recombinant polynucleotide.

In a preferred embodiment, in said vector genome, the 3′ LTR sequence of the lentiviral vector genome is devoid of at least the activator (enhancer) and possibly the promoter of the U3 region. In another particular embodiment, the 3′ LTR region is devoid of the U3 region (delta U3). In this respect, reference is made to the description in WO 01/27300 and WO 01/27304.

In a particular embodiment, in the vector genome, the U3 region of the LTR 5′ is replaced by a non lentiviral U3 or by a promoter suitable to drive tat-independent primary transcription. In such a case, the vector is independent of tat transactivator (third generation vector).

The vector genome also comprises the psi (Ψ) packaging signal. The packaging signal is derived from the N-terminal fragment of the gag ORF. In a particular embodiment, its sequence could be modified by frameshift mutation(s) in order to prevent any interference of a possible transcription/translation of gag peptide, with that of the transgene.

The vector genome may optionally also comprise elements selected among a splice donor site (SD), a splice acceptor site (SA) and/or a Rev-responsive element (RRE). According to a particular embodiment, the vector plasmid (or added genome vector) comprises the following cis-acting sequences for a transgenic expression cassette:
1. The LTR sequence (Long-Terminal Repeat), required for reverse transcription, the sequences required for transcription and including optionally sequences for viral DNA integration. The 3′ LTR is deleted in the U3 region at least for the promoter to provide SIN vectors (Self-inactivating), without perturbing the functions necessary for gene transfer, for two major reasons: first, to avoid trans-activation of a host gene, once the DNA is integrated in the genome and secondly to allow self-inactivation of the viral cis-sequences after retro transcription. Optionally, the tat-dependent U3 sequence from the 5′-LTR which drives transcription of the genome is replaced by a non endogenous promoter sequence. Thus, in target cells only sequences from the internal promoter will be transcribed (transgene).
2. The Ψ region, necessary for viral RNA encapsidation.
3. The RRE sequence (REV Responsive Element) allowing export of viral messenger RNA from the nucleus to the cytosol after binding of the Rev protein.
4. The DNA flap element (cPPT/CTS) to facilitate nuclear import.
5. Optionally post-transcriptional regulatory elements, especially elements that improve the expression of the antigenic polypeptides in dendritic cells, such as the WPRE cis-active sequence (Woodchuck hepatitis B virus Post-Responsive Element) also added to optimize stability of mRNA (Zufferey et al., 1999), the matrix or scaffold attachment regions (SAR and MAR sequences) such as those of the immunoglobulin-kappa gene (Park F. et al Mol Ther 2001; 4:164-173).

The lentiviral vector of the invention is non replicative (replication-incompetent) i.e., the vector and lentiviral vector genome are regarded as suitable to alleviate concerns regarding replication competent lentiviruses and especially are not able to form new particles budding from the infected host cell after administration. This may be achieved in well known ways as the result of the absence in the lentiviral genome of the gag, pol or env genes, or their absence as “functional genes”. The gag and pol genes are thus, only provided in trans. This can also be achieved by deleting other viral coding sequence(s) and/or cis-acting genetic elements needed for particles formation. By “functional” it is meant a gene that is correctly transcribed, and/or correctly expressed. Thus, if present in the lentiviral vector genome of the invention in this embodiment contains sequences of the gag, pol, or env are individually either not transcribed or incompletely transcribed; the expression “incompletely transcribed” refers to the alteration in the transcripts gag, gag-pro or gag-pro-pol, one of these or several of these being not transcribed. Other sequences involved in lentiviral replication may also be mutated in the vector genome, in order to achieve this status. The absence of replication of the lentiviral vector should be distinguished from the replication of the lentiviral genome. Indeed, as described before, the lentiviral genome may contain an origin of replication ensuring the replication of the lentiviral vector genome without ensuring necessarily the replication of the vector particles.

In order to obtain lentiviral vectors according to the invention, the vector genome (as a vector plasmid) must be encapsidated in particles or pseudo-particles. Accordingly, lentiviral proteins, except the envelope proteins, have to be provided in trans to the vector genome in the producing system, especially in producing cells, together with the vector genome, having recourse to at least one encapsidation plasmid carrying the gag gene and either the pol lentiviral gene or an integrative-incompetent pol gene, and preferably lacking some or all of the coding sequences for Vif-, Vpr, Vpu- and Nef-accessory genes and optionally lacking Tat (for HIV-1 lentiviral vectors).

A further plasmid is used, which carries a polynucleotide encoding the envelope pseudotyping protein(s) selected for pseudotyping lentiviral vector particles. In a preferred embodiment, the packaging plasmid encodes only the lentiviral proteins essential for viral particle synthesis. Accessory genes whose presence in the plasmid could raise safety concerns are accordingly removed. Accordingly, viral proteins brought in trans for packaging are respectively as illustrated for those originating from HIV-1:
1. GAG proteins for building of the matrix (MA, with apparent Molecular Weight p17), the capsid (CA, p24) and nucleocapsid (NC, p6).
2. POL encoded enzymes: integrase, protease and reverse transcriptase.
3. TAT and REV regulatory proteins, when TAT is necessary for the initiation of LTR-mediated transcription; TAT expression may be omitted if the U3 region of 5′LTR is substituted for a promoter driving tat-independent transcription. REV may be modified and accordingly used for example in a recombinant protein which would enable recognition of a domain replacing the RRE sequence in the vector genome, or used as a fragment enabling binding to the RRE sequence through its RBD (RNA Binding Domain).

In order to avoid any packaging of the mRNA generated from the genes contained in the packaging plasmid in the viral particles, the w region is removed from the packaging plasmid. A heterologous promoter is inserted in the plasmid to avoid recombination issues and a poly-A tail is added 3′ from the sequences encoding the proteins. Appropriate promoters have been disclosed above.

The envelope plasmid encodes the envelope protein(s) for pseudotyping which are disclosed herein, under the control of an internal promoter, as disclosed herein.

Any or all the described plasmids for the preparation of the lentiviral vector particles of the invention may be codon optimized (CO) in the segment encoding proteins. Codon optimization according to the invention is preferably performed to improve translation of the coding sequences contained in the plasmids, in mammalian cells, murine or especially human cells. According to the invention, codon optimization is especially suited to directly or indirectly improve the preparation of the vector particles or to improve their uptake by the cells of the host to whom they are administered, or to improve the efficiency of the transfer of the polynucleotide encoding the antigenic polypeptide of the malaria parasite (transgene) in the genome of the transduced cells of the host. Methods for optimizing codons are well known in the art and codon optimization is especially performed using available programs to that effect. Codon optimization is illustrated for the coding sequences used in the examples. In a particular embodiment of the invention, the pseudotyped lentiviral vector is also, or alternatively, integrative-competent, thus enabling the integration of the vector genome and of the recombinant polynucleotide which it contains into the genome of the transduced cells or in the cells of the host to whom it has been administered. In another particular embodiment of the invention, the pseudotyped lentiviral vector is also, or alternatively, integrative-incompetent. In such a case, the vector genome and thus the recombinant polynucleotide which it contains do not integrate into the genome of the transduced cells or in the cells of the host to whom it has been administered. The present invention relates to the use of a lentiviral vector wherein the expressed integrase protein is defective and which further comprises a polynucleotide especially encoding at least one antigenic polypeptide carrying epitope(s) of a pre-erythrocytic stage antigen of a Plasmodium parasite, in an immunogenic composition.

By “integration-incompetent”, it is meant that the integrase, preferably of lentiviral origin, is devoid of the capacity of integration of the lentiviral genome into the genome of the host cells i.e., an integrase protein mutated to specifically alter its integrase activity.

Integration-incompetent lentiviral vectors are obtained by modifying the polgene encoding the Integrase, resulting in a mutated pol gene encoding an integrative deficient integrase, said modified pol gene being contained in the encapsidation plasmid. Such integration-incompetent lentiviral vectors have been described in patent application WO 2006/010834. Accordingly the integrase capacity of the protein is altered whereas the correct expression from the encapsidation plasmid of the GAG, PRO and POL proteins and/or the formation of the capsid and hence of the vector particles, as well as other steps of the viral cycle, preceding or subsequent to the integration step, such as the reverse transcription, the nuclear import, stay intact. An integrase is said defective when the integration that it should enable is altered in a way that an integration step takes place less than 1 over 1000, preferably less than 1 over 10000, when compared to a lentiviral vector containing a corresponding wild-type integrase.

In a particular embodiment of the invention, the defective integrase results from a mutation of class 1, preferably amino acid substitutions (one-amino acid substitution) or short deletions fulfilling the requirements of the expression of a defective integrase. The mutation is carried out within the pol gene. These vectors may carry a defective integrase with the mutation D64V in the catalytic domain of the enzyme, which specifically blocks the DNA cleaving and joining reactions of the integration step. The D64V mutation decreases integration of pseudotyped HIV-1 up to 1/10,000 of wild type, but keep their ability to transduce non dividing cells, allowing efficient transgene expression.

Other mutations in the pol gene which are suitable to affect the integrase capacity of the integrase of HIV-1 are the following: H12N, H12C, H16C, H16V, S81 R, D41A, K42A, H51A, Q53C, D55V, D64E, D64V, E69A, K71A, E85A, E87A, D116N, D1161, D116A, N120G, N120I, N120E, E152G, E152A, D-35-E, K156E, K156A, E157A, K159E, K159A, K160A, R166A, D167A, E170A, H171A, K173A, K186Q, K186T, K188T, E198A, R199C, R199T, R199A, D202A, K211A, Q214L, Q216L, Q221 L, W235F, W235E, K236S, K236A, K246A, G247W, D253A, R262A, R263A and K264H. In a particular embodiment, mutation in the pol gene is performed at either of the following positions D64, D116 or E152, or at several of these positions which are in the catalytic site of the protein. Any substitution at these positions is suitable, including those described above.

Another proposed substitution is the replacement of the amino acids residues RRK (positions 262 to 264) by the amino acids residues AAH.

In a particular embodiment of the invention, when the lentiviral vector is integration-incompetent, the lentiviral genome further comprises an origin of replication (ori), whose sequence is dependent on the nature of cells where the lentiviral genome has to be expressed. Said origin of replication may be from eukaryotic origin, preferably of mammalian origin, most preferably of human origin. It may alternatively be of viral origin, especially coming from DNA circular episomic viruses, such as SV40 or RPS. It is an advantageous embodiment of the invention to have an origin or replication inserted in the lentiviral genome of the lentiviral vector of the invention. Indeed, when the lentiviral genome does not integrate into the cell host genome (because of the defective integrase), the lentiviral genome is lost in cells that undergo frequent cell divisions; this is particularly the case in immune cells, such as B or T cells. The presence of an origin of replication ensures that at least one lentiviral genome is present in each cell, even after cell division, accordingly maximizing the efficiency of the immune response.

The lentiviral vector genome of said lentiviral vectors of the invention may especially be derived from HIV-1 plasmid pTRIPΔU3.CMV-GFP deposited at the CNCM (Paris, France) on Oct. 11, 1999 under number I-2330 (also described in WO01/27300) or variants thereof. The sequence of such variants are provided as SEQ ID No. 1 or 2. When the vector genome is derived from these particular plasmids, a sequence of a recombinant polynucleotide encoding an antigenic polypeptide of a Plasmodium parasite as disclosed in the present application is inserted therein, in addition or in replacement of the GFP coding fragment. The GFP coding sequence may also be substituted by a different marker. The CMV promoter may also be substituted by another promoter, especially one of the promoters disclosed above, especially in relation to the expression of the transgene.

The WPRE sequence also contained in the particular deposited pTRIP vectors may optionally be deleted.

Vector particles may be produced after transfection of appropriate cells (such as mammalian cells or human cells, such as Human Embryonic Kidney cells illustrated by 293 T cells) by said plasmids, or by other processes. In the cells used for the expression of the lentiviral particles, all or some of the plasmids may be used to stably express their coding polynucleotides, or to transiently or semi-stably express their coding polynucleotides.

The concentration of particles produced can be determined by measuring the P24 (capsid protein for HIV-1) content of cell supernatants.

The lentiviral vector of the invention, once administered into the host, infects cells of the host, possibly specific cells, depending on the envelope proteins it was pseudotyped with. The infection leads to the release of the lentiviral vector genome into the cytoplasm of the host cell where the retrotranscription takes place. Once under a triplex form (via the DNA flap), the lentiviral vector genome is imported into the nucleus, where the polynucleotide(s) encoding polypeptide(s) of antigen(s) of the malaria parasite is (are) expressed via the cellular machinery. When non-dividing cells are transduced (such as DC), the expression may be stable. When dividing cells are transduced, such as B cells, the expression is temporary in absence of origin of replication in the lentiviral genome, because of nucleic acid dilution and cell division. The expression may be longer by providing an origin of replication ensuring a proper diffusion of the lentiviral vector genome into daughter cells after cell division. The stability and/or expression may also be increased by insertion of MAR (Matrix Associated Region) or SAR (Scaffold Associated Region) elements in the vector genome.

Indeed, these SAR or MAR regions are AT-rich sequences and enable to anchor the lentiviral genome to the matrix of the cell chromosome, thus regulating the transcription of the polynucleotide encoding at least one antigenic polypeptide, and particularly stimulating gene expression of the transgene and improving chromatin accessibility.

If the lentiviral genome is non integrative, it does not integrate into the host cell genome. Nevertheless, the at least one polypeptide encoded by the transgene is sufficiently expressed and longer enough to be processed, associated with MHC molecules and finally directed towards the cell surface. Depending on the nature of the polynucleotide(s) encoding antigenic polypeptide(s) of a malaria parasite, the at least one polypeptide epitope associated with the MHC molecule triggers a humoral or a cellular immune response.

Unless otherwise stated, or unless technically not relevant, the characteristics disclosed in the present application with respect to any of the various features, embodiments or examples of the structure or use of the lentiviral particles, especially regarding their envelope protein(s), or the recombinant polynucleotide, may be combined according to any possible combinations.

The invention further relates to a combination of compounds for separate administration to a mammalian host, which comprises at least:
(i) lentiviral vector particles of the invention which are pseudotyped with a first determined heterologous viral envelope pseudotyping protein or viral envelope pseudotyping proteins; such first pseudotyping protein may be from the New-Jersey strain of VSV;
(ii) provided separately from lentiviral vector particles in (i), lentiviral vector particles of the invention which are pseudotyped with a second determined heterologous viral envelope pseudotyping protein or viral envelope pseudotyping proteins distinct from said first heterologous viral envelope pseudotyping protein(s); such second pseudotyping protein may be from the Indiana strain of VSV.

The invention also relates to a polynucleotide which is a codon-optimized nucleic acid encoding a pre-erythrocytic stage chimeric antigen of a Plasmodium parasite, wherein said polynucleotide is selected from the group of: SEQ ID No. 115, SEQ ID No. 117, SEQ ID No. 119, SEQ ID No. 121.

Codon optimisation reflected in the above sequences has been carried out for expression in mice when polynucleotides encoding antigens of P. berghei are concerned. It has been carried out for expression in human cells when polynucleotides encoding antigens of P. falciparum or of P. vivax are concerned.

The invention also concerns the use of the above polynucleotides for the design of alternative forms of nucleic acids also suitable for the preparation of the vectors of the invention, wherein the thus obtained nucleic acids are RNAs of modified DNAs such as threose nucleic acids (TNAs), glycol nucleic acids (GNAs), peptide nucleic acids (PNAs), locked nucleic acids (LNAs) with either known configuration or ethylene nucleic acids (ENA) or cyclohexenyl nucleic acids (CeNA) or hybrids or combinations thereof. In particular when carrying out the preparation of the vector genome of the invention, hybrid molecules can be used wherein the polynucleotide encoding the antigenic polypeptide of the malaria parasite as disclosed herein is expressed from one of the above disclosed forms of sequences. According to an embodiment of the invention, the nucleotide sequence of the vector genome is a chimeric sequence encompassing a modified nucleic acid for the transcription of the antigenic polypeptide. In another embodiment of the invention, possibly in combination with the above disclosed alternative forms of the nucleic acid, the polynucleotide encoding the antigenic polypeptide is structurally modified and/or chemically modified. Illustrative thereof a polynucleotide comprises a Kozak consensus sequence in its 5′ region. Such polynucleotides encompassing Kozak consensus sequences are especially illustrated herein. Other nucleic acid sequences that are not of lentiviral origin may be present in the vector genome are IRES sequence(s) (Internal Ribosome entry site) suitable to initiate polypeptide synthesis WPRE sequence as post-transcriptional regulatory element to stabilize the produced RNA.

According to another embodiment of the invention, if multiple heterologous polypeptides are encoded by one vector genome, the coding sequences may optionally be separated by a linker moiety which is either a nucleic acid based molecule or a non-nucleic acid based molecule. Such a molecule may be a functionalized linker molecule aimed at recognizing a 3′ functionalized nucleic acid to which it shall be linked. A sequence suitable to function as a linker may alternatively be a nucleic acid which encodes a self-cleaving peptide, such as a 2A peptide.

The invention relates to a collection of polynucleotides thus described wherein a polynucleotide encodes a chimeric antigenic polypeptide as defined herein and another polynucleotide encodes one or more additional antigenic polypeptides of the malaria parasite as described herein for the purpose of the invention, provided the collection of these polynucleotides is suitable for the preparation of the active ingredients of the combination or composition of compounds of the invention. The invention also relates to the use of the polynucleotides thus disclosed, for the preparation of a collection of lentiviral vectors, in particular HIV-1 based vectors, wherein a vector comprises in its genome, at least one of these polynucleotides, provided the collection of lentiviral vectors enables the expression of all antigenic polypeptides encoded by the polynucleotides.

Further features and properties of the present invention, including features to be used in the embodiments described above will be described in the examples and figures which follow and may accordingly be used to characterise the invention.

### LEGENDS OF THE FIGURES

FIG. 1. Schema of plasmids used in the production of Lentiviral Particles.

FIG. 2. C57BL/6 mice (n=5) were immunized intramuscularly with 5×10⁷ TU of VSV^{IND} pseudotyped lentiviral particles coding for the antigens, CSP, Celtos SPECT, HSP20 and Ag13. As a positive control of protection, mice were immunized with 50k irradiated sporozoites via intravenous injection. Thirty days after immunization, the animals were challenged with 10,000 bioluminescent sporozoites micro-injected subcutaneously in the mice footpad. The parasite load in the liver was quantified two days later by bioluminescence as shown in the picture for CSP, Celtos and Ag13. The graph shows the quantification of the liver infection represented as the log of average radiance (squares). Dotted line represents the average of background signal (Bk) of a non-infected region. *P<0.05 and ***P<0.001 (ANOVA).

FIG. 3. C57BL/6 mice (n=5 per group) were immunized or not (naïve) with 5×10⁷ TU of VSV^{IND} LPs carrying Ag13 (negative control) and CSP (positive control). The groups receiving concentrated LPs were inoculated intramuscularly in the thigh muscle with 50 uL of vector (Ag13 im c and CSP im c). The groups receiving non-concentrated LPs were inoculated intraperitoneally with 700 μL of vector (Ag13 ip nc and CSP ip nc). Thirty days after immunization, the animals were challenged with 5,000 luciferase-expressing sporozoites, micro-injected subcutaneously in the mice footpad. The parasite load in the liver was quantified two days later by bioluminescence as shown in the FIG. 2. The graph shows the average and sd of the log of average radiance in the liver two days after SPZ inoculation. Dotted line represents the average of background signal (Bk). ns, not significant (ANOVA).

FIG. 4. C57BL/6 mice (n=4-5 per group) were intraperitoneally immunized or not (naïve) with 1×10⁷ TU of non concentrated VSV^{IND} CSP LPs under the control of CMV or B2M promoters (CMV CSP and B2M CSP, respectively). Thirty days after immunization, the animals were challenged with 5,000 luciferase-expressing sporozoites micro-injected subcutaneously in the mice footpad. The parasite load in the liver was quantified two days later by bioluminescence as shown in the FIG. 2. The graph shows the average and sd of the log of average radiance in the liver two days after SPZ inoculation. Dotted line represents the average of background signal (Bk). *P<0.05; ns, not significant (ANOVA).

FIG. 5. 4 and 7 weeks-old C57BL/6 mice (n=4-per group) were acclimated for 3 weeks (old groups) and 3 days (new groups). These age-matched groups were then intraperitoneally immunized with 1×10⁷ TU of non concentrated VSV^{IND} B2M CSP or GFP LPs. Thirty days after immunization, the animals were challenged with 5,000 luciferase-expressing sporozoites micro-injected subcutaneously in the mice footpad. The parasite load in the liver was quantified two days later by bioluminescence as shown in the FIG. 2. The graph shows the average and sd of the log of average radiance in the liver two days after SPZ inoculation. Dotted line represents the average of background signal (Bk). *P<0.05; ns, not significant (ANOVA).

FIG. 6. 4 weeks-old C57BL/6 mice (n=4-per group) were acclimated for 3 weeks (old groups) and intraperitoneally immunized with different doses of non-concentrated VSV^{IND} B2M CSP (black) or GFP (white) LPs. Thirty days after immunization, the animals were challenged with 5,000 luciferase-expressing sporozoites micro-injected subcutaneously in the mice footpad. The parasite load in the liver was quantified two days later by bioluminescence as shown in the FIG. 2. The graph shows the average and sd of the log of average radiance in the liver two days after SPZ inoculation. Dotted line represents the average of background signal (Bk). *P<0.05; ***P<0.001; ns, not significant (ANOVA).

FIG. 7. Analysis of Sporozoite, Liver Stage and Blood Stage cDNA libraries of Plasmodium berghei (Pb) and falciparum (Pf) deposited in Plasmodb. The percentage of each expression sequence tag (EST) was normalized to the total number of ESTs and represented cumulatively. Each symbol represents one gene, ranked by EST abundance (higher to lower) and represented as % of total ESTs. Of note ˜10% of genes (most abundant) are responsible for ˜50% of total ESTs (dotted lines).

FIG. 8. Expression and surface localization of antigens. GFP-expressing Pb sporozoites were fixed with 2% of PFA and permeabilized with 0.1% of Triton X100 (perm) or not (live). Parasites were incubated with the indicated immune-sera (1/50) for one hour on ice, washed and revealed with goat anti-mouse secondary antibody labelled with AlexaFluor 647. Sporozoites were then analysed by cytometry as shown in the right histograms (surface, staining using live non-permeabilized SPZ; permeabilized, staining using fixed and permeabilized SPZ) or by fluorescence microscopy, as depicted in the pictures. Notice that CSP and antigen 9-6 present a surface pattern staining both by cytometry and microscopy.

FIG. 9. Targeted screening of protective antigens. 4 weeks-old C57BL/6 mice (n=5 per group) were acclimated for 3 weeks and intraperitoneally immunized with a single dose of 1×10⁷ TU of non-concentrated VSV^{IND} B2M LPs. Thirty days after immunization, the animals were challenged with 5,000 GFP-expressing sporozoites micro-injected subcutaneously in the mice footpad. The parasite infection was measured by flow cytometry. The graph shows the average of the log of parasitemia (trace, individual mice represented by circles) immunized with the indicated plasmodial antigens. Bold dotted lines represent the 95% tolerance interval of GFP log normal distribution. Mice with parasitemia below the lower limit of the tolerance interval are considered protected. Top dotted line is the average of control and bottom dotted line represents non-infected (NI) mice.

FIG. 10. Comparison of protection induced by one or two immunization doses. 4 weeks-old C57BL/6 mice (n=5 per group) were acclimated for 3 weeks and intraperitoneally immunized with a first dose of 5×10⁵ TU of non-concentrated VSV^{NJ} B2M LPs. Thirty days after the first immunization, the animals received a second dose of 1×10⁷ TU of non-concentrated VSV^{IND} B2M LPs. Thirty days later, mice were challenged with 5,000 GFP-expressing sporozoites micro-injected subcutaneously in the footpad. The parasite infection was measured by flow cytometry. The graph shows the log of parasitemia at day 5 post-inoculation of individual challenged mice that received two immunization doses (Squares, PB). Circles represent mice that received only one immunization dose of LPs (data from experiment shown in FIG. 9). Traces represents the average of the Log Parasitemia. Bold dotted lines represent the 95% tolerance interval of GFP log normal distribution. Mice below the lower limit of tolerance interval are considered protected. NI, non-infected mice.

FIG. 11. Targeted Screening of Protective Antigens. 4 weeks-old C57BL/6 mice (n=5-10 per group) were acclimated for 3 weeks and intraperitoneally immunized with a first dose of 5×10⁵ TU of non-concentrated VSV^{NJ} B2M LPs. Thirty days after the first immunization, the animals received a second dose of 1×10⁷ TU of non-concentrated VSV^{IND} B2M LPs. Third days later, mice were challenged with 5,000 GFP-expressing sporozoites micro-injected subcutaneously in the footpad. The parasite blood infection was measured by flow cytometry. (a.) The upper graph shows the log of parasitemia of individual mouse at day 5 post-infection. Traces represent the mean of the log parasitemia. The average of the GFP group (control of protection) is represented by the dotted middle line. The superior and inferior dotted lines delineate the 95% tolerance interval (grey box) of the GFP control group. The CSP group is the positive control of protection. NI (not infected=no parasitemia at day 10 post-infection, located at the limit of detection of our method of parasitemia quantification). Black circles represent antigens where there was a significant decrease in the averaged log parasitemia and therefore are considered protective (ANOVA). (b) The bottom graph represents the percentage of protected mice (% of animals below the 95% tolerance interval). Black bars represent protective antigens (Fisher's Exact test). *P<0.05, **P<0.01, ****P<0.0001.

FIG. 12. Structure of P. berghei protective antigens. Conserved structural and functional domains are represented by boxes according to the code on the right. GPI (glycosylphosphatidylinositol), TSR (thrombospondin type I repeat), MACPF (membrane attack complex/perforin).

FIG. 13. Protective antigens are conserved among plasmodial species. Amino acid sequences of protective orthologous antigens from rodent-infecting P. berghei,macaque-infecting P. cynomolgi, and human-infecting P. falciparum and P. vivax parasites were aligned by Multiple Sequence Comparison by Log-Expectation (MUSCLE). Vertical black bars represent identical amino acids conserved in the four plasmodial species, short dark gray bars represent repetitive regions and short light gray bars, insertional gaps used for the alignment.

FIG. 14. Protection induced by combination of down-selected protective antigens with a sub-optimal dose of CSP. Mice were immunized twice, four weeks apart, with a sub-optimal dose of CSP (5×10⁵ TU of non-concentrated VSV^{NJ} B2M LP in the first immunization and 5×10⁶ TU of non-concentrated VSV^{IND} B2M LP in the second immunization, white triangle, CSP) and the usual dose of protective plasmodial antigens (CSP+11-03, +11-05, +11-06, +11-07, +11-09 and +11-10; triangles). As negative control mice were immunized with the usual, two doses of GFP. 4 weeks after the second immunization dose, animals were challenged with 5,000 sporozoites.

FIG. 15. Sterile protection induced by a multigenic combination. Mice were immunized twice, four weeks apart, with 7× the individual dose (1 dose=5×10⁵ TU of non-concentrated VSV^{NJ} B2M LPs in the first immunization/1×10⁷ TU of non-concentrated VSV^{IND} B2M LPs in the second immunization) of the control antigen AL11-luciferase (Luc, white triangles), with the individual dose of CSP plus 6×Luc (gray triangles), or with the individual doses of CSP and of 6 conserved PE antigens (11-05, 11-06, 11-07, 11-09, 11-10 and 18-10; black triangles, 7cPEAg). 4 weeks after the second immunization dose, mice were challenged with 5,000 GFP SPZs. Both graphs show the individual log of parasitemia at day 5 post-challenge. (a) The graph shows the pooled results of three independent experiments. Number of sterile protected/challenged mice: 7×Luc (0/21), 1×CSP 6×Luc (1/20) and 1×7cPEAg (18/21). (b) Three and one day before sporozoite challenge, 1×7cPEAg immunized mice were injected with 400 μg of control (Ctr), CD4-depleting (a-CD4+, clone GK1.5) and CD8-depleting (a-CD8+, clone 2.43) monoclonal antibodies. GFP data comes from experiment showed in FIG. 11 (gray circles). Number of sterile protected/challenged mice: 7×Luc (0/7), 1×CSP 6×Luc (1/7) and 1×7cPEAg (ctr, 7/7; a-CD8, 0/7 and a-CD4, 7/7). Notice that depletion of CD8+ cells abolished sterile protection. *P<0.05, **P<0.01, ****P<0.0001 (ANOVA).

FIG. 16. Sterile protection induced by a multigenic combination in a single immunization dose. (a) Mice were immunized twice, four weeks apart, with 7× the individual dose (1 dose=5×10⁵ TU of non-concentrated VSV^{NJ} B2M LPs in the first immunization/1×10⁷ TU of non-concentrated VSV^{IND} B2M LPs in the second immunization) of the control antigen AL11-luciferase (Luc, black triangles), with the individual dose of CSP plus 6×Luc (CSP, black triangles), or with the individual dose of CSP and of 6 conserved PE antigens (11-05, 11-06, 11-07, 11-09, 11-10 and 18-10; black triangles; 2 im 7cPEAg). Alternatively, mice were administered only with the second individual immunization dose (1×10⁷ TU) of CSP and of 6 conserved PE antigens (11-05, 11-06, 11-07, 11-09, 11-10 and 18-10; grey diamonds; 1 im 7cPEAg). 4 weeks after the second immunization dose, mice were challenged with 5,000 GFP SPZs. The graph shows the individual log of parasitemia at day 5 post-challenge. Black bars are the average of log of parasitemia. Number of sterile protected/challenged mice: Luc (0/7), CSP (0/7), 2 im 7cPEAg (6/7) and 1 im 7cPEAg (6/7). (b) Mice were immunized once with 9× the individual dose (1 dose=1×10⁷ TU of non-concentrated VSV^{IND} B2M LPs) of the control antigen AL11-luciferase (Luc, black diamonds), or with the individual doses of CSP+ of 7 conserved PE antigens (11-05, 11-06, 11-07, 11-09, 11-10, 18-10, 30-03A and 30-03B; grey diamonds; 8cPEAg). Three and one day before sporozoite challenge, 8cPEAg immunized mice were injected with 400 μg of control (Ctr), CD4-depleting (a-CD4+, clone GK1.5) and CD8-depleting (a-CD8+, clone 2.43) monoclonal antibodies. 4 weeks after the single immunization dose, mice were challenged with 5,000 GFP SPZs. The graph shows the individual log of parasitemia at day 5 post-challenge. Black bars are the average of log of parasitemia. Number of sterile protected/challenged mice: 7×Luc (0/7) and 8cPEAg (ctr,6/7; a-CD8, 0/7 and a-CD4, 4/7). Notice that depletion of CD8+ cells abolished protection. *P<0.05; **P<0.01; ****P<0.0001; ns, P>0.05 (ANOVA).

FIG. 17. Sterile protection induced by a minimal combination of 5 PE antigens. (a) Mice were immunized twice, four weeks apart, with the individual dose multiplied by the number indicated in the circles (1 dose=5×10⁵ TU of non-concentrated VSV^{NJ} B2M LPs in the first immunization/1×10⁷ TU of non-concentrated VSV^{IND} B2M LPs in the second immunization). For example, for the control antigen AL11-luciferase (LUC), animals were immunized with 5× the individual dose. All groups received 5 doses, with exception of the positive control of protection that received 7 doses of LPs (7PEAg). 4 weeks after the second immunization dose, mice were challenged with 5,000 GFP SPZs. Bars represents the percentage of sterile protected mice. The numbers of sterile protected/challenged mice are shown at the right of bars. **P<0.01 (Fisher's Exact test). (b) Mice were immunized twice, four weeks apart, with the individual dose (1 dose=5×10⁵ TU of non-concentrated VSV^{NJ} B2M LPs in the first immunization/1×10⁷ TU of non-concentrated VSV^{IND} B2M LPs in the second immunization) of the control antigen GFP (GFP, black circles) or with the individual dose of CSP, TRAP, 18-10, 11-09 or 11-10 (grey triangles). Three and one day before sporozoite challenge, immunized mice were injected with 400 μg of control (Ctr), CD4-depleting (a-CD4+, clone GK1.5) and CD8-depleting (a-CD8+, clone 2.43) monoclonal antibodies. 4 weeks after the second immunization dose, mice were challenged with 5,000 GFP SPZs. Graphs show the average±sd of log of parasitemia at day 5 post-challenge. *P<0.05; ns, P>0.05 (ANOVA). (c) The 5 down-selected protective antigens were split according the presence of predicted CD8 T cell epitopes and respecting conserved structural domains as depicted by the schematic representation of the antigens. The graphs above the schematic proteins represent the distribution of epitopes predicted to bind to H2Kb (8 aa) and H2Kd (9 aa) MHC class I molecules using SYFPEITHI (score) and IEDB ANN IC 50 (nM). The graphs on the left of schematic proteins represent the protection induced by these constructs, where bars are the average±sd of log of parasitemia at day 5 post-challenge. Data shown for antigen 11-09 come from FIG. 11. Dotted line represents the inferior limit of the tolerance interval of the control calculated in the FIG. 11. *P<0.05; ns, P>0.05 (ANOVA). (d) Correlation of the best epitope predicted to bind to MHC class I molecules in the segments of CD8+ T cell dependent PE antigens and mean protective activity obtained from 17c. Circles show the IC50 using IEDB ANN software and squares the score values using SYFPEITHI. Dotted line shows the average of Luc control.

FIG. 18. Clustering of CD8 T cell epitopes in conserved amino acid regions and binding of predicted Pf epitopes to HLA A02:01. Amino acid sequences of protective orthologous antigens from rodent-infecting P. berghei, macaque-infecting P. cynomolgi, and human-infecting P. falciparum and P. vivax parasites were aligned by Multiple Sequence Comparison by Log-Expectation (MUSCLE). Vertical black bars represent identical amino acids conserved in the four plasmodial species. The graph shows the distribution of Pb epitopes predicted to bind to H2Kb (8 aa) and H2Kd (9 aa) MHC class I molecules or of Pf epitopes predicted to bind to the HLA A02:01 (9 mers) using SYFPEITHI (score) and IEDB ANN IC50 (nM). The best predicted HLA binders were tested in the assay of stabilization of MHC class I molecule in the presence of peptide and β2-microglobulin (REVEAL® Score). The score of 100 corresponds to the binding of a positive control peptide. Notice the clustering of epitopes in regions of conserved amino acids.

FIG. 19. plasmid used to produce VSV-pseudotyped lentiviral particles: pTRIP CMV GFP

The sequence of the plasmid is constituted by the following functional regions wherein the cis-active lentiviral regions are derived from the HIV genome, and the promoter driving the expression of the protein (GFP) is CMV:

The insert in the plasmid that provides the vector genome is composed as follows: LTR-Ψ-RRE-cPPT/CTS-CMV-GFP-WPRE-ΔU3LTR, wherein
LTR is Long Terminal Repeat
Psi (Ψ) is Packaging signal
RRE is Rev Responsive Element
CMV is Immediate early CytoMegaloVirus promoter
CPPT is central PolyPurine Tract, and wherein the nucleotide segment from
cPPT to CTS forms the flap sequence
CTS is Central Termination Sequence
WPRE is Woodchuck hepatitis virus Post Regulatory Element
   The nucleotide sequence is provided as SEQ ID No. 1

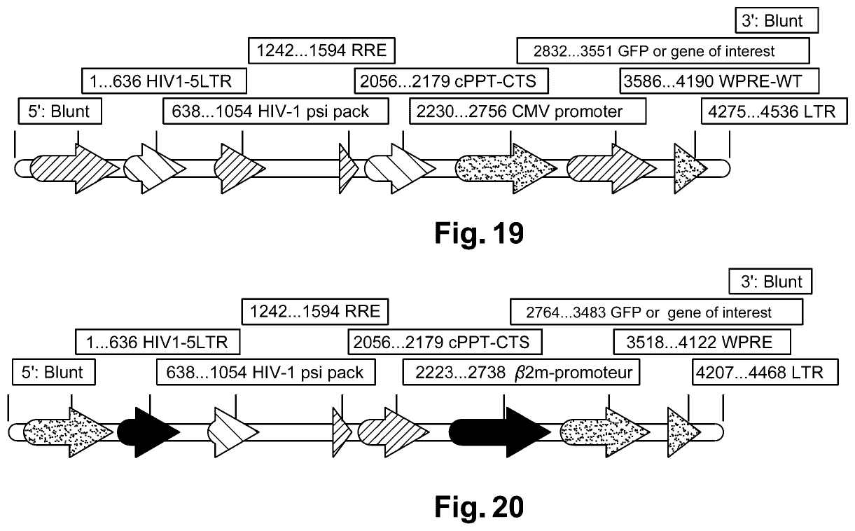

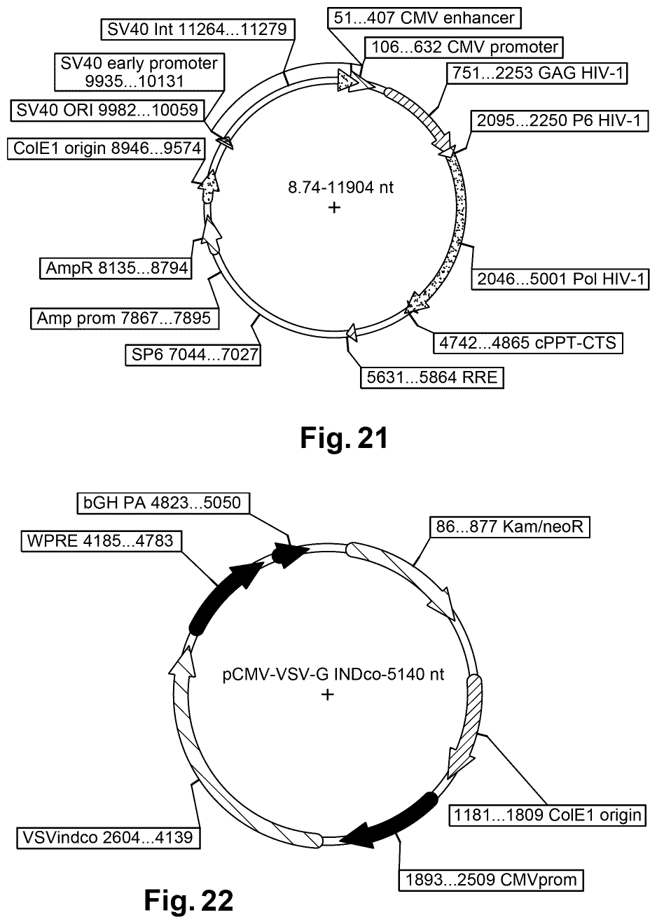

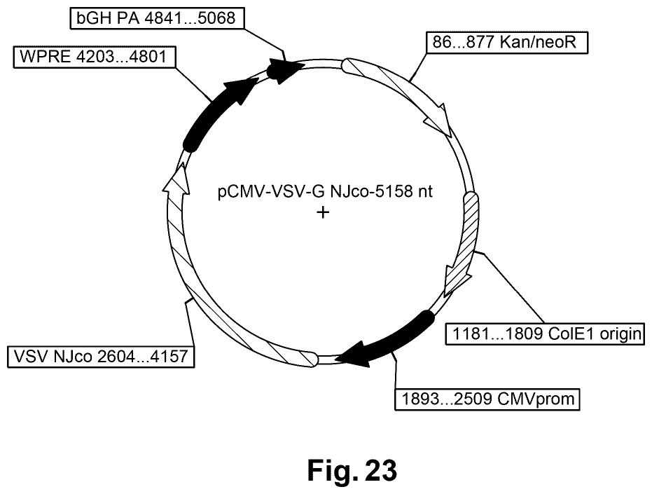

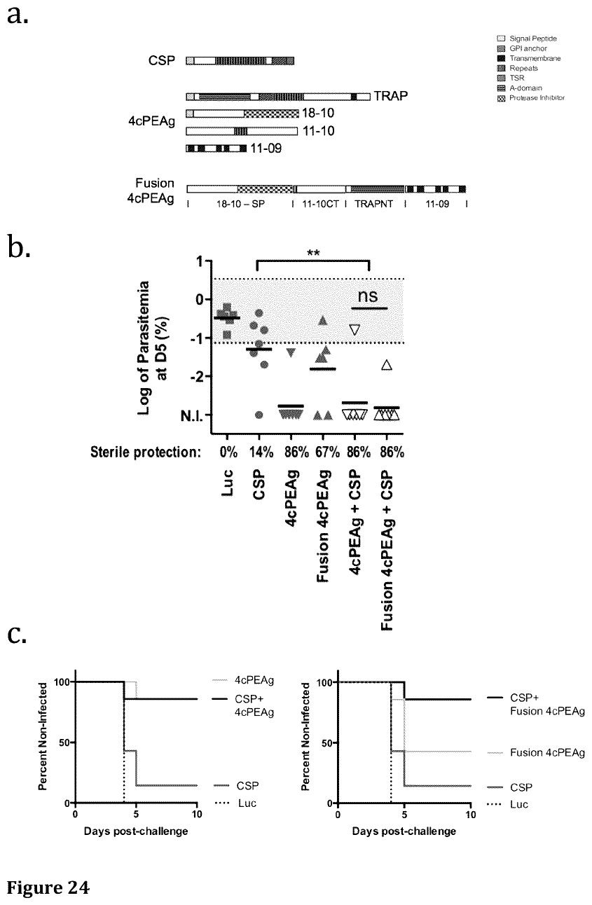

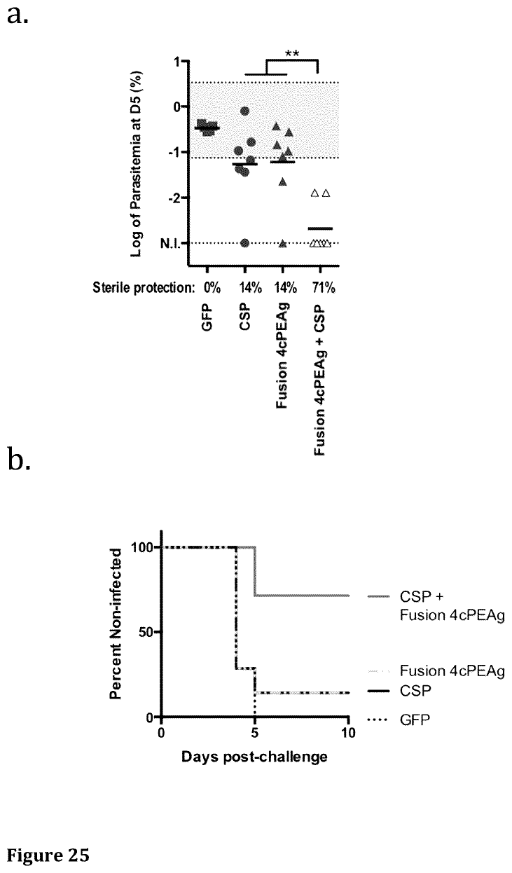

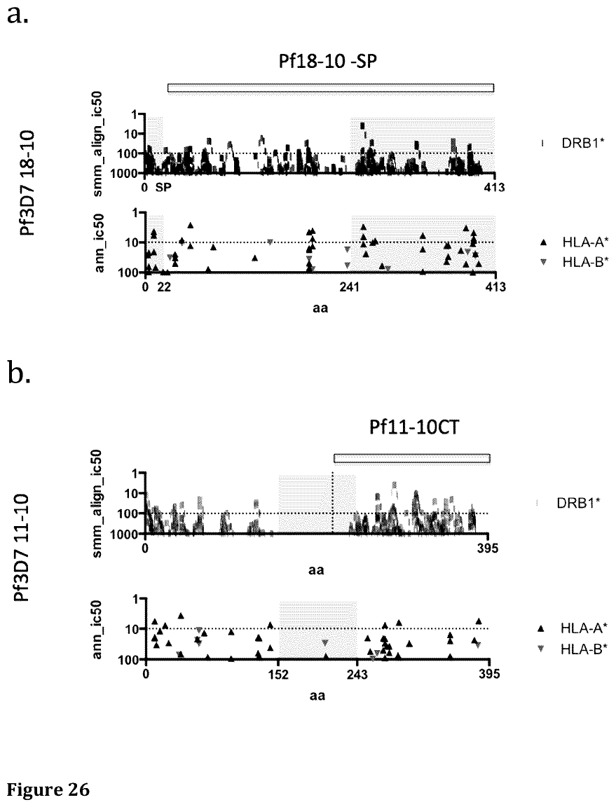

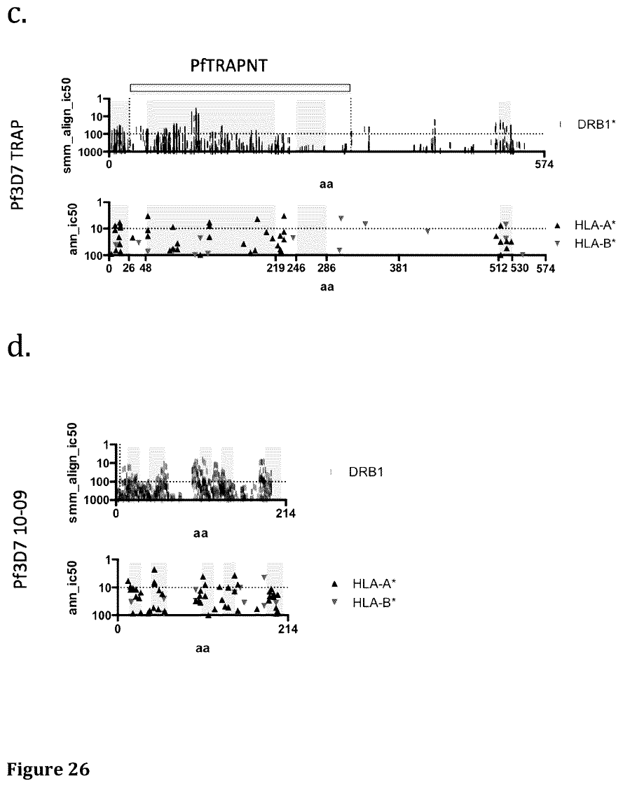

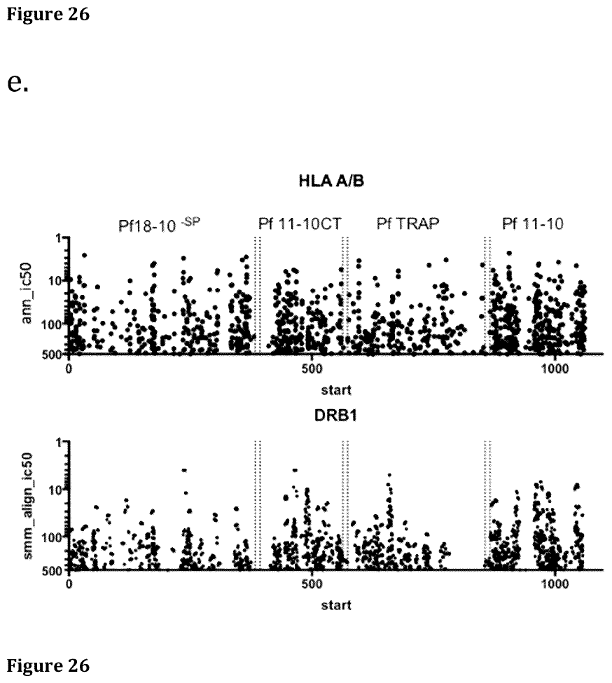

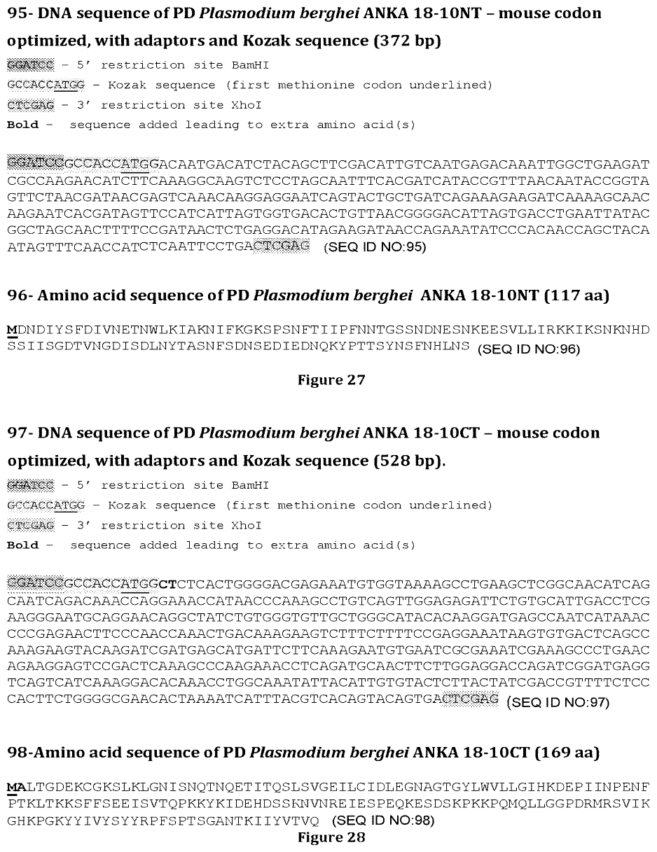

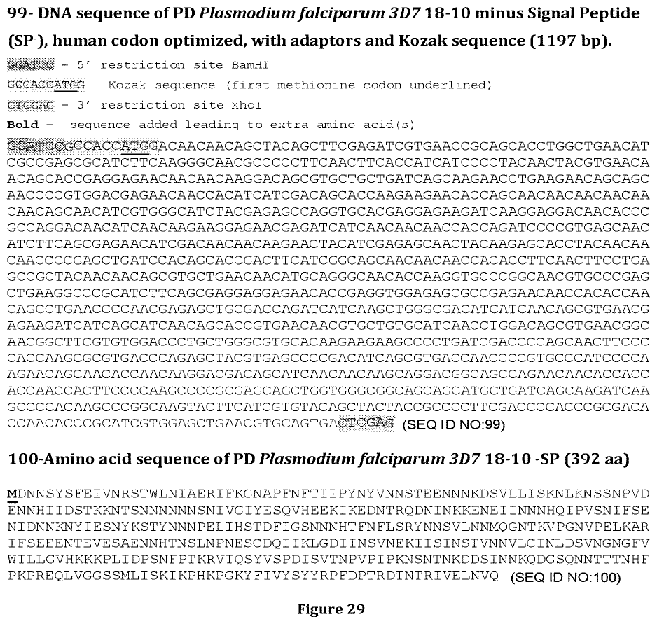

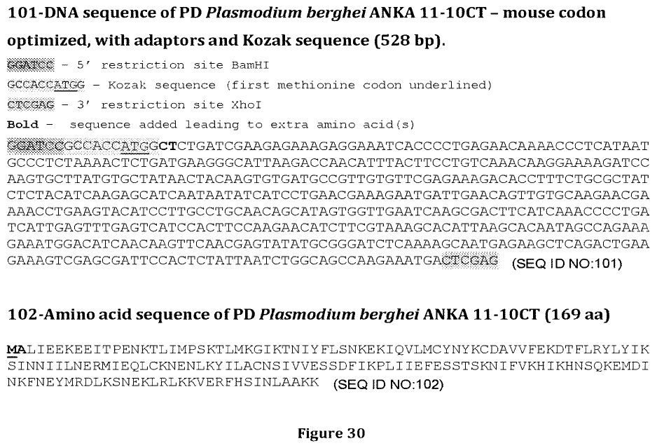

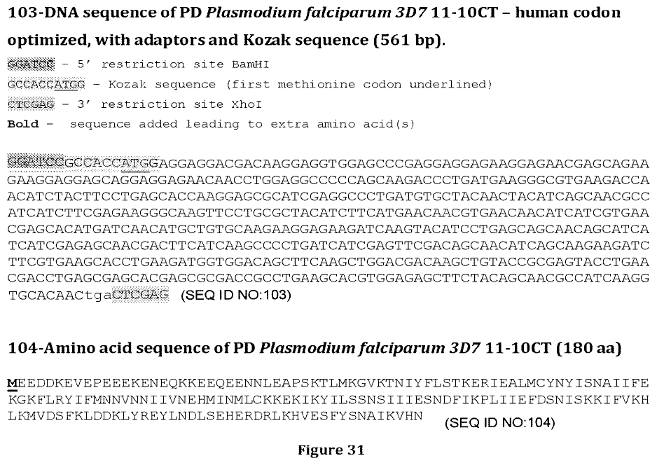

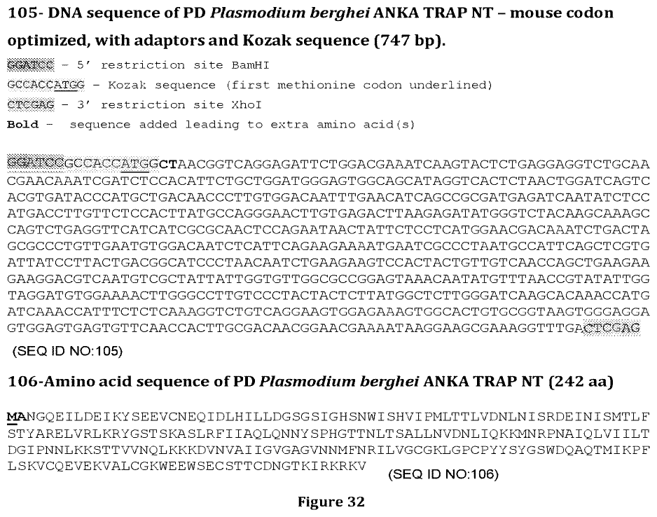

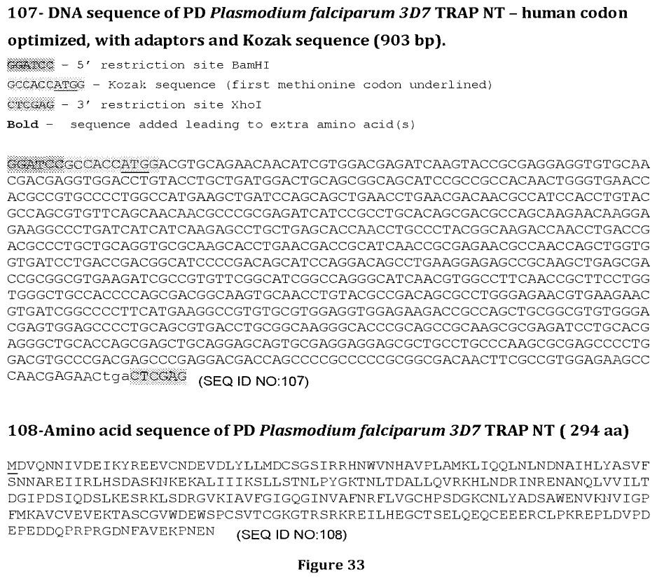

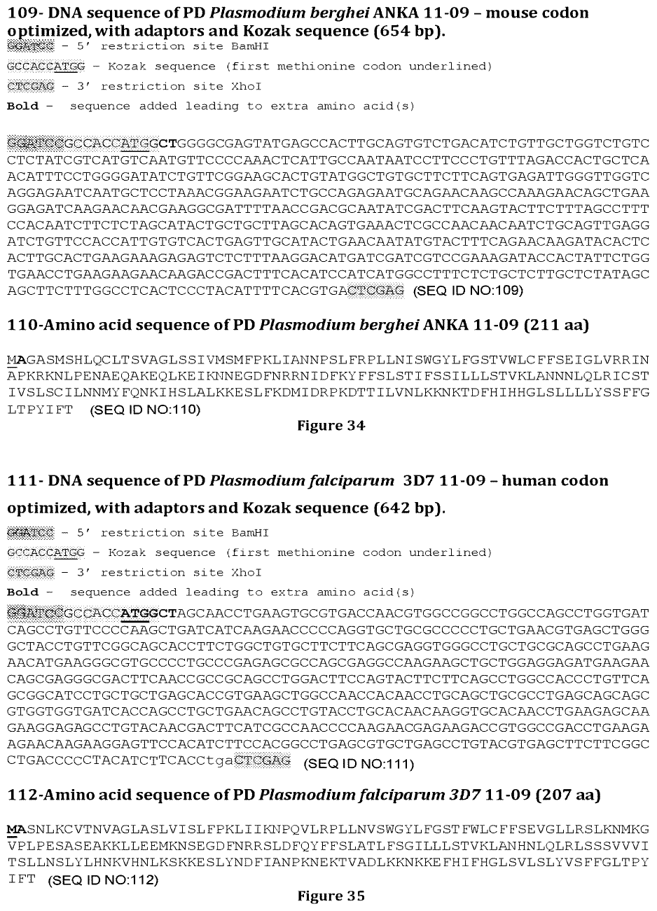

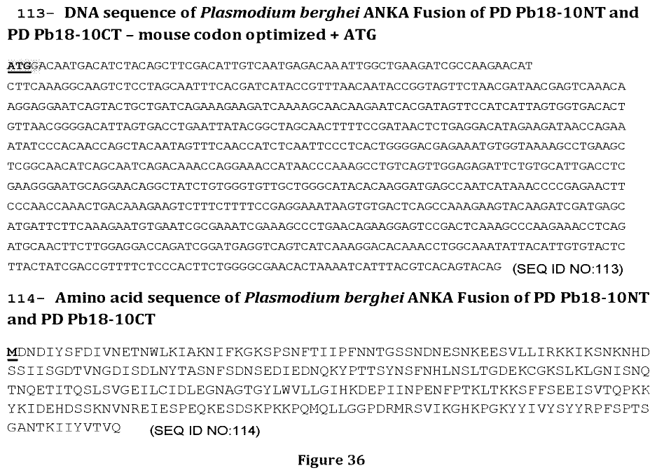

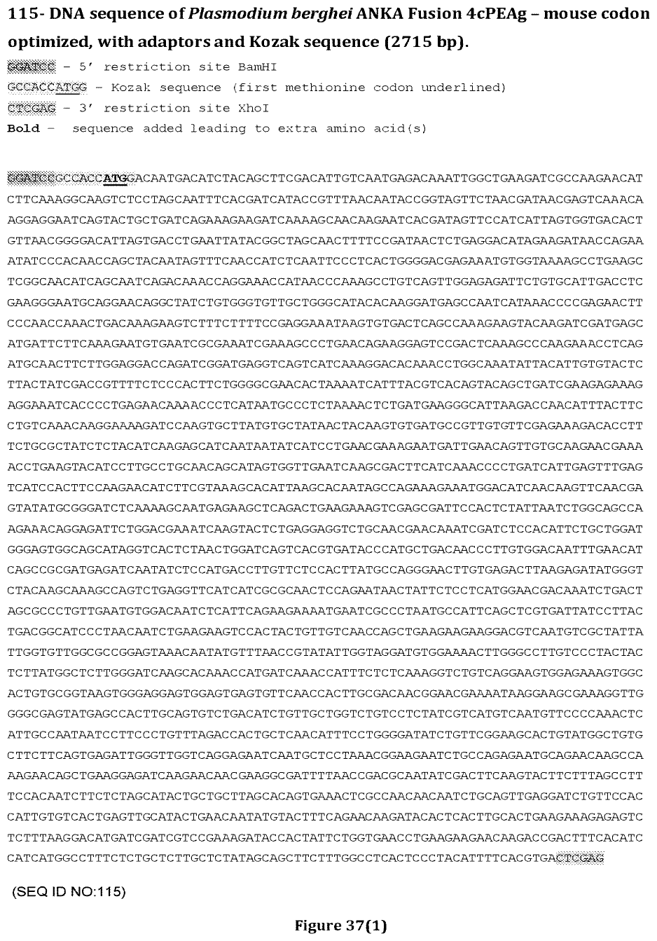

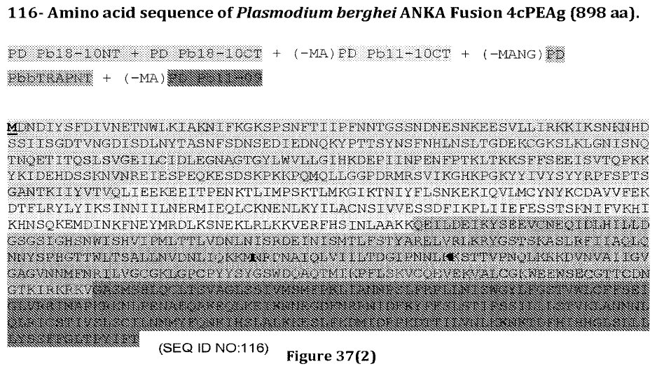

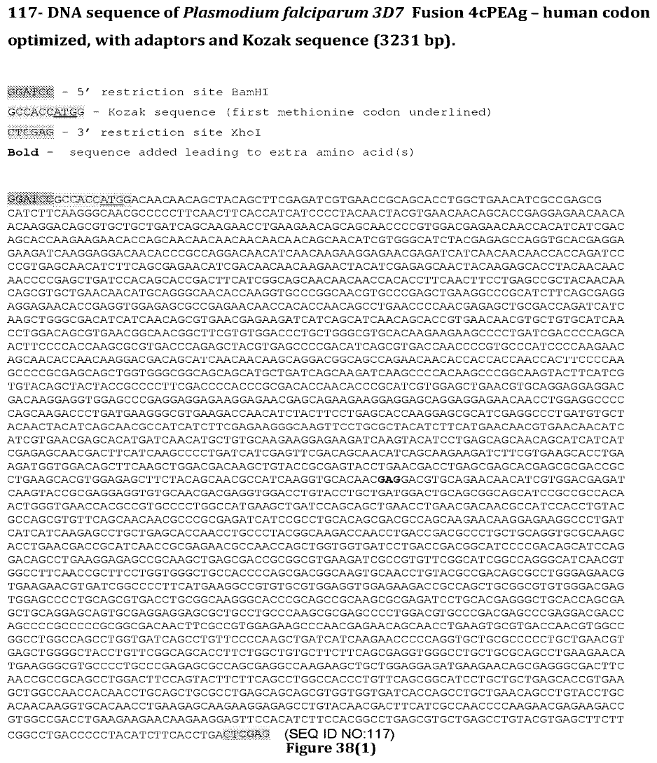

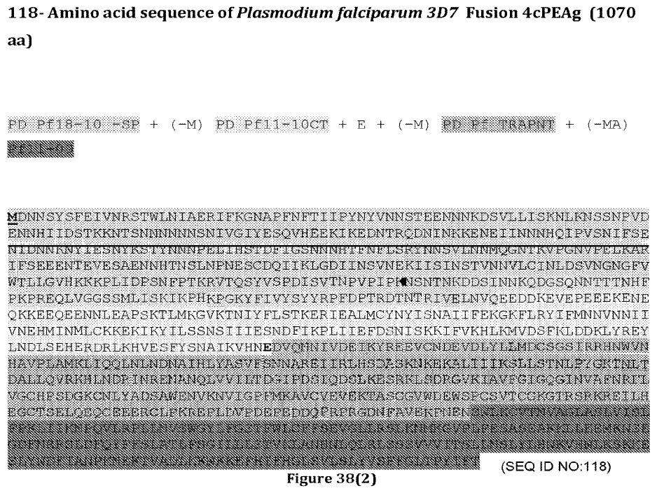

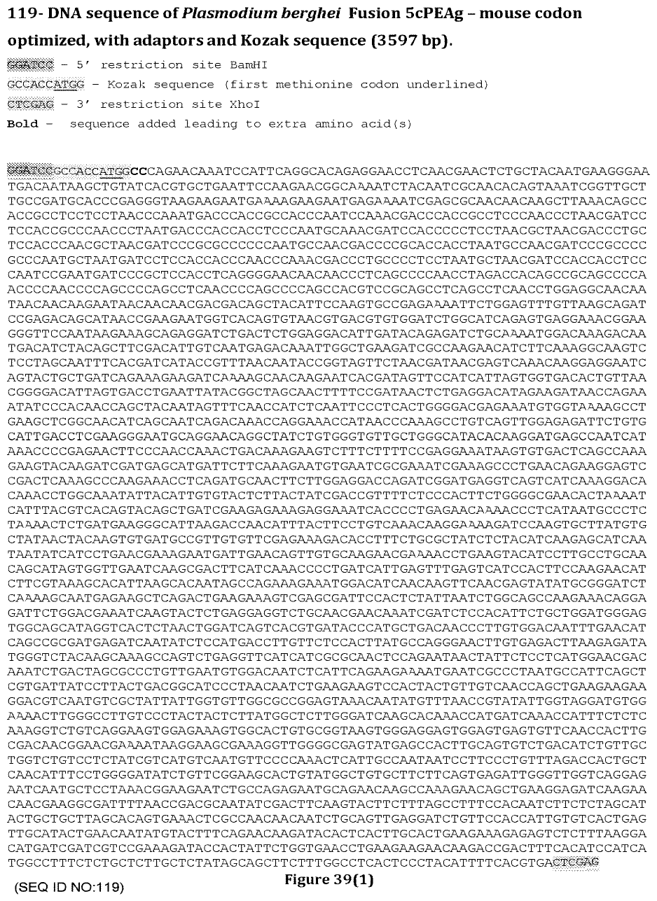

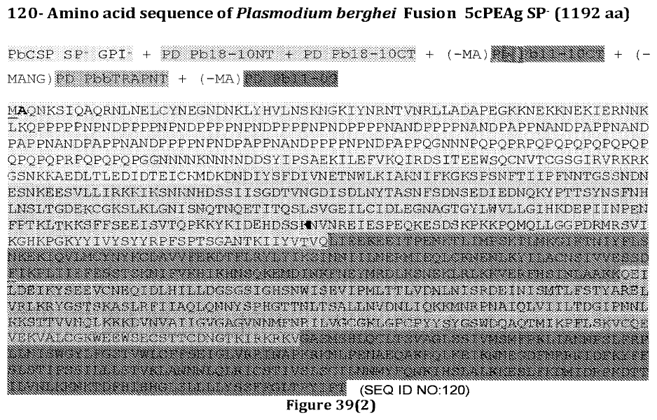

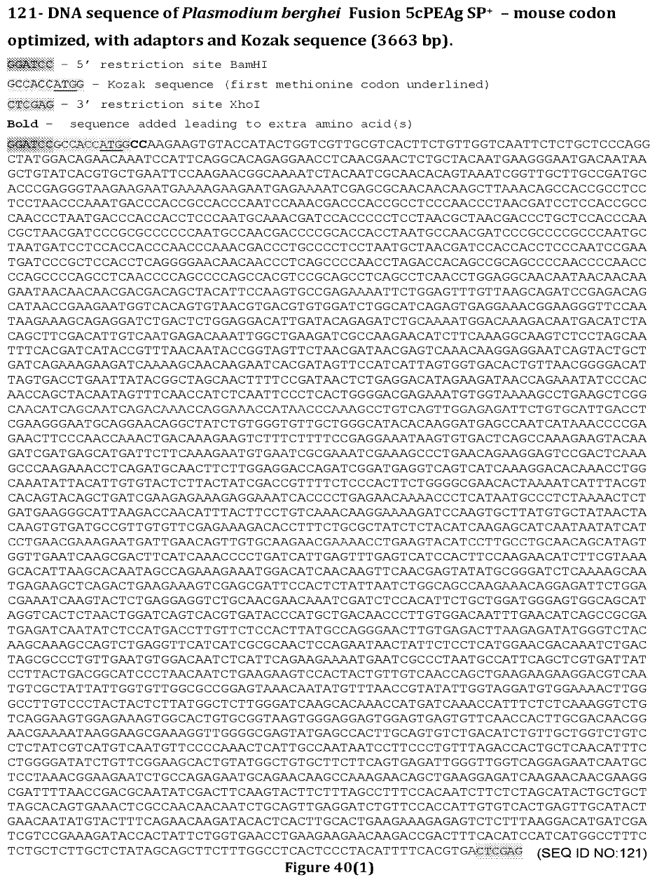

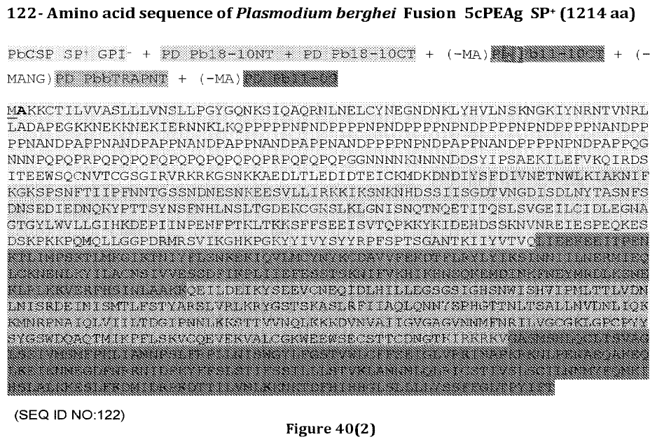

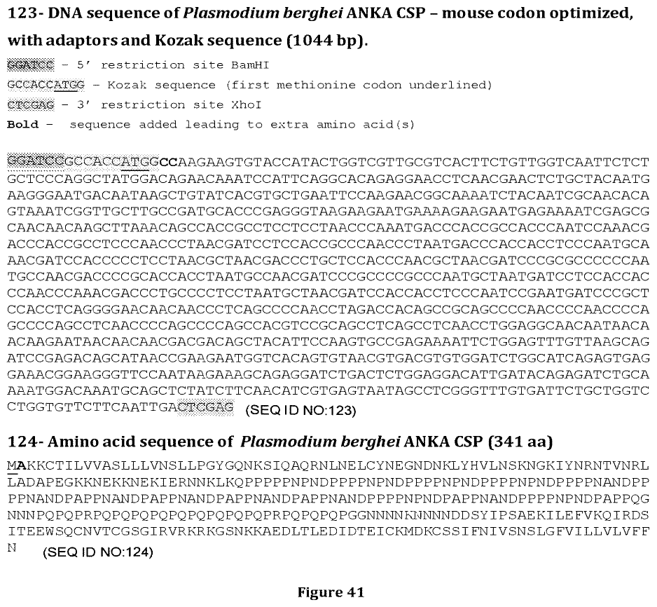

FIG. 20: alternative plasmid (to the plasmid of FIG. 19) used to produce VSV-pseudotyped lentiviral particles: pTRIP B2M GFP

The insert in the plasmid that provides the vector genome is composed as follows: LTR—Y-RRE-cPPT/CTS-B2M-GFP-WPRE-ΔU3LTR.

The nucleotide sequence is provided as SEQ ID No. 2.

FIG. 21. plasmid used to produce VSV-pseudotyped lentiviral particles: packaging 8.74 plasmid

The plasmid provides the required GAG and POL coding sequences of the HIV-1 lentivirus under the control of the CMV promoter.
The nucleotide sequence is provided as SEQ ID No. 3.

FIG. 22. plasmid used to produce VSV-pseudotyped lentiviral particles: encapsidation plasmid pCMV-VSV-INDco

The envelope protein is the VSV-G of the Indiana strain and the coding sequence has been mouse-codon optimized.
The nucleotide sequence is provided as SEQ ID No. 4.

FIG. 23. alternative plasmid (to plasmid of FIG. 22) used to produce VSV-pseudotyped lentiviral particles: encapsidation plasmid pCMV-VSV-NJco The envelope protein is the VSV-G of the New-Jersey strain and the coding sequence has been mouse-codon optimized.

The nucleotide sequence is provided as SEQ ID No. 5.

FIG. 24. Sterile protection induced by two immunization doses of the Fusion 4cPEAg+CSP.
(a) Scheme of antigens used in the experimental groups. CSP refers to the PbCSP. 4cPEAg refers to the combination of PbTRAP, antigen Pb18-10, antigen Pb11-10 and antigen Pb11-09. Fusion 4cPEAg refers to the chimeric antigen formed by the fuse of the antigen Pb18-10 without its signal peptide (SP), followed by the protective domains 11-10 CT and TRAPNT, and the antigen Pb11-09. GPI (glycosylphosphatidylinositol), TSR (thrombospondin type I repeat).
(b) Four weeks-old C57BL/6 mice (n=6-7 per group) were acclimated for 3 weeks and immunized intraperitoneally with 5×10⁵ TU/antigen using non-concentrated VSV^{NJ} B2M LPs. Four weeks after the first immunization, all groups received intraperitoneally 3×10⁷ TU per respective antigen of concentrated VSV^{IND} B2M LPs. Four weeks after the second immunization, mice were challenged with the microinjection of 5,000 GFP PbSPZs in the footpad. The graph shows the log of parasitemia of individual mouse at day 5 post-challenge. Traces represent the mean of the log parasitemia. The superior and inferior dotted lines delineate the 95% tolerance interval (grey box) of the control group established in the experiment of FIG. 11. NI (not infected). Sterile protection indicates the percentage of mice with no detectable parasitemia at day 10 post-challenge.
(c) Profile of protection of challenged mice.

FIG. 25. Sterile protection induced by a single immunization doses of the Fusion 4cPEAg+CSP.
(a) Four weeks-old C57BL/6 mice (n=5-7 per group) were acclimated for 3 weeks and immunized intraperitoneally with 1×10⁷ TU of GFP VSV^{NJ} B2M LPs or 1×10⁷ TU of CSP VSV^{IND} B2M LPs, in the presence or not of 4×10/TU of Fusion 4cPEAg VSV^{IND} B2M LPs. Four weeks after the first immunization, mice were challenged with the microinjection of 5,000 GFP PbSPZs in the footpad. The graph shows the log of parasitemia of individual mouse at day 5 post-challenge. Traces represent the mean of the log parasitemia. The superior and inferior dotted lines delineate the 95% tolerance interval (grey box) of the control group established in the experiment of FIG. 11. NI (not infected). Sterile protection indicates the percentage of mice with no detectable parasitemia at day 10 post-challenge.
(b) Profile of protection of challenged mice.

FIG. 26. P. falciparum 4cPEAg fusion.

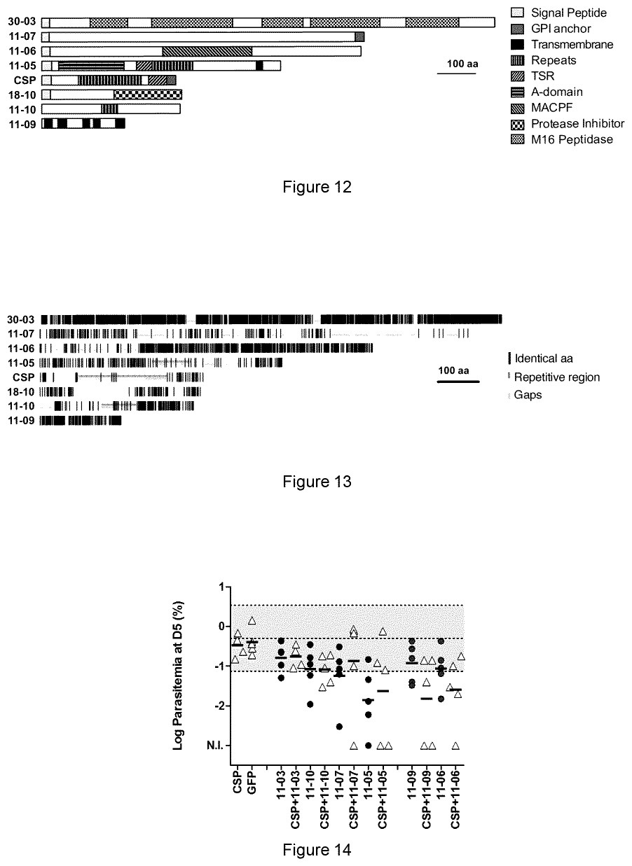

Epitopes from the Pf 4cPEAgs predicted to bind to the Human Leukocyte Antigen (HLA) were identified using the immune epitope database (iedb; www.iedb.org). (a-c) Bars represent epitopes predicted to bind on the HLA-DRB1*01:01, *03:01, *04:01, *04:05, *07:01, *08:02, *09:01, *11:01, *12:01, *13:02 and *15:01. Triangles represent epitopes predicted to bind to the HLA A*01:01, *02:01, *02:03, *02:06, *03:01, *11:01, *23:01,*24:02, *26:01, *30:01, *30:02, *31:01, *32:01, *33:01, *68:01 and *68:02. Inverted triangles represent epitopes predicted to bind to the HLA-B*07:02, *08:01, *15:01, *35:01, *40:01, *44:02, *44:03, *51:01, *53:01, *57:01 and *58:01. White horizontal bars represent the regions used to design the Pf 4cPEAg fusion based on the content of class I and II predicted epitopes and structural/sequence similarity with the protective domains tested using P. berghei. Gray shadows represent conserved structural domains depicted in the FIGS. 12 and 24a. SP, signal peptide. Antigens are (a) Pf18-10, (b) Pf11-10, (c) PfTRAP, (d) Pf11-09. (e) Selected regions of the Pf 4cPEAgs (white bars) were chimerized generating the Pf 4cPEAg Fusion. The dotted lines represent the junction between two adjacent antigens/protective domains and show the absence of formation of neo-epitopes.

FIGS. 27 to 41: FIGS. 27 to 41 describe the DNA and respective amino acid sequences of the nucleic acids and polypeptides disclosed in the Table that follows under SEQ ID No. 95 to 124. The figures show the Kozak sequence (nucleotides 1-10 of SEQ ID NO:84), BamHI site (SEQ ID NO:86), and XhoI site (SEQ ID NO:87).

The following table provides the list and identification of the sequences contained in the sequence listing.

| SEQ ID | | | |
|---|---|---|---|
| No. | Sequence designation | Origin | Type |
| 1 | pTRIP CMV GFP | | DNA |
| 2 | pTRIP B2M GFP | | DNA |
| 3 | PACKAGING 8.74 PLASMID | | DNA |
| 4 | pCMV-VSV-INDco | | DNA |
| 5 | pCMV-VSV-Njco | | DNA |
| 6 | eGFP | | DNA |
| 7 | eGFP protein | | protein |
| 8 | AL11-Luciferase | | protein |
| 9 | AL11-Luciferase | | protein |
| 10 | circumsporozoite (CS) protein | P. berghei | DNA |
| | (CSP) mouseCO + Kozak | ANKA strain | |
| 11 | PbCSP (mouseCO + Kozak) | P. berghei | protein |
| | | ANKA strain | |
| 12 | PbCSP | P. berghei | protein |
| | | ANKA strain | |
| 13 | PfCSP humanCO + Kozak | P falciparum | DNA |
| | | 3D7 strain | |
| 14 | PfCSP (humanC0 + Kozak) | P falciparum | protein |
| | | 3D7 strain | |
| 15 | PfCSP | P falciparum | protein |
| 16 | PvCSP humanCO + Kozak | P vivax Sal-1 | DNA |
| | | strain | |
| 17 | PvCSP (humanCO + Kozak) | P vivax Sal-1 | protein |
| | | strain | |
| 18 | PvCSP | P vivax Sal-1 | protein |
| | | strain | |
| 19 | thrombospondin-related | P. berghei | DNA |
| | anonymous protein | ANKA strain | |
| | (PbTRAP) mouseCO + Kozak | | |
| 20 | PbTRAP (mouseCO + Kozak) | P. berghei | protein |
| | | ANKA strain | |
| 21 | PbTRAP | P. berghei | protein |
| | | ANKA strain | |
| 22 | PfTRAP humanCO + Kozak | P falciparum | DNA |
| | | 3D7 strain | |
| 23 | PfTRAP (humanCO + Kozak) | P falciparum | protein |
| | | 3D7 strain | |
| 24 | PfTRAP | P falciparum | protein |
| 25 | PvTRAPhumanCO | P vivax Sal-1 | DNA |
| | | strain | |
| 26 | PvTRAP | P vivax Sal-1 | protein |
| | | strain | |
| 27 | PvTRAP | P vivax | protein |
| 28 | inhibitor of cysteine proteases (ICP) | P. berghei | DNA |
| | mouseCO + Kozak | ANKA strain | |
| 29 | PbICP (mouseCO + Kozak) | P. berghei | protein |
| | | ANKA strain | |
| 30 | PbICP | P. berghei | protein |
| | | ANKA strain | |
| 31 | PfICP humanCO | P falciparum | DNA |
| | | 3D7 strain | |
| 32 | PfICP | P falciparum | protein |
| | | 3D7 strain | |
| 33 | PfICP | P falciparum | protein |
| 34 | PvICP humanCO + Kozac | P vivax Sal-1 | DNA |
| | | strain | |
| 35 | PvICP (humanCO + Kozac) | P vivax Sal-1 | protein |
| | | strain | |
| 36 | PvICP | P vivax | protein |
| 37 | Bergheilysin-A-mouseCO + | P. berghei | DNA |
| | Kozak | ANKA strain | |
| 38 | Bergheilysin-A (1-777, | P. berghei | protein |
| | mouse CO + Kozak) | ANKA strain | |
| 39 | Bergheilysin entire ORF (1-1149) | P. berghei | protein |
| | | ANKA strain | |
| 40 | Falcilysin human CO + Kozak | P falciparum | DNA |
| | | 3D7 strain | |
| 41 | Falcilysin (human CO + Kozak) | P falciparum | protein |
| | | 3D7 strain | |
| 42 | Falcilysin | P falciparum | protein |
| | | 3D7 strain | |
| 43 | PvFalcilysin human CO + Kozak | P vivax Sal-1 | DNA |
| | | strain | |
| 44 | PvFalcilysin (humanCO + Kozak) | P vivax Sal-1 | protein |
| | | strain | |
| 45 | PvFalcilysin | P vivax Sal-1 | protein |
| | | strain | |
| 46 | Bergheilysin-B- mouseCO + | P. berghei | DNA |
| | Kozak + signal peptide (SP) | ANKA strain | |
| 47 | Bergheilysin-B (SP + 778-1149, | P. berghei | protein |
| | mouse CO + Kozak) | ANKA strain | |
| 48 | perforin like protein 1 (SPECT2) | P. berghei | DNA |
| | mouseCO + Kozak | ANKA strain | |
| 49 | PbSPECT2 (mouseCO + Kozak) | P. berghei | protein |
| | | ANKA strain | |
| 50 | PbSPECT2 | P. berghei | protein |
| | | ANKA strain | |
| 51 | PfSPECT2 human CO + Kozak | P falciparum | DNA |
| | | 3D7 strain | |
| 52 | PfSPECT2 (humanCO + Kozak) | P falciparum | protein |
| | | 3D7 strain | |
| 53 | PfSPECT2 | P falciparum | protein |
| 54 | PvSPECT2 human CO + Kozak | P vivax Sal-1 | DNA |
| | | strain | |
| 55 | PvSPECT2 (human CO + Kozak) | P vivax Sal-1 | protein |
| | | strain | |
| 56 | PvSPECT2 | P vivax | protein |
| 57 | GPI_P113 mouseCO + Kozak | P. berghei | DNA |
| | | ANKA strain | |
| 58 | Pb GPI_P113 (mouseCO + Kozak) | P. berghei | protein |
| | | ANKA strain | |
| 59 | Pb GPI_P113 | P. berghei | protein |
| | | ANKA strain | |
| 60 | PfP113 human CO + Kozak | P falciparum | DNA |
| | | 3D7 strain | |
| 61 | PfP113 (human CO + Kozak) | P falciparum | protein |
| | | 3D7 strain | |
| 62 | P113 | P falciparum | protein |
| 63 | PvP113 human CO + Kozak | P vivax Sal-1 | DNA |
| | | strain | |
| 64 | PvP113 (human CO + Kozak) | P vivax Sal-1 | protein |
| | | strain | |
| 65 | P113 | P vivax | protein |
| 66 | PbAg40 mouse CO + Kozak | P. berghei | DNA |
| | | ANKA strain | |
| 67 | PbAg40 (mouse CO + Kozak) | P. berghei | protein |
| | | ANKA strain | |
| 68 | PbAg40 | P. berghei | protein |
| | | ANKA strain | |
| 69 | PfAg40 human CO + Kozak | P falciparum | DNA |
| | | 3D7 strain | |
| 70 | PfAg40 (human CO + Kozak) | P falciparum | protein |
| | | 3D7 strain | |
| 71 | Ag40 | P falciparum | protein |
| 72 | PvAg40 human CO + Kozak | P vivax Sal-1 | DNA |
| | | strain | |
| 73 | PvAg40 (human CO + Kozak) | P vivax Sal-1 | protein |
| | | strain | |
| 74 | PvAg40 | P vivax Sal-1 | protein |
| | | strain | |
| 75 | PbAg45 mouse CO + Kozak | P. berghei | DNA |
| | | ANKA strain | |
| 76 | PbAg45 (mouse CO + Kozak) | P. berghei | protein |
| | | ANKA strain | |
| 77 | PbAg45 | P. berghei | protein |
| | | ANKA strain | |
| 78 | PfAg45 human CO + Kozak | P falciparum | DNA |
| | | 3D7 strain | |
| 79 | PfAg45 (human CO + Kozak) | P falciparum | protein |
| | | 3D7 strain | |
| 80 | PfAg45 | P falciparum | protein |
| 81 | PvAg45 human CO + Kozak | P vivax Sal-1 | DNA |
| | | strain | |
| 82 | PvAg45 (human CO + Kozak) | P vivax Sal-1 | protein |
| | | strain | |
| 83 | PvAg45 | P vivax | protein |
| 84 | Kozak consensus sequence | | DNA |
| 85 | Kozak consensus sequence | | DNA |
| 86 | BamHI site | | DNA |
| 87 | Xhol site | | DNA |
| 88-94 | CD8 T cell epitopes | | protein |

DNA and amino acid sequences used in the chimeric fusion antigenic polypeptides for P. berghei and P. falciparum

| SEQ | |
|---|---|
| ID | |
| N. | Description of sequence |
| 95 | DNA sequence of PD Plasmodium berghei ANKA 18-10NT— |
| | mouse codon optimized, with adaptors and Kozak sequence |
| | (372 bp) |
| 96 | Amino acid sequence of PD Plasmodium berghei ANKA |
| | 18-10NT (117 aa) |
| 97 | DNA sequence of PD Plasmodium berghei ANKA 18-10CT— |
| | mouse codon optimized, with adaptors and Kozak sequence |
| | (528 bp). |
| 98 | Amino acid sequence of PD Plasmodium berghei ANKA |
| | 18-10CT (169 aa) |
| 99 | DNA sequence of PD Plasmodium falciparum 3D7 18-10 |
| | minus Signal Peptide (SP⁻), human codon optimized, with |
| | adaptors and Kozak sequence (1197 bp). |
| 100 | Amino acid sequence of PD Plasmodium falciparum 3D7 |
| | 18-10-SP (392 aa) |
| 101 | DNA sequence of PD Plasmodium berghei ANKA 11-10CT— |
| | mouse codon optimized, with adaptors and Kozak sequence |
| | (528 bp). |
| 102 | Amino acid sequence of PD Plasmodium berghei ANKA |
| | 11-10CT (169 aa) |
| 103 | DNA sequence of PD Plasmodium falciparum 3D7 11-10CT— |
| | human codon optimized, with adaptors and Kozak sequence |
| | (561 bp). |
| 104 | Amino acid sequence of PD Plasmodium falciparum 3D7 |
| | 11-10CT (180 aa) |
| 105 | DNA sequence of PD Plasmodium berghei ANKA TRAP NT— |
| | mouse codon optimized, with adaptors and Kozak sequence |
| | (747 bp). |
| 106 | Amino acid sequence of PD Plasmodium berghei ANKA |
| | TRAP NT (242 aa) |
| 107 | DNA sequence of PD Plasmodium falciparum 3D7 TRAP |
| | NT—human codon optimized, with adaptors and Kozak |
| | sequence (903 bp). |
| 108 | Amino acid sequence of PD Plasmodium falciparum 3D7 |
| | TRAP NT (294 aa) |
| 109 | DNA sequence of PD Plasmodium berghei ANKA 11-09— |
| | mouse codon optimized, with adaptors and Kozak sequence |
| | (654 bp). |
| 110 | Amino acid sequence of PD Plasmodium berghei ANKA |
| | 11-09 (211 aa) |
| 111 | DNA sequence of PD Plasmodium falciparum 3D7 11-09— |
| | human codon optimized, with adaptors and Kozak sequence |
| | (642 bp). |
| 112 | Amino acid sequence of PD Plasmodium falciparum 3D7 |
| | 11-09 (207 aa) |
| 113 | DNA sequence of Plasmodium berghei ANKA Fusion of |
| | PDPb18-10NT and PD Pb18-10CT—mouse codon |
| | optimized + ATG (852pb) |
| 114 | Amino acid sequence of Plasmodium berghei ANKA Fusion |
| | of PD Pb18-10NT and PD Pb18-10CT (284aa) |
| 115 | DNA sequence of Plasmodium berghei ANKA Fusion |
| | 4cPEAg—mouse codon optimized, with adaptors and |
| | Kozak sequence (2715 bp). |
| 116 | Amino acid sequence of Plasmodium berghei ANKA Fusion |
| | 4cPEAg (898 aa). |
| 117 | DNA sequence of Plasmodium falciparum 3D7 Fusion |
| | 4cPEAg—human codon optimized, with adaptors and |
| | Kozak sequence (3234 bp). |
| 118 | Amino acid sequence of Plasmodium falciparum 3D7 |
| | Fusion 4cPEAg (1070 aa) |
| 119 | DNA sequence of Plasmodium berghei Fusion 5cPEAg— |
| | mouse codon optimized, with adaptors and Kozak |
| | sequence (3597 bp). |
| 120 | Amino acid sequence of Plasmodium berghei Fusion |
| | 5cPEAg SP⁻ (1192 aa) |
| 121 | DNA sequence of Plasmodium berghei Fusion |
| | 5cPEAg SP⁺—mouse codon optimized, with adaptors |
| | and Kozak sequence (3663 bp). |
| 122 | Amino acid sequence of Plasmodium berghei Fusion |
| | 5cPEAg SP⁺ (1214 aa) |
| 123 | DNA sequence of Plasmodium berghei ANKA CSP— |
| | mouse codon optimized, with adaptors and Kozak |
| | sequence (1044 bp). |
| 124 | Amino acid sequence of Plasmodium berghei ANKA |
| | CSP (341 aa) |

Additional information relating to some of the sequences disclosed in the above table are provided in the table below.

| SEQ ID | GenBank | strain | pubmed |
|---|---|---|---|
| 15 | BAM84930.1 | Plasmodium falciparum isolate | 23295064 |
| | | Pal97-042 origin: Philippines | |
| | ACO49323 | Plasmodium falciparum″ isolate | 19460323 |
| | | A5 origin: Thailand | |
| 18 | AAA29535.1 | P. vivax (strain Thai; isolate | 2290443 |
| | | NYU Thai) origin: Thailand | |
| 24⁽¹⁾ | EWC74605.1 | Plasmodium falciparum UGT5.1 | |
| | | strain origin: Uganda | |
| 27 | AIU97014.1 | Plasmodium vivax isolate = | |
| | | “TMS38” origin: Thailand | |
| 36⁽³⁾ | KMZ87332.1 | Plasmodium vivax Brazil I strain | |
| 56 | KMZ82648.1 | Plasmodium vivax India VII | |
| 65 | KMZ78214.1 | Plasmodium vivax India VII | |
| 83 | KMZ90984.1 | Plasmodium vivax Mauritania I | |

⁽¹⁾https://www.ncbi.nlm.nih.govhiosample/SAMN01737342
⁽²⁾https://www.ncbi.nlm.nih.gov/biosample/SAMEA2394724
⁽³⁾https://www.ncbi.nlm.nih.gov/biosample/SAMN00710434

### EXAMPLES

To approach the complex problem of identifying protective antigens, the inventors devised a functional screening to identify and combine novel PE protective antigens using a rodent malaria model where mice (C57BL/6) are extremely susceptible to Plasmodium berghei (Pb) sporozoite infection. In this model, sterilizing protection induced by live irradiated sporozoites is mediated by antibodies and mainly by CD8 T cell responses against sporozoites and liver stages, respectively. The inventors' screening strategy was designed based on four main features: i) parameterized selection of 55 PE antigens based on abundance, orthology, predicted topology and function, ii) synthesis of codon-optimized antigens to avoid AT-rich plasmodial sequences and maximize the expression in mammalian cells, iii) immunization using HIV-based lentiviral vector that elicits strong humoral and cellular responses^{11,12}, and iv) measurement of protection after a stringent challenge of sporozoites inoculated sub-cutaneously in the immunized mice.

### 1. Setting Up the Parameters of the Screening.

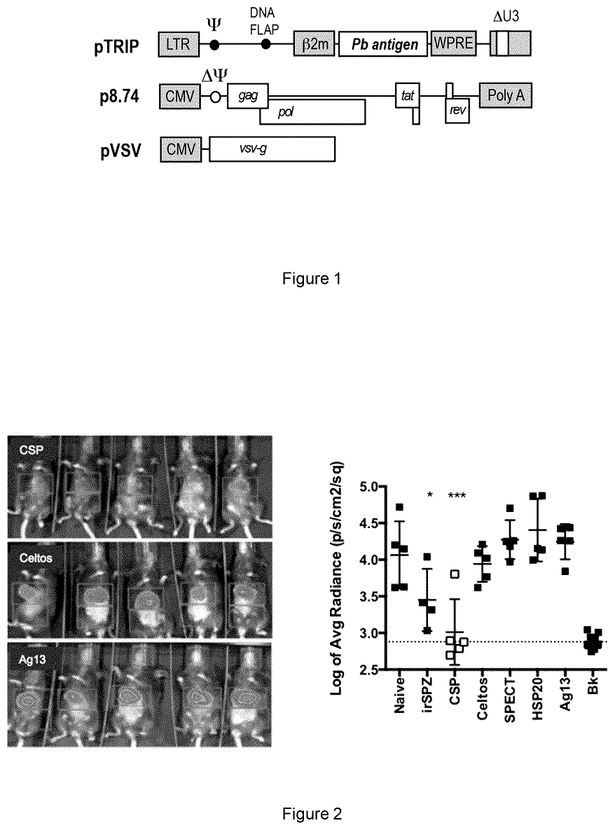

In a proof-of-concept experiment aimed at validating the viability of the strategy to screen antigens at a medium-throughput, the inventors ordered mouse-codon optimized synthetic genes of Pb CSP (SEQ ID No. 11), a known protective antigen, and of more 4 other sporozoite antigens (Celtos, SPECT, HSP20 and Ag13), which were previously correlated with protection¹³. The synthetic plasmodial genes were cloned in to the pTRIP vector plasmid, which drives their expression in mammalian cells via the immediate-early cytomegalovirus promoter (CMV) and the post-transcriptional regulatory element of woodchuck hepatitis virus (WPRE) (FIG. 1, SEQ ID No. 1). These two elements assure a strong expression of the antigen in a wide variety of mouse cells in vivo. HIV-1 derived lentiviral particles were produced by transient co-transfection of HEK 293T cells with three helper plasmids encoding separate packaging functions, the pTRIP vector plasmid containing the synthetic plasmodial gene, the envelope expression plasmid encoding the glycoprotein G from the Vesicular Stomatitis Virus, Indiana (VSV^{IND}) or New Jersey (VSV^{NJ}) serotypes, and the p8.74 encapsidation plasmid (FIG. 1). This co-transfection generates integrative but replication-incompetent pseudotyped lentiviral particles capable of transducing dividing and non-dividing cells-including dendritic cells—and inducing potent cellular⁶ and humoral⁷ memory responses. The particles were collected 48 hours after co-transfection and each batch of vector were titrated in Hela cells by quantitative PCR. This functional titration assay gives the concentration of particles capable to transfer one copy of the gene per cell and will be expressed in Transducing Units (TU)/mL. Plasmid sequences are shown in the figures and their sequences are provided in the sequence listing.

Groups of five mice were immunized with a single intra-muscular dose of 5e7 TU of ultracentrifugation-concentrated VSV^{IND} pseudotyped lentiviral particles (LPs). Thirty days after immunization, mice were challenged with 10,000 bioluminescent sporozoites inoculated sub-cutaneously in the footpad. Two days later, the parasite load in the liver was measured by bioluminescence. Surprisingly, CSP-immunization decreased 15×-fold the parasite load in the liver after a challenge using 10,000 bioluminescent sporozoites, versus a 5×-fold decrease in animals immunized intravenously with 50,000 irradiated sporozoites, our golden standard of protection (FIG. 2). This preliminary and promising result validated the high performance of the present method to functionally identify new protective antigens and showed the feasibility to scale-up our test samples.

The inventors next aimed at the transposition of these optimal experimental conditions to those of a larger screening. This transposition included the validation of the use of non-concentrated LPs, the choice of the best promoter driving the expression of the plasmodial antigens, and the dose of immunization.

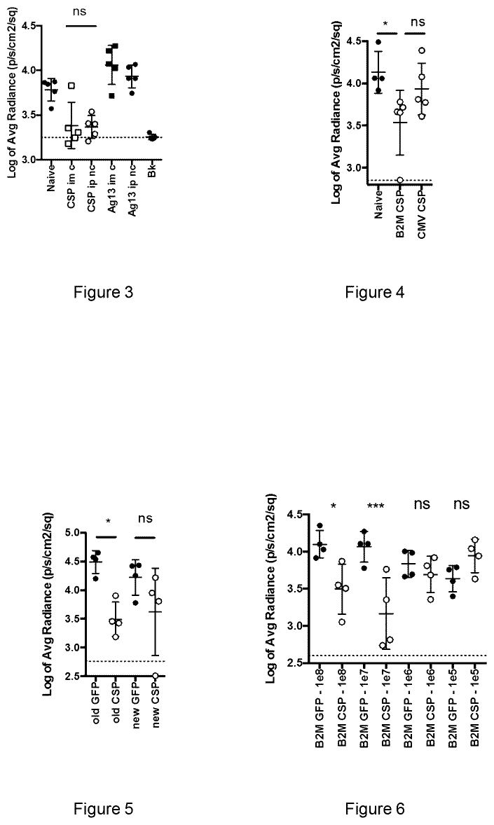

The first parameter tested was the use of non-concentrated, instead of concentrated LPs, to avoid a costly and time consuming ultracentrifugation concentration step in the protocol of LP production, which requires large volumes of non-concentrated LP suspensions. FIG. 3 shows that there is no significant difference between protection induced by the same dose (5×10⁷ TU) of concentrated LPs injected intramuscularly (CS im c, 50 uL) and non-concentrated LPs injected intraperitoneally (CS ip nc, 700 μL). Protection was measured by reduction in the liver infection, as assessed by bioluminescence after a challenge of 5,000 sporozoites injected subcutaneously 30 days following immunization. As negative control of protection the inventors used mice immunized with Pb Ag13, determined previously as a non-protective antigen (FIG. 1).

Next two promoters were tested to identify which one induced the best protection using the codon optimized Pb CSP. The inventors compared the use of the strong and constitutive cytomegalovirus (CMV) promoter versus a human beta-2 microglobulin (B2M) promoter, which direct gene expression in many cell types, particularly in dendritic cells. FIG. 4 shows that CSP-induced protection was slightly better, although not statistically significant, using the B2M promoter at an immunization dose of 1×10⁷ TU of non-concentrated LP. Therefore the inventors further adopted this promoter in our constructs.

During this period of optimization the inventors observed some variations in the CSP-induced protection using the same stock of LPs, as can be seen in the FIG. 4. The inventors asked if this variability could be due to the process of mouse acclimation, including the modification of mouse microbiota. To test this hypothesis a group of mice purchased from Elevage Janvier (4 weeks-old) was reared in the animal facility for 3 weeks before immunization (group old). A second group of mice (7 weeks-old) was purchased and put in cages 3 days before the immunization (group new). Both groups were intraperitoneally immunized with 1×10⁷ TU of non-concentrated LPs. As shown in the FIG. 5, mouse acclimation of 3 weeks resulted in a significant and more homogeneous protection when compared to 3 days of acclimation. Consequently, the inventors adopted this period of acclimation in all our subsequent experiments.

Next, the best protective immunization dose was tested, ranging from 1×10⁸ to 1×10⁵ TU of B2M CSP non-concentrated LPs. As shown in FIG. 6, significant protection was observed using 10⁷ and 10⁸ TU, and the best protective activity was observed using an immunization dose of 1×10⁷ TU. In this experiment the inventors also observed a gradual loss of SPZ infectivity over time, as evidenced in the GFP groups, due to the use of a single SPZ stock to challenge all animals. To reduce the multiple shocks of temperature due to the manipulation of the stock tube, kept on ice between injections, the inventors prepared a SPZ stock for each group in the subsequent challenges and this variation disappeared.

In summary, an immunization protocol was set up based on CSP that relied on a single intraperitoneal injection of 10⁷ TU of non-concentrated VSV^{IND} B2M LP in C57BL/6 mice of 7 weeks-old, acclimated for 3 weeks in the animal facility. In the pooled data, this protocol leaded in average to a ˜5-fold decrease in the parasite liver load, as assessed by bioluminescence imaging, using a subcutaneous challenge of 5,000 luciferase-expressing SPZ.

However, this bioluminescent method of detection of parasites presents some disadvantages such as the use and associated costs of anesthesia and luminescent substrate, limited capacity of analysis of a few animals per acquisition, being time-consuming and not sensible enough to predict sterile protection. Therefore, the inventors decided to use fluorescent parasites to check protection by measuring parasitemia at day 4, 5, 6 and 10 post-inoculation by flow cytometry. The inventors analyze at least 100,000 red blood cells, which gives the sensibility to detect a parasitemia of 0.001%. At day 4 to 6, parasites grow exponentially in the blood therefore the log transform of parasitemia can be fitted using a linear regression where the slope represents the time of parasite replication per day. Consequently, the inventors use this parameter to determine if the immunization impacts the parasite growth in the blood. For quantifying protection, the inventors used the log of parasitemia at day 5 post inoculation. This represents an indirect measure of liver infection and it is more robust than the measure at day 4 because more events of infected blood cells are registered. Finally the inventors defined that immunized mice are sterile protected if infected red blood cells are not detected after 10 days post inoculation. After defining the protocol of immunization and the method for the quantification of parasite infection the inventors started to screen the protective activity of down-selected antigens.

### 2. Parameterized Selection of Antigens

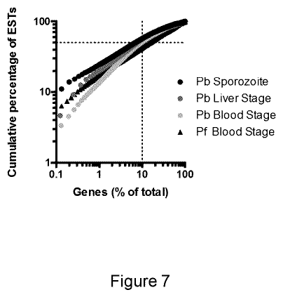

By merging proteomic and transcriptomic data using PlasmoDB (www.plasmodb.org), the inventors identified ˜9000 genes expressed in plasmodial pre-erythrocytic stages—salivary gland sporozoites and liver-stages—of three different plasmodial species, with 3654 syntenic orthologs in Plasmodium falciparum (Pf), the most lethal human-infecting plasmodial species. By analyzing the repertoire of pathogen transcripts, as inferred by the amount of expressed sequence tags (ESTs) obtained in cDNA libraries of different stages and species of malaria parasites, the inventors have observed that ˜50% of the total amount of ESTs are coming from only ˜10% of genes represented in these libraries, corresponding to approximately 100 genes in these libraries (FIG. 7). Therefore, by focusing on the ˜100 most abundant transcribed genes the inventors could target about 50% of the putative (to be translated) antigenic mass of a given parasite stage. Accordingly, the inventors selected ˜50 abundantly transcribed genes coding for conserved proteins with high probability of being expressed/presented on the surface of the parasite/infected cell, giving priority to candidates containing T cell epitopes predicted by IEDB MHC binding algorithm (http://tools.iedb.org/mhci/). A Kozak consensus sequence, a translational start site, was added to these down-selected genes, which were then mammalian codon-optimized and synthesized by MWG Eurofins (listed in the figures). These synthetic codon-optimized down-selected plasmodial genes were then cloned into the B2M pTRIP plasmid and produced as non-concentrated VSV^{IND} LPs.

### 3. First Screening of Protective Antigens Using a Single Dose of LPs

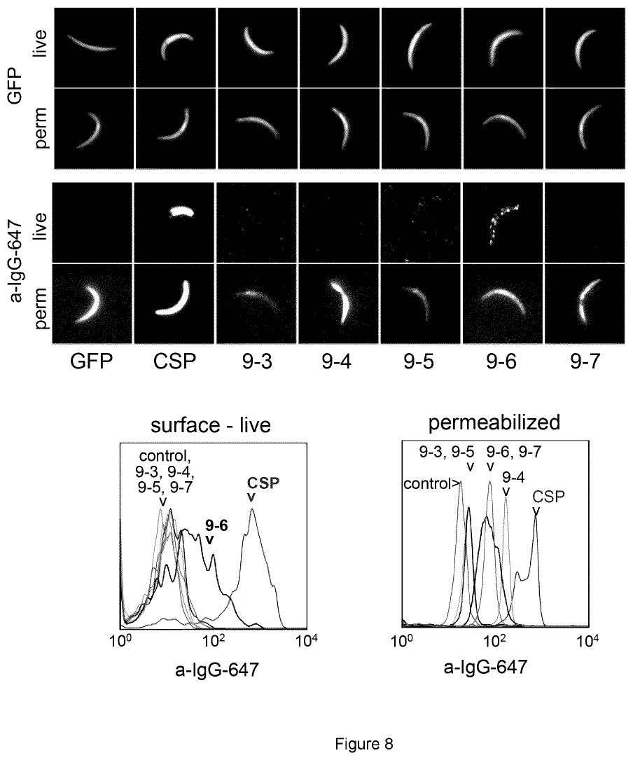

Usually, 6-10 plasmodial antigens were tested by experiment, with a negative (GFP) and positive (CSP) control of protection. After three weeks post-immunization, the immune-sera were tested on permeabilized and non-permeabilized sporozoites, allowing the determination of (i) the efficiency of the host humoral response and therefore the immunogenicity of the lentivirus-delivered antigen, and (ii) the localization of the parasite antigen (surface vs intracellular). As shown in the FIG. 8, where the inventors immunized mice with putative GPI-anchored antigens, surface antigens were identified by flow cytometry and immunofluorescence (CSP and 9-6). The sera of GFP and CSP group served, respectively, as positive control for intracellular and surface antigen localization.

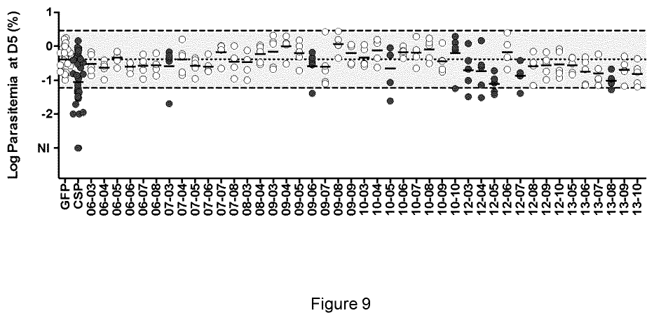

Four weeks post-immunization the animals were challenged with 5,000 GFP-expressing sporozoites, microinjected in the footpad of immunized mice. Parasitemia was determined by flow cytometry. To define protection, parasitemia of all GFP groups (day 5 post-infection, n=35) was log transformed, pooled and the 95% tolerance interval was calculated (FIG. 9). All animals below the inferior limit of the tolerance interval, which represents a ˜8-fold decrease in parasitemia compared to the mean log of parasitemia of the GFP group, were considered protected. As positive control, 43% of animals (15/35) were protected by CSP immunization with a mean decrease of ˜5 fold in comparison to the GFP group. 9% of them (3/35) became sterile protected after sporozoite challenge.

In the first set of 43 antigens tested (FIG. 9), we identified 9 PE antigens that protected at least one out of five immunized mice (black circles; 07-03, 09-06, 10-05, 10-10, 12-03, 12-04, 12-05, 12-07 and 13-08). Three of them were also identified as sporozoite surface antigens (09-06, 10-05 and 10-10).

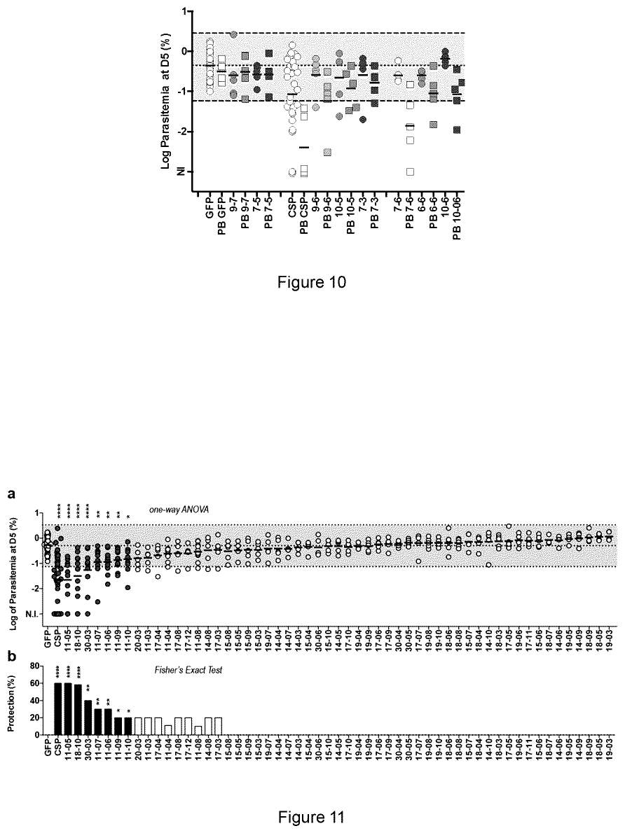

To verify the robustness of our screening, the inventors selected 4 protective antigens (CSP, 09-06, 10-05 and 07-03), 6 non-protective antigens (GFP, 09-07, 07-05, 07-06, 06-06 and 10-06), and instead of only one immunization dose, the inventors administered one dose of 5×10⁵ TU of non-concentrated VSV^{NJ} B2M LPs and one month later, a second dose of 1×10⁷ TU of VSV^{IND} B2M LPs. As shown in the FIG. 10, the inventors observed three patterns of infection profile when the inventors compared one (circles, data from FIG. 9) and two immunization doses (squares, PB). For the non-protective antigens GFP, 09-07 and 07-05, the second dose of LP did not change the profile of infection, as expected. For the protective antigens CSP (***P<0.001), 09-06, 10-05 and 07-03, the second dose of LP increased the number of protected mice and/or decreased the average parasitemia, also, as expected. Notably, the non-protective antigens 07-06, 06-06 and 10-06 as assessed by one dose of LP immunization, showed a strong protective activity, including a sterile protected mice (PB 7-6), when administered twice in mice.

These results validated some of our protective antigens detected with a single immunization dose, but also showed that some good protective antigens were not detected in our first screen, leading to the decision of repeating the screening using two immunization doses.

### 4. Second Screening of Protective Antigens Using Two Doses of LP

By functionally screening the protective activity of 55 down-selected plasmodial PE antigens using the protocol of two immunization doses, the inventors identified 16 antigens that protected at least one immunized mice per group. Among these 16 antigens, 7 of them (black circles/bars in the FIG. 11) conferred significant protection when compared to animals immunized with the GFP, both when analysing the number of protected mice (Fisher's Exact test) or the mean of the log parasitemia (ANOVA).

All of them presented a similar or an inferior protective activity when compared individually to our standard of protection, the CSP (FIG. 11). Five of them are molecules with assigned function (11-05, 11-06, 11-07, 30-03 and 18-10) and two are proteins with no predicted function (11-09 and 11-10). The structure of these Pb protective antigens is shown in the FIG. 12 and the alignment of these proteins with their respective orthologs from human-infecting parasites, P. falciparum (Pf) and P. vivax (Pv), and macaque-infecting parasite P. cynomolgi, is represented in the FIG. 13. As shown in table I, the percentage of identical amino acids between orthologs varied from 75 to 38% (Pb vs Pf), 78 to 33% (Pb vs Pv) and 79 to 26% (Pf vs Pv). The most conserved genes (>50% identity) are 30-03, 11-09, 11-10 and 11-06 orthologs. Antigens with divergent repetitive sequences are penalized in the alignment by insertional gaps, presenting less percentage of identity.

TABLE I
Percent Identity Matrix created by CLUSTAL 2.1.
Amino acid sequence of Pb antigen swere pBlasted
against Pf and Pv taxids (organism) and the best
matched sequence was used to align the orthologous proteins
using MUSCLE(http://www.ebi.ac.uk/Tool/msa/muscle).
The table shows the percentage of identical amino acids
between species. Raw data is presented in the figures

| | Amino acid identity (%) | | |
|---|---|---|---|
| Antigen | Pb/Pf | Pb/Pv | Pf/Pv |
| 30-03 | 74.65 | 77.90 | 73 |
| 11-09 | 66.19 | 66.19 | 79.05 |
| 11-06 | 64.92 | 63.40 | 64.44 |
| 11-10 | 56.94 | 50.15 | 62.28 |
| CSP | 42.06 | 33.53 | 26.36 |
| 18-10 | 39.60 | 41.23 | 49.30 |
| 11-05 | 38.75 | 44.67 | 42.86 |
| 11-07 | 37.53 | 33.42 | 46.30 |

In a decreasing order of protection the first antigen identified is TRAP¹⁴ (thrombospondin related anonymous protein; 11-05) (SEQ ID N° 20 and 21), which validated our method of screening since immunization with TRAP is known to induce protection both in rodents¹⁵ and humans¹⁶. TRAP is a type I transmembrane protein harbouring two extracellular adhesive domains, a von Willebrand factor type A domain and a thrombospondin type 1 domain, followed by a proline-rich repetitive region. TRAP is stored in micronemal secretory vesicles and following parasite activation, the protein is translocated to the surface of sporozoites where it serves as a linker between a solid substrate and the cytoplasmic motor of sporozoites. Intriguingly, anti-TRAP antibodies do not impair parasite motility and infectivity¹⁷ CD8+ T cells seem to mediate the protection mediated by TRAP immunization^{10, 15, 16, 18}.

The second protective antigen identified is an inhibitor of cysteine protease (ICP, 18-10)¹⁹ (SEQ ID N° 29, 30). ICP seems to be involved in the motility and infectivity capacity of sporozoites via the processing of CSP^{20, 21}, as well as, in the parasite intra-hepatic development²². Although the protein does not present structural signatures of membrane localization, there is evidence that the protein is located on the surface of sporozoites^{19,20}. Opposing results are published regarding the secretion of the protein following parasite activation^{21,22}. Similarly, there are contradictory results regarding the inhibition of host cell invasion by sporozoites in vitro in the presence of anti-ICP immune sera^{20,23}.

The third protective antigen identified is a metallopeptidase (Falcilysin/Bergheilysin, 30-03) 24 (SEQ ID N° 38 for Bergheilysin A, N° 47 for Bergheilysin B, and N°39 for the entire Bergheilysin ORF). This protease seems to be involved in the catabolism of hemoglobin in the parasite blood stages²⁵. A H-2Kb-restricted CD8 T cell epitope was recently described during the parasite blood infection²⁵ suggesting that CD8 T cells could mediate the protection elicited by the antigen 30-03 during the hepatic infection.

The fourth protective antigen is a GPI-anchored protein (P113, 11-07) (SEQ ID No. 58 and 59) initially described in blood stages¹⁶ and also expressed in PE stages. P113 seems to be important for liver infection, dispensable for blood infection, but its precise function is unclear¹⁷.

The fifth antigen is the pore-forming like protein SPECT2 (11-06)²⁸ (SEQ ID N° 49 and 50). This protein has a membrane attack complex/perforin (MACPF) domain and is involved in the sporozoite cell traversal activity, being important for the progression of sporozoites in the dermis²⁹ and survival to phagocytosis in the liver³⁰.

The sixth antigen identified is a hypothetical protein that the inventors called 11-09 or Ag40 (SEQ ID N° 67 and 68). This protein has 4-5 annotated transmembrane domains. Deletion of the gene coding for the antigen 11-09 caused impairment of Pb parasite development in the liver.

The seventh antigen is also a hypothetical protein that the inventors called 11-10 or Ag45 (SEQ ID N° 76 and 77). This protein doesn't have annotated domains, but possesses a central region with negatively charged amino acids. Recently the 11-10 ortholog of Plasmodium yoelii, another rodent-infecting plasmodial species, was also identified as a protective antigen²¹. The deletion of the gene coding for the antigen 11-10 blocked the Pb sporozoite invasion of salivary glands and completely abolished the capacity of sporozoites to infect the liver.

To determine if CSP based protection could be additively or synergistically improved by the combination of antigens, the inventors assessed the protection elicited by a sub-optimal dose of CSP (5×10⁵ TU of VSV^{NJ}/5×10⁶ TU of VSV^{IND} B2M LPs) in the absence or presence of a usual dose of protective antigens (5×10⁵ TU of VSV^{NJ}/1×10⁷ TU of VSV^{IND} B2M LPs). This protection induced by CSP+protective antigens was compared to the protection elicited by these antigens alone (data from FIG. 11). As negative control the inventors used animals immunized with the usual dose of GFP LPs. FIG. 14 shows that 4 antigens when combined with a sub-optimal dose of CSP (CSP+11-03, +11-10, +11-07 and +11-05, triangles) did not change the average of protection when compared to the protective activity elicited by these antigens administered alone. For two antigens, the antigen combination (CSP+11-09 and CSP+11-06) showed a tendency of better protection (˜10 fold), but not statistically significant.

### 5. Identification of Multi-Antigenic Formulations Capable of Sterilizing Sporozoite Infection Via a CD8+ T Cell Response

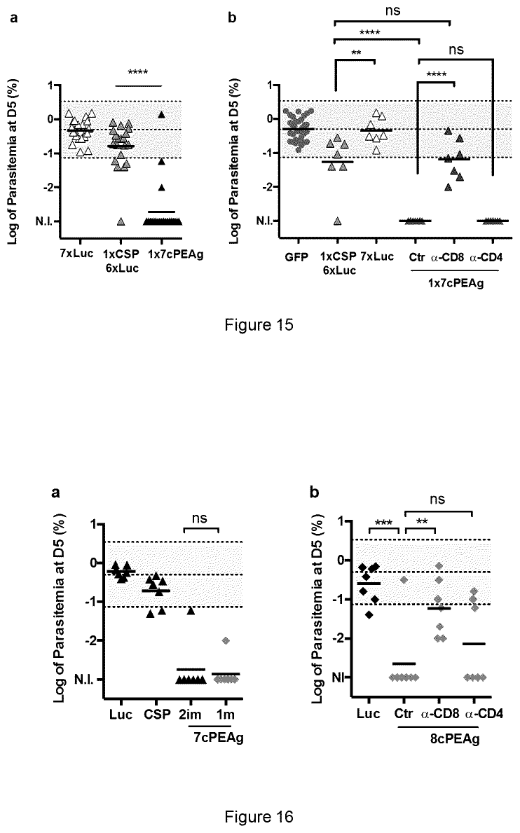

Since testing all possible combinations of antigens was technically unfeasible, the inventors decided to evaluate the protection elicited by the combination of these multiple protective antigens. Remarkably, two immunizations of mice with the combination of CSP and 6 of these antigens (11-05, 11-06, 11-07, 11-09, 11-10, 18-10) elicited sterile protection in the vast majority of challenged animals (7PEAg, 86-100%, FIG. 15). This percentage of sterile protection was far superior to the protection conferred by CSP in the same experimental conditions (0-14%). Depletion of CD8+ cells (a-CD8) just before the challenge, but not of CD4+ cells, decreased this protection to the level of that induced by CSP, suggesting that CD8+ T cells mediate the extra-protection elicited by the addition of these 6 PE antigens.

The same protective efficacy was observed using only a single immunization for the 7PEAg or for the 7PEAg+30-03 (FIGS. 16a and 16b, 8PEAg), as well as, the dependence on CD8+ T cells for the sterilizing immunity of 8PEAg (FIG. 16b). Since the antigen 30-03 is a large molecule and did not improve sterile protection when administered with the 7PEAg, the inventors excluded it from further analysis.

### 6. Design of a Chimeric Antigen Containing the Protective Domains of Down-Selected PE Antigens

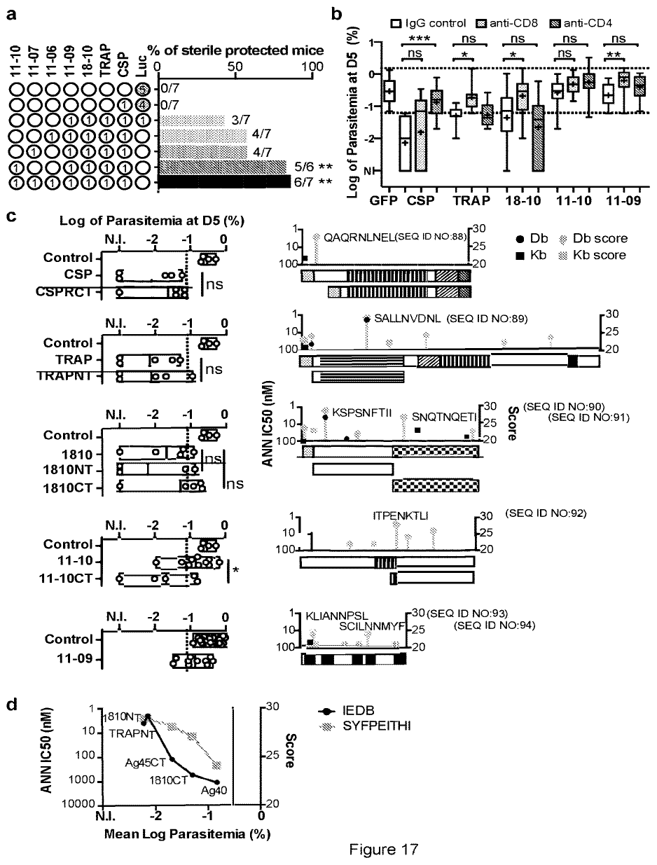

To determine a minimal antigenic composition capable of eliciting this additional protective CD8+ T cell response, the inventors first identified the antigens whose protective activity was dependent on these T cells. Protection induced by two immunizations using TRAP, 18-10 and 11-09 was significantly reduced after depletion of CD8+ cells, as shown in the FIG. 17b. Protection induced by two immunizations using 11-10 was reduced after depletion of CD8+ cells but it was not statistically significant (FIG. 17b). Therefore the inventors grouped CSP with the CD8+ dependent protective antigens, TRAP, 18-10 and 11-09 and added separately 11-10, 11-07 and 11-06 to identify a minimal antigenic combination capable of sterile protect immunized animals like the complete combination of antigens. As shown in FIG. 17a, the combination of 5 antigens, CSP+TRAP, 18-10, Ag40 and Ag45 induced comparable level of sterile protection elicited by the combination of the 7PEAg.

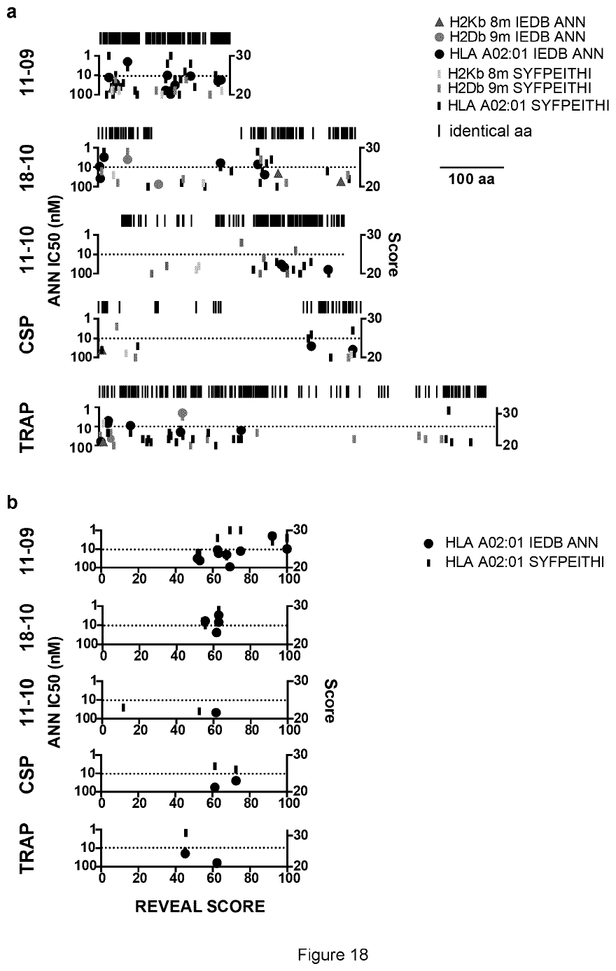

In order to combine the protective domains of each of these 5 antigens in a single chimeric molecule and thus avoid the costs associated with the production and delivery of five different antigens, the inventors mapped the protective regions of each antigen according to the localization of predicted epitopes binding to MHC class I molecules (FIGS. 17c and 18) and structural-functional conserved motifs (FIGS. 12, 13, 17c and 18). As shown in the FIG. 17c, all tested domains presented either a better (11-10 CT) or similar protective activity when compared to the entire antigen. The level of mean protection elicited by the domains of antigens inducing protective CD8+ T cells correlated with the score (P<0.01) or affinity (P=0.01) of CD8+ T cell epitopes respectively predicted by SYFPEITHI and IEDB (SEQ ID NO:89 to SEQ ID NO:94) (FIGS. 17c and 17d). Importantly, the mapping of protective domains allowed the reduction of ˜2000 basepairs in the final chimeric PE antigen construct. Due to its small size, Ag40 was not split in domains and the data presented in the FIG. 17c comes from the experiment showed in the FIG. 11.

Analysis of the distribution of epitopes of Pb antigens predicted to bind to MHC class I molecules of C57BL/6 mice (H2-Kb, H2-Db) or of the Pf orthologues predicted to bind to HLA A02:01, a high prevalent human HLA allele, revealed that most of predicted good binders are clustering in the regions that are conserved among different plasmodial species (Pb, Pc, Pv and Pf, FIG. 18). This renders possible the utilization of the Pb protective regions mapped in the FIG. 17 to select the correspondent regions in the Pf orthologues. In addition, the inventors validated the binding of the best predicted Pf epitopes to the HLA A02:01 class I molecule using the REVEAL® binding assay developed by Proimmune, which allows the quantification of the binding and stabilization of the complex formed by the tested peptide, HLA A02:01 and 2-microglobulin (FIG. 18).

In summary, using a parameterized selection of antigens, a screening based on lentiviral vaccination and a direct measurement of protection in vivo against a stringent sporozoite challenge, the inventors identified 8 protective antigens, including the vaccine candidates CSP and TRAP, out of 55 tested antigens. All these 8 antigens are conserved across several plasmodial species. Remarkably, immunization using a combination of seven or eight of these antigens elicited sterile protection in the vast majority of challenged mice, either using one or two immunizations. More importantly, this protection was far superior than the one elicited by CSP, so far the best protective PE antigen. Depletion of CD8+ T cells abolished sterilizing immunity, indicating that these cells are essential for this protective phenotype, similarly to the protection conferred by irradiated sporozoites. A minimal combination of 5 of these antigens was also capable of eliciting sterile protection in most of challenged animals. Mapping of the protective domains of these 5 antigens allowed the design of a chimeric antigen containing the fused protective domains of these 5 down-selected antigens. The human-infecting parasite orthologs of these protective antigens, or of their protective domains are potential candidates for being used in the development of a malaria vaccine formulation containing multiple protective antigens or multiple protective domains fused in a single molecule.

### Chimeric Antigenic Polypeptide as a Fusion of Protective Domains of Plasmodium Antigens and Immunogenic Response

Immunization using a combination of CSP and 6 or 7 of screened conserved protective antigens delivered by lentiviral particles, were shown to confer sterile protection in ≈85% of mice challenged with 5,000 sporozoites (FIG. 16). Extra-protection induced by the addition of these antigens to CSP was abolished after depletion of CD8+ T cells.

The inventors have also shown that combination of four pre-erythrocytic conserved protective antigens (4cPEAg i.e., 4 separated antigens used in combination for administration) administered together with CSP elicited sterile protection at a similar level of that of the combination using 7 or 8 antigens (FIG. 17). To further reduce the total size of antigens, the protective domains of these four antigens were selected based on amino acid conservation among plasmodial species and the presence of CD8+ T cell epitopes predicted to be good binders to class I MHC molecules (FIGS. 17 and 18). Their protective activity was then tested and compared to the protection elicited by the entire antigen. After identification of the protective domains (PD) of these 4cPEAg the inventors elaborated the construction of a chimeric antigen and selected a first construct formed by the fusion of 4 antigenic domains, i.e., the PD of the antigen 18-10 NT (N-terminal)+18-10 CT (C-terminal), with the PD 11-10 CT, followed by the PD TRAPNT and the antigen 11-09. This chimeric antigen was called Fusion 4cPEAg and it is structure is shown in the FIG. 24a. A particular amino acid sequence of this construct together with its DNA is provided as SEQ ID No. 116 and 115 for Plasmodium Berghei. When tested in a protocol of two immunization doses administered four weeks apart and in combination with CSP (5×10⁵ TU and 3×10/TU of LPs per antigen in the first and second immunization, respectively) Fusion 4cPEAg of P. berghei was as efficient as the combination of the 4cPEAg, sterilizing the infection of six out of seven challenged mice (FIG. 24b, Fusion 4cPEAg+CSP versus 4cPEAg+CSP). Notably, using this immunization dose, the 4cPEAg in the absence of CSP elicited the same level of sterile protection obtained with the combination of the 7cPEAg (FIG. 15). Single immunization with CSP (1×10⁷ TU) or the Fusion 4cPEAg (4×10⁷ TU), despite decreasing in almost ten fold the average parasitemia, only minimally sterilely protected the challenged mice (1 out of 7, FIG. 25, CSP and Fusion 4cPEAg groups). On the other hand, when combined, Fusion 4cPEAg+CSP sterilely protected 5 out of the 7 challenged mice (FIG. 25, Fusion 4PEAg+CSP group), achieving similar level of sterile protection of combinations using 5, 7 and 8cPEAgs (FIGS. 15-17).

This data confirmed the protective efficacy of the chimeric antigen prepared as a fusion of the protective domains, which satisfactorily substituted the 4cPEAg.

Since predicted CD8+ T cell epitopes clustered in conserved regions of the antigens, independently of the plasmodial species and MHC class I restriction (FIG. 18), this particularity was used to select the regions of P. falciparum 4cPEAg, corresponding to the protective domains of P. berghei 4cPEAg. To strengthen the analysis showed in the FIG. 18, more HLA class I and II alleles were analyzed, including the mapping of 9-mers peptides predicted to bind to HLA-DRB1*01:01, *03:01, *04:01, *04:05, *07:01, *08:02, *09:01, *11:01, *12:01, *13:02 and *15:01, to the HLA A*01:01, *02:01, *02:03, *02:06, *03:01, *11:01, *23:01,*24:02, *26:01, *30:01, *30:02, *31:01, *32:01, *33:01, *68:01 and *68:02, and to the HLA-B*07:02, *08:01, *15:01, *35:01, *40:01, *44:02, *44:03, *51:01, *53:01, *57:01 and *58:01 (FIG. 26). This extended analysis corroborated the initial observation, using the HLA A*02:01 and the H2 Db/Kb (FIG. 18), that good binders tend to cluster in regions associated with structural/functional conserved domains, transmembrane domains, as well as in signal peptide and GPI-anchoring sequences. Based on this clustering of epitopes, the sequences/structures of P. berghei antigens were used to retrieve the cognate regions in P. falciparum antigens as shown in the FIG. 26. These putative protective domains were fused as in P. berghei avoiding the creation of neo-epitopes in the junction of antigens/protective domains as shown in the FIG. 26e. When inevitable, an amino acid residue was introduced in the fusion sequence to avoid the creation of neo-epitopes with high binding affinity to HLA. The only amino acid added in the Pf fusion 4cPE Ag was a glutamic acid (E) at the end of the Pf11-10 CT. the amino acid sequence of the obtained construct and its DNA is provided as SEQ ID No. 118 and 117

A further chimeric construct was designed in order to benefit from the immune response elicited by the CSP protein, taking into consideration that the lentiviral particles were shown to sustain the presence of large antigens. accordingly a fusion of 5 antigens or their protective domains was prepared, adding the CSP antigen to the fusion 4cPE Ag in a structure containing the CSP devoid, or not, of its signal peptide and devoid of its GPI followed by the PD 18-10 NT and CT, the PD 11-10 CT, followed by the PD TRAPNT and the antigen 11-09. The construct obtained for P. berghei has the amino acid sequence of SEQ ID No. 120 and the DNA of SEQ ID No. 121 or has the amino acid sequence of SEQ ID No. 122 and the DNA of SEQ ID No. 123. Specific amino acid residues were deleted from the original antigens where appropriate in order to preclude the formation of neo-epitopes.

### Material and Methods

Parasite strains: Plasmodium berghei ANKA strain constitutively expressing the GFP under the control of the hsp70 promoter (Ishino et al, 2006) was used in the challenges using parasitemia, quantified by flow cytometry, as protective readout. Plasmodium berghei ANKA strain constitutively expressing a GFP-luciferase fusion under the control of the eef-1alfa promoter (Franke-Fayard et al, 2008) was used in the challenges using liver infection, assessed by bioluminescence, as protective readout. Of note, parasitemia quantified using hsp70-gfp parasites was at least 10 times more sensible than using eef-1a gfp:luc parasites due to more intense expression level of GFP.
Ishino T, Orito Y, Chinzei Y, Yuda M (2006) A calcium-dependent protein kinase regulates Plasmodium ookinete access to the midgut epithelial cell. Mol Microbiol 59:1175-1184.
Franke-Fayard B, Djokovic D, Dooren M W, Ramesar J, Waters A P, et al. (2008) Simple and sensitive antimalarial drug screening in vitro and in vivo using transgenic luciferase expressing Plasmodium berghei parasites. Int J Parasito/38:1651-1662.

Mouse strains: C57BL/6 Rj and Swiss mice were purchased from Elevage Janvier (France). All experiments were approved by the Animal Care and Use Committee of Institut Pasteur (CETEA 2013-0093) and were performed in accordance with European guidelines and regulations (MESR-01324).

Production of Lentiviral Particles stock: Down-selected plasmodial antigens were synthesized by Eurofins MWG as mouse codon-optimized genes with the addition of a Kozak consensus sequence (GCCACCATGGCT(C) (SEQ ID No. 85 and 86), representing the first 12 nucleotides in the coding sequences of the antigenic polypeptides), encompassing the first translated ATG. This modification adds an extra alanine after the first methionine. A BamHI (GGATCC-SEQ ID No. 87) and Xho I (CTCGAG-SEQ ID No. 88) restriction sites were also inserted in the 5′ and 3′ extremities of the construct, respectively. These synthetic codon-optimized genes were then cloned into the BamHI and Xho I restriction sites of the pTRIP plasmid harboring either the CMV or B2M promoter (FIGS. 16 and 17). Lentiviral particles were produced by transient calcium co-transfection of HEK 293T cells with three helper plasmids encoding separate packaging functions, the pTRIP vector plasmid containing the synthetic plasmodial gene, the envelope expression plasmid encoding the glycoprotein G from VSV (Vesicular Stomatitis Virus, Indiana (FIG. 19) or New Jersey (FIG. 20) serotypes) and the p8.74 encapsidation plasmid (FIG. 18), containing the structural, accessory and regulatory genes of HIV. This co-transfection will generate integrative but replication-incompetent pseudotyped lentiviral particles. At 24 hours post-transfection, the cell culture medium was replaced by serum-free DMEM. Supernatants were collected at 48 hours post-transfection, clarified by low-speed centrifugation, and stored at −80° C. The lentiviral vector stocks were titrated by real-time PCR on cell lysates from transduced HEK 293T cells and titer were expressed as transcription unit (TU) per ml.

Immunization protocol: For the screening using one single dose of LPs, 4 weeks-old C57BL/6 mice (n=5 per group per experiment) were acclimated for 3 weeks and intraperitoneally immunized with a single dose of 1×10⁷ TU of non-concentrated VSV^{IND} B2M LPs. For the protocol using two immunization doses. 4 weeks-old C57BL/6 mice (n=5 per group per experiment) were acclimated for 3 weeks and intraperitoneally immunized with a first dose of 5×10⁵ TU of non-concentrated VSV^{NJ} B2M LPs. Thirty days after the first immunization, the animals received a second dose of 1×10⁷ TU of non-concentrated VSV^{IND} B2M LPs. For testing combinations of a sub-optimal dose of CSP+an optimal dose of down-selected antigens, mice were immunized twice, four weeks apart, with a sub-optimal dose of CSP (5×10⁵ TU of non-concentrated VSV^{NJ} B2M LP in the first immunization and 5×10⁶ TU of non-concentrated VSV^{IND} B2M LP in the second immunization) and the usual dose of protective plasmodial antigens (5×10⁵ TU of non-concentrated VSV^{NJ} B2M LP in the first immunization and 1×10⁷ TU of non-concentrated VSV^{IND} B2M LP in the second immunization). For testing the combination of multiple antigens, mice were immunized twice, four weeks apart, with 7× the individual dose (1 dose=5×10⁵ TU of non-concentrated VSV^{NJ} B2M LPs in the first immunization/1×10⁷ TU of non-concentrated VSV^{IND} B2M LPs in the second immunization) of the control antigen Al11-luciferase (Luc), with the individual dose of CSP plus 6 doses of Luc or with the individual doses of CSP and of the 6 conserved PE antigens (11-05, 11-06, 11-07, 11-09, 11-10 and 18-10). For this experiment the inventors used ultrafiltration and lenti-X (Clontech) concentrated stocks. The average volume of injection was 500 μL of LPs diluted in DMEM.

In all cases, thirty days after last immunization, mice were challenged with 5,000 GFP-expressing sporozoites micro-injected subcutaneously in the mice footpad.

Sporozoite challenge: Anopheles stephensi (Sda500 strain) mosquitoes were reared using standard procedures. 3-5 days after emergence, mosquitoes were fed on infected Swiss mice with a parasitemia superior to 2%, and kept as described in Amino et al, 2007. Between 20 and 23 days post-feeding, the salivary glands of infected mosquitoes were dissected in PBS, collected in 20 uL of sterile PBS on ice and disrupted using an eppendorf pestle. The suspension of parasites was filtered through a nylon mesh of 40 um, counted using Kova glasstic slide (Hycor) and adjusted to a concentration of 5,000 or 10,000 sporozoites/uL with sterile PBS. This suspension was divided in individual tubes, one for each group of immunized mice (n=4-7 per group), and kept on ice until the challenge. One microliter of parasite suspension was injected in the right footpad of mice using a Nanofil syringe (World Precision Instruments) with a 35 GA bevelled needle (NF35BV).

Amino R, Thiberge S, Blazquez S, Baldacci P, Renaud O, et al. (2007) Imaging malaria sporozoites in the dermis of the mammalian host. Nat Protoc 2:1705-1712.

Measurement of Parasite Infection: Hepatic parasite loads were quantified at ˜44 h by bioluminescence in fur shaved mice infected with GFP LUC parasites. Infected mice were first anesthetized with isoflurane and injected subcutaneously with D-luciferin (150 mg/kg, Caliper LifeSciences). After a 5 minutes incubation allowing the distribution of the substrate in the body of the anesthetized animals, mice were transferred to the stage of an intensified charge-coupled device photon-counting video camera box where anesthesia was maintained with 2.5% isoflurane delivered via nose cones. After 5 minutes of signal acquisition controlled by the Living Image software (Xenogen Corporation), animals were returned to their cage. Automated detection of bioluminescence signals by the system resulted in the generation of bioluminescence signal maps superimposed to the gray-scale photograph of the experimental mice. These images were then quantified using the Living Image software. Briefly, regions of interest (ROI) encompassing the liver were manually defined, applied to all animals and the average radiance within these ROIs was automatically calculated. Background signal was measured in the lower region of the abdomen, and the average values of background signal obtained.

Alternatively, blood infection was assessed by flow cytometry using hsp70-GFP parasites. At day 4, 5, 6 and >10 post-challenge, a millimetric excision was performed in the tail of mice allowing the collection of a drop of blood that was readily diluted in 500 μL of PBS. This diluted blood was analyzed using a flow cytometer. 500,000 events were analyzed at day 4 post-challenge and 100,000 events in the subsequent days. Non-infected mice after day 10 were considered as sterile protected.

Statistical analysis: Parasitemia data from GFP immunized control were log transformed and pooled for the calculation of 95% tolerance of interval with 95% of certitude. For the immunization protocol of one dose this limit comprised the interval of the mean value±2.49 SD (mean=−0.3906, SD=0.3392, n=35). Similarly, for the immunization protocol of two doses this limit comprised the interval of the mean value ±2.51 (mean=−0.3002, SD=0.3305, n=33). All mice with a log parasitemia inferior to the lower limit (mean-2.5 SD) were considered as significantly different from the control mice (P<0.05), and therefore considered as protected. In the protocol using two immunization doses, the difference in the numbers of protected mice, following the definition above, between the test group and the GFP control group was assessed using the Fisher's exact test. The average of the log parasitemia of the groups with significant differences in the Fisher's Test were compared to the GFP group using one-way ANOVA (Holm-Sidak's multiple comparison test).

### REFERENCES

1. http://www.who.int/mediacentre/factsheets/fs094/en/
2. RTS, S Clinical Trials Partnership. Lancet. 2015; 4; 386(9988):31-45.
3. Moorthy V S, Ballou W R. Malar J. 2009; 8:312.
4. http://www.who.int/immunization/topics/malaria/vaccine_roadmap/en/5. Seder R A, et al. Science. 2013; 341:1359-65.
6. Amino R, Ménard R. Nature. 2012; 484(7395):S22-3
7. Kester K E, et al. 2014. pii: S0264-410X(14)00822-6.
8. Mishra S, et al. Vaccine. 2011; 29(43):7335-42.
9. Murphy S C, Kas A, Stone B C, Bevan M J. Proc Natl Acad Sci USA. 2013; 110(15):6055-60.
10. Hafalla J C, et al. PLoS Pathog. 2013; 9(5):e1003303.
11. Iglesias, M. C., et al. J Gene Med. 2006; 8, 265-274.
12. Firat, H., et al. J Gene Med. 2002; 4, 38-45.
13. Doolan D L, et al. Proc Natl Acad Sci USA. 2003; 100:9952-7.
14. Robson K J, et al. Nature. 1988; 335(6185):79-82.
15. Khusmith S, et al. Science. 1991; 252(5006):715-8.
16. Ewer K J, et al. Nat Commun. 2013; 4:2836.
17. Gantt S, et al. Infect Immun. 2000; 68(6):3667-73.
18. Khusmith S, Sedegah M, Hoffman S L. Infect Immun. 1994; 62(7):2979-83.
19. LaCrue A N, et al. Mol Biochem Parasitol. 2006; 148(2):199-209.
20. Rennenberg A et al. PLoS Pathog. 2010; 6(3):e1000825.
21. Boysen K E, Matuschewski K. MBio. 2013; 4(6):e00874-13.
22. Lehmann C, et al. PLoS Pathog. 2014; 10(8):e1004336.
23. Pei Y, et al. Cell Microbiol. 2013 September; 15(9):1508-26.
24. Eggleson K K, Duffin K L, Goldberg D E. J Biol Chem.; 274(45):32411-7.
25. Poh C M, Howland S W, Grotenbreg G M, Rénia L. Infect Immun. 2014; 82(11):4854-64.
26. Gilson P R, et al. Mol Cell Proteomics. 2006; 5(7):1286-99.
27. Offeddu V, Rauch M, Silvie O, Matuschewski K. Mol Biochem Parasitol. 2014; 193(2):101-9.
28. Ishino T, Chinzei Y, Yuda M. Cell Microbiol. 2005; 7(2):199-208.
29. Amino R, et al. Cell Host Microbe. 2008 Feb. 14; 3(2):88-96.
30. Tavares J, et al. J Exp Med. 2013; 210(5):905-15.
31. Speake C, et al. PLoS One. 2016; 11(7):e0159449.

We claim:

1. A chimeric antigenic polypeptide comprising a fusion of at least 2 antigens of a Plasmodium parasite infecting human or antigenic fragments thereof;
wherein said antigens are selected from the group consisting of TRAP, 18-10 (ICP), 11-10 (Ag45) and 11-09 (Ag40); and
wherein the antigenic fragments comprise T cell epitopes.

2. The chimeric antigenic polypeptide according to claim 1, comprising a fusion of at least 3 antigens of a Plasmodium parasite infecting human or antigenic fragments thereof, wherein at least 2 antigens are selected from the group consisting of TRAP, 18-10 (ICP), 11-10 (Ag45), and 11-09 (Ag40), and one antigen is CSP.

3. The chimeric antigenic polypeptide according to claim 2, whose primary structure results from a fusion of the following antigens and/or fragments thereof:
i. the TRAP antigen, the 18-10 antigen devoid of its signal peptide, the C-terminal fragment of the 11-10 antigen, and antigen 11-09 (Ag40) in any order of fusion; or
ii. the CSP antigen, the TRAP antigen, the 18-10 antigen devoid of its signal peptide, the C-terminal fragment of the 11-10 antigen, and antigen 11-09 (Ag40) in any order of fusion.

4. The chimeric antigenic polypeptide according to claim 1, wherein the amino acid sequence of the antigens of Plasmodium parasite and/or antigenic fragments consists of:
a. the amino acid sequence of SEQ ID No. 96 or SEQ ID No. 98 or SEQ ID No. 100, or SEQ ID No. 114 or the fusion of the amino acid sequences SEQ ID No. 96 and 98 for the 18-10 antigenic fragment fused to,
b. the amino acid sequence of SEQ ID No. 102 or SEQ ID No. 104 for the 11-10 antigenic fragment fused to,
c. the amino acid sequence of SEQ ID No. 106 or SEQ ID No. 108 for the TRAP antigenic fragment fused to,
d. the amino acid sequence of SEQ ID No. 110 or SEQ ID No. 112 for the 11-09 antigen.

5. The chimeric antigenic polypeptide according to claim 1, wherein the amino acid sequence:
i. comprises 1 to 3 additional junctional amino acid residues at the junction of SEQ ID No. 104 for the 11-10 antigenic fragment and SEQ ID No. 108 for the TRAP antigen, in particular contains one amino acid residue such as the Glutamic acid residue at said junction; or
ii. comprises a deletion of 1 to 6 or 1 to 5 amino acid residues present in the N-terminal end of the original antigen or antigenic fragment thereof present in the fusion such as the deletion of MA contiguous amino acid residues or MANG contiguous amino acid residues.

6. The chimeric antigenic polypeptide according to claim 1, wherein all the antigens or antigenic fragments thereof are from Plasmodium falciparum or from Plasmodium vivax.

7. The chimeric antigenic polypeptide according to claim 1, whose amino acid sequence is SEQ ID No. 116 or SEQ ID No. 120, or SEQ ID No. 122 or SEQ ID No. 118.

8. A combination or a composition of viral vectors comprising a vector that expresses the chimeric antigenic polypeptide as defined in claim 1, and a distinct vector that expresses the CSP antigen.

9. A combination or a composition comprising a human HIV-1 lentiviral vector expressing the chimeric antigenic polypeptide as defined in claim 1.

10. The combination or composition of claim 9, wherein the human HIV-1 lentiviral vector is a replication-incompetent pseudotyped HIV-1 lentiviral vector, wherein said vector contains a genome comprising a mammal codon-optimized synthetic nucleic acid wherein said synthetic nucleic acid encodes the antigen(s) and/or the chimeric antigenic polypeptide(s) of a Plasmodium parasite infecting a mammal.

11. The combination or composition of claim 9, wherein the genome of the lentiviral vector genome is obtained from a pTRIP vector plasmid wherein a Plasmodium synthetic nucleic acid encoding the antigenic polypeptide or polypeptidic derivative thereof has been cloned under control of a promoter functional in mammalian cells.

12. A formulation suitable for administration to a mammalian host comprising a combination of compounds or a composition according to claim 8 and an excipient suitable for administration to a host.

13. A method of immunizing a host against a malaria parasite infection, or against a parasite-induced condition or disease, comprising administering a formulation according to claim 12 to the host.

14. The method of claim 13, wherein the host is a human.


SEQUENCE LISTING (124 sequences)

SEQ ID NO: 1 - DNA, 4536 bp, synthetic construct
  misc_feature 1..4536: note = pTRIP CMV GFP
  source 1..4536: mol_type = other DNA
tggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcgatcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagctcgagctcaagcttcgaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattctgcagtcgacggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 4468 bp, synthetic construct
  misc_feature 1..4468: note = pTRIP B2M GFP
  source 1..4468: mol_type = other DNA
tggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaacctgtgagcctgcatgggatggatgacccggagagagaagtgttagagtggaggtttgacagccgcctagcatttcatcacggtggcccgagagctgcatccggagtacttcaagaactgctgatatcgagcttgctacaagggactttccgctgggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagctcgagctcaagcttcgaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattctgcagtcgacggtacctttaagaccaatgacttacaaggcagctgtagatcttagccactttttaaaagaaaaggggggactggaagggctaattcactcccaacgaagacaagatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 3 - DNA, 11904 bp, synthetic construct
  misc_feature 1..11904: note = PACKAGING 8.74 PLASMID
  source 1..11904: mol_type = other DNA
gggctgcaggaattaattcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgcgttgacgcgcacggcaagaggcgaggggcggcgactggtgagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagctcgagtgaccttcagaccttggcactggaggtggcccggcagaagcgcggcatcgtggatcagtgctgcaccagcatctgctctctctaccaactggagaactactgcaactaggcccaccactaccctgtccacccctctgcaatgaataaaacctttgaaagagcactacaagttgtgtgtacatgcgtgcatgtgcatatgtggtgcggggggaacatgagtggggctggctggagtggcgatgataagctgtcaaacatgagaattaattcttgaagacgaaagggcctcgtgatacgcctatttttataggttaatgtcatgataataatggtttcttagtctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgtt

SEQ ID NO: 4 - DNA, 5140 bp, synthetic construct
  misc_feature 1..5140: note = pCMV-VSV-INDco
  source 1..5140: mol_type = other DNA
ctggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatcctgatcagccaccatgaaatgcctgctctatctggccttcctgttcatcggcgtgaactgcaagttcaccatcgtgttcccccacaaccagaagggcaactggaagaacgtgcccagcaactaccactactgccccagcagcagcgacctgaactggcacaacgacctgatcggcaccgccctgcaggtgaagatgcccaagagccacaaggccattcaggctgatggctggatgtgtcatgccagcaagtgggtgaccacctgcgacttccggtggtacggccccaagtacatcacccacagcatccgcagcttcacccccagcgtggagcagtgcaaagagagcatcgagcagaccaagcagggcacctggctgaaccccggcttccccccccagtcctgcggctacgccaccgtgaccgacgccgaggccgtgatcgtgcaggtgaccccccaccacgtgctggtcgacgagtacaccggcgagtgggtggacagccagttcatcaacggcaagtgcagcaactacatctgccctaccgtgcacaacagcaccacctggcacagcgactacaaggtgaaaggcctgtgcgacagcaacctgatcagcatggacatcacctttttcagcgaggacggcgagctgtccagcctgggcaaagagggcaccggcttcagaagcaactacttcgcctacgagacaggcggcaaggcctgcaagatgcagtactgcaagcactggggcgtgcggctgcctagcggcgtgtggttcgagatggccgacaaggacctgttcgccgctgcccggttccctgagtgccccgagggcagcagcatcagcgcccccagccagaccagcgtggacgtgagcctgatccaggacgtggagagaatcctggactacagcctgtgccaggaaacctggtccaagatcagagccggcctgcccatcagccccgtggacctgagctacctggcccccaagaaccccggcaccggcccagccttcaccatcatcaatggcaccctgaagtacttcgagacacggtacatcagagtggacattgccgcccctatcctgagccggatggtgggcatgatcagcggcaccaccaccgagcgggagctgtgggacgactgggccccctacgaggatgtggagatcggccccaacggcgtgctgcggaccagcagcggctacaagttccccctgtacatgatcggccacggcatgctggacagcgacctgcacctgagcagcaaggcccaggtgttcgagcacccccacatccaggacgccgccagccagctgcccgacgacgagagcctgttcttcggcgacaccggcctgagcaagaaccccatcgaactggtggagggctggttcagcagctggaagagcagcattgccagctttttcttcatcatcggcctgatcatcgggctgtttctggtgctgagagtgggcatccacctgtgcatcaagctgaagcacaccaagaagcggcagatctacaccgacatcgagatgaatcgcctggggaagtaagaattctgcagatatccagcacagtggcggccgctcgagtgtacaaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatcgctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaa

SEQ ID NO: 5 - DNA, 5158 bp, synthetic construct
  misc_feature 1..5158: note = pCMV-VSV-Njco
  source 1..5158: mol_type = other DNA
ctggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatcctgatcagccaccatgctgtcatatctgatctttgccctggctgtgagcccaatcctcggaaagatcgaaatcgtgttcccacaacacaccacaggggactggaagcgcgtgccccacgagtacaactactgcccgacctcagccgacaagaatagccacggcacgcagaccggcatccctgtggagctgaccatgcccaaggggctcacaacgcaccaagtcgaaggcttcatgtgccacagcgctctctggatgacaacctgcgattttcgctggtatggccccaagtacatcacgcacagcatccacaatgaggaaccaaccgactaccagtgcctcgaagccatcaagtcatacaaggatggggtgagcttcaaccccggcttcccgccccaatcatgtggctacggcaccgtgaccgacgccgaggcccacatcgtgaccgtgacaccccactcagtcaaggtggacgagtacacaggcgaatggatcgacccccacttcatcgggggccgctgtaagggccaaatctgcgagaccgtgcacaacagcaccaagtggtttacgtcatcagacggcgaaagcgtgtgcagccaactgtttacgctcgtgggcggcatcttctttagcgacagcgaggagatcaccagcatgggcctcccggagacaggaatccgcagcaactactttccgtacatcagcaccgagggaatctgtaagatgcctttttgccgcaagcagggatataagctgaagaatgacctgtggttccagatcatggacccggacctggacaagaccgtccgcgatctgccccacatcaaggactgtgatctgtcatcaagcatcatcacccccggagaacacgccacggacatcagcctcatcagcgatgtggagcgcatcctcgactacgctctctgccagaacacatggagcaagatcgaaagcggcgaacccatcaccccagtggacctgagctatctcggcccaaagaaccccggcgtggggcccgtgttcaccatcatcaacgggagcctgcactactttacaagcaagtatctgcgcgtggagctcgaaagcccagtcatcccccgcatggaggggaaggtggccgggacccgcatcgtgcgccagctgtgggaccagtggttcccttttggcgaggtggaaatcggccccaacggcgtgctgaagaccaagcaaggatataagttcccgctgcacatcatcgggacgggcgaagtggacagcgatatcaagatggagcgcgtggtcaagcactgggagcacccacacatcgaggctgctcagacctttctcaagaaggacgataccggcgaagtcctgtattacggggatacgggagtgagcaagaaccctgtggagctggtggaaggctggttcagcggatggcgctcaagcctgatgggcgtgctggccgtcatcatcggatttgtgatcctgatgttcctcatcaagctgatcggcgtgctgtcaagcctgttccgccctaagcgccgcccaatctacaagagcgacgtcgagatggcccactttcgctaagaattctgcagatatccagcacagtggcggccgctcgagtgtacaaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatcgctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaa

SEQ ID NO: 6 - DNA, 720 bp, synthetic construct
  misc_feature 1..720: note = eGFP
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 7 - protein, 239 aa, synthetic construct
  REGION 1..239: note = eGFP protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 8 - DNA, 1725 bp, synthetic construct
  misc_feature 1..1725: note = A111-Luciferase
  source 1..1725: mol_type = other DNA
ggatccgccaccatggccgcagccgcagccgtgaagaactggatgactcaaacgctcgccgctgcaatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgag

SEQ ID NO: 9 - protein, 568 aa, synthetic construct
  REGION 1..568: note = A111-Luciferase
MAAAAAVKNWMTQTLAAAMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 10 - DNA, 1026 bp, unidentified
  misc_feature 1..1026: note = circumsporozoite (CS) protein (CSP)
  source 1..1026: mol_type = unassigned DNA; note = Plasmodium berghei
atggccaagaagtgtaccatactggtcgttgcgtcacttctgttggtcaattctctgctcccaggctatggacagaacaaatccattcaggcacagaggaacctcaacgaactctgctacaatgaagggaatgacaataagctgtatcacgtgctgaattccaagaacggcaaaatctacaatcgcaacacagtaaatcggttgcttgccgatgcacccgagggtaagaagaatgaaaagaagaatgagaaaatcgagcgcaacaacaagcttaaacagccaccgcctcctcctaacccaaatgacccaccgccacccaatccaaacgacccaccgcctcccaaccctaacgatcctccaccgcccaaccctaatgacccaccacctcccaatgcaaacgatccaccccctcctaacgctaacgaccctgctccacccaacgctaacgatcccgcgccccccaatgccaacgaccccgcaccacctaatgccaacgatcccgccccgcccaatgctaatgatcctccaccacccaacccaaacgaccctgcccctcctaatgctaacgatccaccacctcccaatccgaatgatcccgctccacctcaggggaacaacaaccctcagccccaacctagaccacagccgcagccccaaccccaaccccagccccagcctcaaccccagccccagccacgtccgcagcctcagcctcaacctggaggcaacaataacaacaagaataacaacaacgacgacagctacattccaagtgccgagaaaattctggagtttgttaagcagatccgagacagcataaccgaagaatggtcacagtgtaacgtgacgtgtggatctggcatcagagtgaggaaacggaagggttccaataagaaagcagaggatctgactctggaggacattgatacagagatctgcaaaatggacaaatgcagctctatcttcaacatcgtgagtaatagcctcgggtttgtgattctgctggtcctggtgttcttcaattga

SEQ ID NO: 11 - protein, 341 aa, unidentified
  REGION 1..341: note = misc_feature - CSP
  source 1..341: note = Plasmodium berghei
MAKKCTILVVASLLLVNSLLPGYGQNKSIQAQRNLNELCYNEGNDNKLYHVLNSKNGKIYNRNTVNRLLADAPEGKKNEKKNEKIERNNKLKQPPPPPNPNDPPPPNPNDPPPPNPNDPPPPNPNDPPPPNANDPPPPNANDPAPPNANDPAPPNANDPAPPNANDPAPPNANDPPPPNPNDPAPPNANDPPPPNPNDPAPPQGNNNPQPQPRPQPQPQPQPQPQPQPQPQPRPQPQPQPGGNNNNKNNNNDDSYIPSAEKILEFVKQIRDSITEEWSQCNVTCGSGIRVRKRKGSNKKAEDLTLEDIDTEICKMDKCSSIFNIVSNSLGFVILLVLVFFN

SEQ ID NO: 12 - protein, 340 aa, unidentified
  REGION 1..340: note = misc_feature - CSP
  source 1..340: note = Plasmodium berghei
MKKCTILVVASLLLVNSLLPGYGQNKSIQAQRNLNELCYNEGNDNKLYHVLNSKNGKIYNRNTVNRLLADAPEGKKNEKKNEKIERNNKLKQPPPPPNPNDPPPPNPNDPPPPNPNDPPPPNPNDPPPPNANDPPPPNANDPAPPNANDPAPPNANDPAPPNANDPAPPNANDPPPPNPNDPAPPNANDPPPPNPNDPAPPQGNNNPQPQPRPQPQPQPQPQPQPQPQPQPRPQPQPQPGGNNNNKNNNNDDSYIPSAEKILEFVKQIRDSITEEWSQCNVTCGSGIRVRKRKGSNKKAEDLTLEDIDTEICKMDKCSSIFNIVSNSLGFVILLVLVFFN

SEQ ID NO: 13 - DNA, 1203 bp, synthetic construct
  misc_feature 1..1203: note = PfCSPhumanCO
  source 1..1203: mol_type = other DNA
gccaccatggctatgagaaagctcgcgatcctttccgtgagctcattcctgtttgtcgaagccttgttccaggagtatcagtgctatggatcatcctccaacacaagagtgctcaacgaactgaactatgacaatgccggtactaacctctacaatgaactggagatgaactactacggcaaacaggagaactggtattccctgaagaagaattccagatcactgggcgaaaacgacgatgggaataatgaggataacgagaagttgcggaaaccaaagcacaagaagttgaaacaacccgccgacggaaaccctgatcctaacgccaacccaaatgtagatcccaacgccaacccaaacgtcgatcccaatgccaatcccaatgttgaccccaatgcaaaccctaatgcaaatcccaatgccaatcccaatgcaaatcctaatgctaatccaaacgccaaccctaacgcgaaccccaacgccaatcctaacgcaaatcctaacgcaaatcctaatgccaaccctaatgcgaacccgaacgctaatcctaacgctaatccgaatgcaaatccaaatgcaaatccgaacgccaatcccaacgtagacccaaatgcaaacccgaacgctaacccgaacgcaaacccaaacgccaatccgaacgctaatcccaatgctaatcccaacgctaaccccaatgccaacccgaatgccaaccccaatgcgaatccaaatgcgaacccaaacgccaatccgaatgcgaatcctaacgctaacccaaatgctaatccaaacgctaatccaaatgcgaaccccaatgcgaatccaaataagaacaatcaggggaatggtcagggccataacatgcctaacgaccccaaccgaaatgtggacgagaacgctaacgcaaacagcgctgtaaagaacaacaacaatgaggagccttctgacaagcacatcaaagagtacctgaataaaatccagaacagtctttctacggaatggtccccatgtagtgttacttgtggcaatgggattcaagtcaggatcaaaccaggctctgcgaataagcctaaggatgaactggattatgccaatgacatcgagaagaaaatatgcaagatggagaagtgcagtagcgtgttcaatgtcgtaaactcaagcataggtctgataatggtactgagctttctgttcctcaactaa

SEQ ID NO: 14 - protein, 398 aa, synthetic construct
  REGION 1..398: note = PfCSP
MAMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 15 - protein, 423 aa, synthetic construct
  REGION 1..423: note = CSP
MRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHITEYLKKIQNSLSTEWSPCSVTCGNGIQVRIKPGSAGKSKDELDYENDIEKKICKMEKCSSVFNVVNSSIGLIMVLSFLFLN

SEQ ID NO: 16 - DNA, 1143 bp, synthetic construct
  misc_feature 1..1143: note = PvCSPhumanCO
  source 1..1143: mol_type = other DNA
gccaccatggctaagaacttcattctgcttgccgtaagctctattctcttggtagacttgtttccaacacactgtggacataatgtagatctcagtaaagcaattaacctcaatggtgttaattttaataatgtagacgcatcctctctcggcgcggcacacgtgggtcaaagtgccagtagggggcgcggactcggcgagaacccggacgacgaagaaggggatgcgaaaaagaagaaggatgggaagaaggcagaacccaagaatccaagggagaataagctgaagcagccaggagatagagctgacggccagcctgccggcgatagggccgacggacaacccgcgggagaccgcgcggatggccaacctgcgggtgaccgcgctgacgggcagcccgcaggagacagagccgccggacaacccgccggcgaccgagcggatggtcagcccgcgggcgatcgagcggacggtcagccagctggagatcgcgcggacggacaacctgcgggggacagggctgacggtcaaccagccggtgatagagcagcgggccaaccggcaggggaccgagccgcagggcagcccgccggggacagggctgacgggcagccagcgggggatagggccgccggtcaacctgcaggcgatcgagctgacggccaacccgcgggtgaccgggcagccggccaacctgctggggatcgagcagacggacagccggccggagatcgggccgctggccaaccggctggagacagagccgctggtcaacctgccggagatcgggccgctgggcaggccgcgggtgacagggcagccggacaagcagcaggggggaacgccggtggacaaggacagaacaatgagggtgcgaatgccccgaatgaaaagagcgttaaagagtacctggataaggttagagcaaccgtaggcaccgaatggaccccctgctccgtgacttgcggggtaggtgttcgggtcaggcggagggtcaatgcggcgaacaagaaaccggaagatctgactttgaacgatcttgaaacagatgtatgcacgatggacaagtgtgctggcatatttaacgtagtcagtaactctctcggccttgtgatcctcttggtcttggcgctgtttaactag

SEQ ID NO: 17 - protein, 378 aa, unidentified
  REGION 1..378: note = misc_feature - PvCSP
  source 1..378: note = Plasmodium vivax
MAKNFILLAVSSILLVDLFPTHCGHNVDLSKAINLNGVNFNNVDASSLGAAHVGQSASRGRGLGENPDDEEGDAKKKKDGKKAEPKNPRENKLKQPGDRADGQPAGDRADGQPAGDRADGQPAGDRADGQPAGDRAAGQPAGDRADGQPAGDRADGQPAGDRADGQPAGDRADGQPAGDRAAGQPAGDRAAGQPAGDRADGQPAGDRAAGQPAGDRADGQPAGDRAAGQPAGDRADGQPAGDRAAGQPAGDRAAGQPAGDRAAGQAAGDRAAGQAAGGNAGGQGQNNEGANAPNEKSVKEYLDKVRATVGTEWTPCSVTCGVGVRVRRRVNAANKKPEDLTLNDLETDVCTMDKCAGIFNVVSNSLGLVILLVLALFN

SEQ ID NO: 18 - protein, 368 aa, unidentified
  REGION 1..368: note = misc_feature - CSP
  source 1..368: note = Plasmodium vivax
MKNFILLAVSSILLVDLFPTHCGHNVDLSKAINLNGVNFNNVDASSLGAAHVGQSASRGRGLGENPDDEEGDAKKKKDGKKAEPKNPRENKLKQPGDRADGQPAGDRADGQPAGDRADGQPAGDRAGQPAGDRADGQPAGDRADGQPAGDRADGQPAGDRADGQPAGDRAGQPAGDRAGQPAGDRADGQPAGDRAGQPAGDRADGQPAGDRAGQPAGDRADGQPAGDRAGQPAGDRAGQPAGDRAGQPAGDRAGQPAGNGAGGQAAGGNAGGQGQNNEGANAPNEKSVKEYLDKVRATVGTEWTPCSVTCGVGVRVRRRVNAANKKPEDLTLNDLETDVCTMDKCAGIFNVVSNSLGLVILLVLALFN

SEQ ID NO: 19 - DNA, 1842 bp, unidentified
  misc_feature 1..1842: note = thrombospondin-related anonymous protein (TRAP)
  source 1..1842: mol_type = unassigned DNA; note = Plasmodium berghei
ggatccgccaccatggctaagcttctggggaacagtaaatacttctttgtggtcctgctgctgtgcattagcgttttcctcaacggtcaggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggttctccatcccaactgtgccggggaaatgacagccccatgtaaagtgcgggactgtcctcccaaacctgtagcccctccggtcattcccatcaaagtccctgacgtgcctgtgaaaccagtcgaacctattgagcccgccgagccagcagagccagcagaaccagcagagcctgcagaacccgccgaacccgctgagcccgcggagcccgccgaacccgctgaaccggcagaacccgcggaaccagcggagcctgcagagccagctgagcctgctgaaccggcggagcccgctgaaccagccgagcctgctaaaccggccgaaccggcagagcccgctgagcctgccgagccagcggaaccagttaaccccgataatcctatcctgccgatcaagcccgaggagccatctggtggagccgagccattgaatccagaggtcgagaatccctttatcatccccgacgaacccatcgaacccattattgcgccaggagctgtaccggataagcccatcattcctgaggaatcaaatgagctgccaaacaatcttccagagtctccctccgatagtcaggtggagtatcctcggccaaacgacaatggggataacagcaacaacacaatcaattccaacaagaacataccaaataagcatgtgcctcctacagacgacaacccctacaagggccaggaagaacgaatccctaagccgcatcggagcaacgacgaatacatttactacaataatgctaacaataacgacaagctggagcccgagataccctctaaggattacgaggaaaacaagagcaagaaacagagcaaaagcaacaatggctataagatcgccggcggcataattggcgggctggctattatcggctgcattggagtgggctataacttcatagccgggtcctccgccgccgctatggctggagaggcggcaccttttgaggacgtgatggctgatgatgagaaggggatcgtggaaaacgaacagttcaaactgccagaggacaatgattggaattgactcgag

SEQ ID NO: 20 - protein, 607 aa, unidentified
  REGION 1..607: note = misc_feature - TRAP
  source 1..607: note = Plasmodium berghei
MAKLLGNSKYFFVVLLLCISVFLNGQEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKVLHPNCAGEMTAPCKVRDCPPKPVAPPVIPIKVPDVPVKPVEPIEPAEPAEPAEPAEPAEPAEPAEPAEPAEPAEPAEPAEPAEPAEPAEPAEPAEPAEPAEPAKPAEPAEPAEPAEPAEPVNPDNPILPIKPEEPSGGAEPLNPEVENPFIIPDEPIEPIIAPGAVPDKPIIPEESNELPNNLPESPSDSQVEYPRPNDNGDNSNNTINSNKNIPNKHVPPTDDNPYKGQEERIPKPHRSNDEYIYYNNANNNDKLEPEIPSKDYEENKSKKQSKSNNGYKIAGGIIGGLAIIGCIGVGYNFIAGSSAAAMAGEAAPFEDVMADDEKGIVENEQFKLPEDNDWN

SEQ ID NO: 21 - protein, 606 aa, unidentified
  REGION 1..606: note = misc_feature - TRAP
  source 1..606: note = Plasmodium berghei
MKLLGNSKYFFVVLLLCISVFLNGQEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKVLHPNCAGEMTAPCKVRDCPPKPVAPPVIPIKVPDVPVKPVEPIEPAEPAEPAEPAEPAEPAEPAEPAEPAEPAEPAEPAEPAEPAEPAEPAEPAEPAEPAEPAKPAEPAEPAEPAEPAEPVNPDNPILPIKPEEPSGGAEPLNPEVENPFIIPDEPIEPIIAPGAVPDKPIIPEESNELPNNLPESPSDSQVEYPRPNDNGDNSNNTINSNKNIPNKHVPPTDDNPYKGQEERIPKPHRSNDEYIYYNNANNNDKLEPEIPSKDYEENKSKKQSKSNNGYKIAGGIIGGLAIIGCIGVGYNFIAGSSAAAMAGEAAPFEDVMADDEKGIVENEQFKLPEDNDWN

SEQ ID NO: 22 - DNA, 1734 bp, synthetic construct
  misc_feature 1..1734: note = thrombospondin-related anonymous protein (TRAP)
  source 1..1734: mol_type = other DNA
gccaccatggctaatcacttgggcaacgtgaagtacctggtgatagtgttcctgatcttcttcgacctgtttctggtcaacggcagagatgtgcagaacaacatagtcgacgagattaagtacagagaggaggtgtgcaatgacgaggtggatctctacctgctgatggactgtagcgggtcaatacgacggcacaattgggttaatcatgctgtccccttggctatgaagctgatccagcagctcaatctgaacgataatgccattcacctgtatgcttccgtgttcagcaataatgcaagggagatcattcgcctccacagtgacgctagtaagaataaggagaaagccctgatcatcatcaaatccctgcttagcaccaaccttccctatggcaagaccaatctcacagacgcgcttctgcaggtaaggaagcatctgaacgatcgcatcaacagagagaatgcgaatcagctggtagtaatcctgactgatgggattcccgattccatccaggacagcctgaaggaatcaaggaagctcagcgatagaggggtgaaaatcgcagttttcggaatcggtcaggggattaacgtggcctttaatcgctttctggtgggatgtcatccatctgatgggaaatgcaatctgtatgccgattctgcgtgggagaatgtgaagaacgtgattggcccctttatgaaagccgtgtgcgtagaggtggagaaaaccgcctcctgtggagtttgggacgaatggagcccctgttccgttacttgcgggaaagggacccgaagtcgcaagagagaaatccttcacgagggttgcacttctgaactccaagagcaatgcgaggaggaaaggtgtctccctaaacgagaaccccttgatgtgcctgacgaacctgaggacgatcaaccaagaccaaggggagacaacttcgccgtcgagaaacccaatgagaatatcattgacaacaatccacaggagccatcacctaatccagaggaaggcaaaggcgaaaaccctaacggttttgacttggacgagaatccggaaaatccacccaacccgcctaaccctccaaatccaccaaaccctccgaatcctcctaaccctgatatccccgagcaagagcctaatattccagaggatagcgaaaaggaggtcccctctgacgtccctaagaaccctgaagatgatcgcgaagagaactttgacatacccaagaaacccgagaataaacacgataatcagaacaatctcccgaacgataaatctgaccgctacattccatacagtccattgagcccaaaggtcttggataatgaacggaaacagtccgaccctcaatcacaggacaataacggtaatcgtcatgttccgaactccgaagatcgggaaacaaggccccatgggcgtaacaacgaaaaccggagctataaccggaagcacaacaacacacccaagcacccggaacgggaagaacatgagaagcccgataacaacaagaagaaggcaggctctgacaacaaatacaaaatagccggaggtattgccggaggattggcactgctggcctgcgctggccttgcttacaagttcgtcgttccaggcgccgcaacgccctatgcaggcgaaccagctccctttgacgagacactgggagaagaggacaaagacctggacgagcctgaacagttcagactgcccgaggaaaacgagtggaattaa

SEQ ID NO: 23 - protein, 575 aa, synthetic construct
  REGION 1..575: note = PfTRAP
MANHLGNVKYLVIVFLIFFDLFLVNGRDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCLPKREPLDVPDEPEDDQPRPRGDNFAVEKPNENIIDNNPQEPSPNPEEGKGENPNGFDLDENPENPPNPPNPPNPPNPPNPPNPDIPEQEPNIPEDSEKEVPSDVPKNPEDDREENFDIPKKPENKHDNQNNLPNDKSDRYIPYSPLSPKVLDNERKQSDPQSQDNNGNRHVPNSEDRETRPHGRNNENRSYNRKHNNTPKHPEREEHEKPDNNKKKAGSDNKYKIAGGIAGGLALLACAGLAYKFVVPGAATPYAGEPAPFDETLGEEDKDLDEPEQFRLPEENEWN

SEQ ID NO: 24 - protein, 560 aa, synthetic construct
  REGION 1..560: note = TRAP
MNHLGNVKYLVIVFLIFFDLFLVNGRDVQNNIVDEIKYREEVCNDEVDLYLLMDCSGSIRRHNWVNHAVPLAMKLIQQLNLNENAIHLYANVFSNNAREIIRLHSDASKNKEKALSIIKSLLSTNLPFGRTNLTDALLQVRKHLNDRINRENANQLVVILTDGIPNSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCPPKWEPLDVPDDPEDDQPRPRGDNSSVQKPEENIIDNNPQEPSPNPEEGKGENPNGFDLDENPENPPNPDIPQQEPNIPEDSEKEVPSDVPKNPEDDREENFDIPKKPENKHDNQNNLPNDKSDRYIPYSPLPPNVLDNERKQSDPQSQDNNGNRHVPNSEDRETRPHGRNNENRSYNRKHNDTPKHPEREEHEKPDNNKKKGGSSDNKYKIAGGIAGGLALLACAGLAYKFVVPGAATPYAGEPAPFDETLGEEDKDLDEPEQFRLPEENEWN

SEQ ID NO: 25 - DNA, 1680 bp, synthetic construct
  misc_feature 1..1680: note = PvTRAPhumanCO
  source 1..1680: mol_type = other DNA
gccaccatggctaagctgctgcagaacaagtcttatctcctggtcgtgttcctgctttacgtatccatatttgcacggggagatgaaaaagttgtagatgaggtaaagtacagcgaggaggtatgcaacgaatctgtcgatctctatcttctcgtagatggctctggtagtattgggtatccgaactggataactaaggtaattcctatgctgaacggtcttattaatagtttgagcctcagtcgagacacgatcaatctctatatgaatctcttcggaaattacactacagaactcatacggctgggctctgggcagagtatagataaacgacaggctctgtcaaaggtgactgaacttagaaagacctacacgccatatggcactactaacatgacagccgcgcttgatgaagtccaaaagcaccttaatgatagagtgaaccgagagaaagcgatacaacttgtcatattgatgaccgacggggtgcccaactcaaaatacagggcgctggaggtcgcaaataaactgaagcaacgcaatgtttccttggccgttattggtgtcggacaaggaatcaaccatcagttcaatcggctgatcgcgggctgccgacctcgggagccgaactgcaaattttactcttatgctgattggaatgaggccgtagcacttataaagccctttattgctaaggtatgcacagaagttgagcgcgttgccaactgtggaccctgggacccctggaccgcgtgtagcgttacgtgcggcaggggaacgcactccaggtctcgcccttcattgcatgaaaagtgcactacccatatggtgtctgaatgtgaagagggggaatgcccagtggagcctgagccactgccagtacctgcgccactcccaacggttccagaagatgtaaatccgcgagacacggacgacgaaaatgagaacccgaacttcaacaagggactggacgtgccggatgaagatgatgacgaagtaccgcccgcgaatgaaggtgcggatggcaacccagtcgaggaaaatgttttcccgcctgctgacgatagcgttcctgatgagagtaacgtgctgcctctccctccggcagtcccaggcgggtcctctgaggagttcccggcggacgttcagaacaatcccgactctcctgaagagctgccgatggagcaagaagtgcctcaagataataacgtgaatgaaccagagagatccgatagcaacggttatggcgtaaatgagaaggtgatacctaatcccctcgacaatgagcgagacatggccaataagaataagaccgttcacccaggccggaaggacagcgcgagagatcgatatgcccgcccccatggttccactcatgtcaataataatagagccaatgagaactcagacataccaaacaacccagtaccttctgattacgaacagccggaggacaaggctaaaaagtcctccaataacggctataaaatcgctggtggagtgatcgctgggctcgcgttggttgggtgcgtcggtttcgcgtataacttcgtagcgggtggaggcgctgctggtatggcaggcgaaccggctccctttgacgaagctatggccgaagacgaaaaagacgtagccgaagcggaccagttcaaattgcctgaggacaacgattggaattaa

SEQ ID NO: 26 - protein, 557 aa, unidentified
  REGION 1..557: note = misc_feature - PvTRAP
  source 1..557: note = Plasmodium vivax
MAKLLQNKSYLLVVFLLYVSIFARGDEKVVDEVKYSEEVCNESVDLYLLVDGSGSIGYPNWITKVIPMLNGLINSLSLSRDTINLYMNLFGNYTTELIRLGSGQSIDKRQALSKVTELRKTYTPYGTTNMTAALDEVQKHLNDRVNREKAIQLVILMTDGVPNSKYRALEVANKLKQRNVSLAVIGVGQGINHQFNRLIAGCRPREPNCKFYSYADWNEAVALIKPFIAKVCTEVERVANCGPWDPWTACSVTCGRGTHSRSRPSLHEKCTTHMVSECEEGECPVEPEPLPVPAPLPTVPEDVNPRDTDDENENPNFNKGLDVPDEDDDEVPPANEGADGNPVEENVFPPADDSVPDESNVLPLPPAVPGGSSEEFPADVQNNPDSPEELPMEQEVPQDNNVNEPERSDSNGYGVNEKVIPNPLDNERDMANKNKTVHPGRKDSARDRYARPHGSTHVNNNRANENSDIPNNPVPSDYEQPEDKAKKSSNNGYKIAGGVIAGLALVGCVGFAYNFVAGGGAAGMAGEPAPFDEAMAEDEKDVAEADQFKLPEDNDWN

SEQ ID NO: 27 - protein, 556 aa, unidentified
  REGION 1..556: note = misc_feature - TRAP
  source 1..556: note = Plasmodium vivax
MKLLQNKSYLLVVFLLYVSIFARGDEKVVDEVKYSEEVCNESVDLYLLVDGSGSIGYPNWITKVIPMLNGLINSLSLSRDTINLYMNLFGNYTTELIRLGSGQSIDKRQALSKVTELRKSYSPYGTTNMTAALDEVQKHLNDRVNREKAIQLVILMTDGIPNSKYTALEVAKKLKQRNVSLAVIGIGQGINHQFNRLIAGCRPRESNCKFYSYADWNEAVALIKPFIAKVCTEVERVANCGPWDPWTACSVTCGRGTHSRSRPSLHEGCTTHMVSECEEGECPVEPEPLPVPAPLPTVPEDVNPRDTDDENENPNFNKGLDVPEEDDDEVPPANERADGNPVEENVFPPADDSVPDESNVLPLPPAVPGGSSEEFPADVQNNPDSPEELPMEQEVPQDNNVNEPERSDSNGYGVNEKVIPNPLDNERDMANKNKTVHPDRKDSARDRYARPHGSTHVNNNRANENSDIPNNPVPSDYEQPEDKAKKSSNNGYKIAGGVIAGLALVGCVGFAYNFVAGGGAAGMAGEPAPFDEAMAEDEKDVAEADQFKLPEDNDWN

SEQ ID NO: 28 - DNA, 1086 bp, synthetic construct
  misc_feature 1..1086: note = inhibitor of cysteine proteases (ICP)
  source 1..1086: mol_type = other DNA
ggatccgccaccatggctaaatccattaccttctttgtgttcaacatttgcagcattttggctctgctgagtcactgtgaggacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattccaatatcgcctttaacgaagagtccgaatacattgagattaatagcgagtctgacttggagaacaagatcaaggacatcaacatcaaatccaatcttgaggaaaacaataccatgaacgaatccggcaaagtggatagcaagtatgagctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacagtgactcgag

SEQ ID NO: 29 - protein, 355 aa, unidentified
  REGION 1..355: note = misc_feature - ICP
  source 1..355: note = Plasmodium berghei
MAKSITFFVFNICSILALLSHCEDNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNSNIAFNEESEYIEINSESDLENKIKDINIKSNLEENNTMNESGKVDSKYELTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQ

SEQ ID NO: 30 - protein, 354 aa, unidentified
  REGION 1..354: note = misc_feature - ICP
  source 1..354: note = Plasmodium berghei
MKSITFFVFNICSILALLSHCEDNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNSNIAFNEESEYIEINSESDLENKIKDINIKSNLEENNTMNESGKVDSKYELTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQ

SEQ ID NO: 31 - DNA, 1251 bp, synthetic construct
  misc_feature 1..1251: note = PfICPhumanCO
  source 1..1251: mol_type = other DNA
gccaccatggctaatctgttggtgttcttttgcttctttctgttgtcttgcatcgtacacctctctcgttgttctgacaacaattcatatagctttgagatcgtgaatcggagcacatggctcaacattgctgaacggatattcaaaggcaatgcaccattcaactttactattataccgtacaactatgtgaacaatagtactgaagagaataataacaaagactctgtccttctgataagcaagaatctgaagaattcctcaaacccagtggacgaaaataaccacatcattgactcaaccaagaagaacacgtccaataacaacaataataacagcaatatcgtcggaatatacgaaagtcaggtacatgaggagaagattaaagaggacaatacaagacaggataatatcaacaagaaggaaaacgagataatcaacaataaccatcagatccctgtgtccaacatcttttcagagaacattgacaacaacaagaactacattgagagcaactacaagagcacctataacaataatcccgagttgattcatagcacagatttcattggcagtaataacaaccacactttcaatttcctgtctcgctataacaactcagtgctgaacaacatgcaagggaataccaaagttccagggaatgttcccgaactgaaagcccgcattttctccgaggaagaaaacaccgaagtcgaaagcgccgaaaacaatcacactaacagtctgaatcctaacgagtcttgtgatcaaatcatcaaactgggcgatatcattaacagcgtcaatgagaagatcatcagcatcaatagtacggtgaataacgtgctctgcataaatctggattccgtcaatggcaatggcttcgtttggacccttcttggggtacacaagaagaaacccctgattgacccctccaattttcccactaaaagggtgactcagtcctacgtttcacctgacatttccgttacaaaccctgtgccaattccaaagaacagcaacaccaacaaagatgacagcatcaataataaacaggatggttcccagaataacacaacaacgaatcactttccgaagcctagagagcaactcgtgggtggatcttctatgctgatcagtaagatcaaaccccataaacccggaaagtatttcatcgtgtatagctactacagaccatttgaccctacaagggataccaacacccgaattgtggagctgaatgtccagtaa

SEQ ID NO: 32 - protein, 414 aa, unidentified
  REGION 1..414: note = misc_feature - PfICP
  source 1..414: note = Plasmodium falciparum
MANLLVFFCFFLLSCIVHLSRCSDNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQ

SEQ ID NO: 33 - protein, 413 aa, unidentified
  REGION 1..413: note = misc_feature - ICP
  source 1..413: note = Plasmodium falciparum
MNLLVFFCFFLLSCIVHLSRCSDNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQ

SEQ ID NO: 34 - DNA, 1104 bp, synthetic construct
  misc_feature 1..1104: note = PvICPhumanCO
  source 1..1104: mol_type = other DNA
gccaccatggctaagttgtctagcctgttctgcctggttgtgtgttctagtgttgcccacctctcttcctgtagtgatcaaaacacttatagttttgatattgttaatcgaaacacttggtacagcatagccaagaaaatctttcaaggcacgaccccctgtaatttcactgtaatccctagttcctatgtcaacaactctgacggagtgtctacgagtgatgattccgtactgctcatccgcaaaaagctcaaggatccgagtgaagctggccttgacggatcttcagtttctggttcatccagttctggaaacagtcactccggttctgcaccttgttgtgataagggtacccccgctaaagaggcagagctgaaattttctacaaagtttgagggcgatgactatgctaagctgagagattctctgagccttatagacaagtcactccgagaagagtcaagctcagaggaggacagtaagatggaagatagtcaggtcggtgaagtaactcatgaagagactatcacctacaacatgcccgaagaatatatgccccagaacatttccgaggtattgatcggtgccgctgaagaggataggacatacgcgttgaagggggacgagccgtgtgatgtgtacttgaaacttggcgagataatcaatggaactaatgaaaagactatcgagtattctctccaaaaaaataagatactgtgcgttcaactcgaagcaattgggggaaatggatacctctgggctctcctcggcgtacacaaagaaaaaccccaaatcaacccagaggagtttccacgaaaaaagatcacaaaatcttttttcaccaatgagatatccgtcacgcagcctaaagcagtgcaaaagaacaaatctaataatggaggtgagagcagttcaaactcacctggctatgggaaaccccccgcaagcgaacagctcgggggatttgtgggtggcacatccatgcttcagagtatagtaaaggctcataaagagggcacctttttcgtagtttatagctactaccgccccttcgaccctaccgccaacgccaacacgaagatactcaaacttacggtttcatag

SEQ ID NO: 35 - protein, 365 aa, synthetic construct
  REGION 1..365: note = PVICP humanco + kozac
  REGION 1..365: note = misc_feature - PvICP
MAKLSSLFCLVVCSSVAHLSSCSDQNTYSFDIVNRNTWYSIAKKIFQGTTPCNFTVIPSSYVNNSDGVSTSDDSVLLIRKKLKDPSEAGLDGSSVSGSSSSGNSHSGSAPCCDKGTPAKEAELKFSTKFEGDDYAKLRDSLSLIDKSLREESSSEEDSKMEDSQVGEVTHEETITYNMPEEYMPQNISEVLIGAAEEDRTYALKGDEPCDVYLKLGEIINGTNEKTIEYSLQKNKILCVQLEAIGGNGYLWALLGVHKEKPQINPEEFPRKKITKSFFTNEISVTQPKAVQKNKSNNGGESSSNSPGYGKPPASEQLGGFVGGTSMLQSIVKAHKEGTFFVVYSYYRPFDPTANANTKILKLTVS

SEQ ID NO: 36 - protein, 359 aa, unidentified
  REGION 1..359: note = misc_feature - ICP
  source 1..359: note = Plasmodium vivax
MKLSSLFCLVVCSSVAHLSSCSDQNTYSFDIVNRNTWYSIAKKIFQGTTPCNFTVIPSSYVNNSDGVSTSDDSVLLIRKKLKDPSEAGLDGSSVSGNSHSGSAPCCDKGTPAKEAELNFSTKFEGDDYAKLRDSLSLIDKSLREESSSEEDSKMEDSQVGEVTHEETITYNMPEEYMPQNISEVLIGAAEEDRTYALKGDEPCDVYLKLGEIINGTNEKTIEYSLQKNKILCVQLEAIGGNGYLWALLGVHKEKPQINPEEFPRKKITKSFFTNEISVTQPKAVQKNKSNNGGESSSNSPGYGKPPASEQLGGFVGGTSMLQSIVKAHKEGTFFVVYSYYRPFDPTANANTKILKLTVS

SEQ ID NO: 37 - DNA, 2355 bp, synthetic construct
  misc_feature 1..2355: note = Falcilysin, Bergheilysin-A-CO
  source 1..2355: mol_type = other DNA
ggatccgccaccatggctaaattgatgaaggttttgggttacatcaacattattaccaactgcgtgaatggaatcctctgcaaaggtgacaagaaaaggtactccatcttcaccaataattacatctacagcatatcaaccctcaataattatagctttgctgcaacaatgaacaaaatgcccgcttgggttaacgagaaatgtccagaacacaaaagctacgacatcgttgagaagcgctacaatgagaacctcaatctcacgtacacagtctatgagcacaagaaggccaagactcaggtcatagcactggggtctaacgatcctctcgacgctgagcaagcatttggcttctacgtgaaaaccctgacgcattcagataaaggcattccgcacatactggaacacactgtcctgagtggctctaagaatttcaactacaaggactcaatggggcttttggagaaaggcaccctgaacacacacctgaatgcctacaccttcaatgacaggaccatctacatggccgggagtatgaacaatagggatttctttaacattatggccgtctacatggatagcgtgttccagcctaacgtactggaaaacaaattcatcttccagacagagggatggacctatgaggtagagaagctgaaggaggaggagaagaacctcgacattcccaagattaaggactacaaggtgtcttttaacggaatcgtgtataatgagatgaagggtgcgtttagcaatcctctgcaggacctgtattatgaagtgatgagaaacatgttccccgacaacgtacacagtaacatctccggaggagatcctaaagaaataccaaatctgtcatatgaagagtttaaggagttttactacaagaattacaatccgaagaaaatcaaagtgttctttttctccaaaaataatccgacagagctgctcaactttgtggacaactatctctgtcagctggacttcacgaaatatcgggatgatgctgtggaacatgtcaattaccaagaataccgcaaaggcccattctatattaagaagaaattcgctgatcactcagaagagaaagaaaatcttgcctccgtgagttggcttctgaatcccaagaaacacaagaattccgatactgatctctctctggagtctcctacagactatttcgccttgctcatcatcaataatctgcttactcataccagtgagagcgtcctgtacaaagccctgatagaatccggattggggaatagtattgtagatcgagggctgaatgattccctggttcagtatgtgttcagcatcggcctgaaaggcataaaagagaagaacgagaagaacatctccctggacaaggtccactacgaggtggaaaagatcgttcttgaggcactgaaaaaagtggtcaaagaaggtttcaataagtcagcagttgaggcagccattaacaatattgagttcgtcctgaaagaagccaatctcaagatctccaaatctatagactttgtgtttgaaatggccagcagactgaactatggcaaagatccactgctgatctttgagtttgaaaagcatctcaacgtagtgaaggacaagatcaagaacgagcctaaatacctggagaagtatgtggagaaacatcttctgaacaatgatcatcgagtcgttattctgctggaaggggatgaaaactatggcaccgaacaggagaaactggaaaaggacatgctgaagaagcggattgaaagcttcactgagaaagaaaaggagaatattatcacagacttcgaaaatcttacgaagtacaagaacactgaggaatctcccgaacatctggacaagtttcccatcattagcattagcgacctcaatggaaagactttggagatccccgtgaaccctttctttaccaacctgaacaacgagaacaacatgcagcactataatgagacaaagaacaaccaaaccctggtcaaagaaaacatggaccgtttcattaacaaatacattctcaacaaggatggaaacgataagaacgacagcaagaacgcggatgtgccaatgctgatttatgaaatcccaacatctggcatattgtatctgcagtttatcttctctctggataaccttacactggaagaattgtcctacctgaatctgtttaaaagcttgatactggagaacaagactaacaagagatcaagtgaagagtttgtgatcttgcgggagaagaatattgggaatatgatgaccaatgttgctcttcttagcacatccgatcgcctcaatgtgactgacaaatataatgcgaaaggtttctttaactttgagtaactcgag

SEQ ID NO: 38 - protein, 778 aa, unidentified
  REGION 1..778: note = misc_feature - Falcilysin, Bergheilysin-A
  source 1..778: note = Plasmodium berghei
MAKLMKVLGYINIITNCVNGILCKGDKKRYSIFTNNYIYSISTLNNYSFAATMNKMPAWVNEKCPEHKSYDIVEKRYNENLNLTYTVYEHKKAKTQVIALGSNDPLDAEQAFGFYVKTLTHSDKGIPHILEHTVLSGSKNFNYKDSMGLLEKGTLNTHLNAYTFNDRTIYMAGSMNNRDFFNIMAVYMDSVFQPNVLENKFIFQTEGWTYEVEKLKEEEKNLDIPKIKDYKVSFNGIVYNEMKGAFSNPLQDLYYEVMRNMFPDNVHSNISGGDPKEIPNLSYEEFKEFYYKNYNPKKIKVFFFSKNNPTELLNFVDNYLCQLDFTKYRDDAVEHVNYQEYRKGPFYIKKKFADHSEEKENLASVSWLLNPKKHKNSDTDLSLESPTDYFALLIINNLLTHTSESVLYKALIESGLGNSIVDRGLNDSLVQYVFSIGLKGIKEKNEKNISLDKVHYEVEKIVLEALKKVVKEGFNKSAVEAAINNIEFVLKEANLKISKSIDFVFEMASRLNYGKDPLLIFEFEKHLNVVKDKIKNEPKYLEKYVEKHLLNNDHRVVILLEGDENYGTEQEKLEKDMLKKRIESFTEKEKENIITDFENLTKYKNTEESPEHLDKFPIISISDLNGKTLEIPVNPFFTNLNNENNMQHYNETKNNQTLVKENMDRFINKYILNKDGNDKNDSKNADVPMLIYEIPTSGILYLQFIFSLDNLTLEELSYLNLFKSLILENKTNKRSSEEFVILREKNIGNMMTNVALLSTSDRLNVTDKYNAKGFFNFE

SEQ ID NO: 39 - protein, 1149 aa, unidentified
  REGION 1..1149: note = misc_feature - Falcilysin, Bergheilysin-A
  source 1..1149: note = Plasmodium berghei
MKLMKVLGYINIITNCVNGILCKGDKKRYSIFTNNYIYSISTLNNYSFAATMNKMPAWVNEKCPEHKSYDIVEKRYNENLNLTYTVYEHKKAKTQVIALGSNDPLDAEQAFGFYVKTLTHSDKGIPHILEHTVLSGSKNFNYKDSMGLLEKGTLNTHLNAYTFNDRTIYMAGSMNNRDFFNIMAVYMDSVFQPNVLENKFIFQTEGWTYEVEKLKEEEKNLDIPKIKDYKVSFNGIVYNEMKGAFSNPLQDLYYEVMRNMFPDNVHSNISGGDPKEIPNLSYEEFKEFYYKNYNPKKIKVFFFSKNNPTELLNFVDNYLCQLDFTKYRDDAVEHVNYQEYRKGPFYIKKKFADHSEEKENLASVSWLLNPKKHKNSDTDLSLESPTDYFALLIINNLLTHTSESVLYKALIESGLGNSIVDRGLNDSLVQYVFSIGLKGIKEKNEKNISLDKVHYEVEKIVLEALKKVVKEGFNKSAVEAAINNIEFVLKEANLKISKSIDFVFEMASRLNYGKDPLLIFEFEKHLNVVKDKIKNEPKYLEKYVEKHLLNNDHRVVILLEGDENYGTEQEKLEKDMLKKRIESFTEKEKENIITDFENLTKYKNTEESPEHLDKFPIISISDLNGKTLEIPVNPFFTNLNNENNMQHYNETKNNQTLVKENMDRFINKYILNKDGNDKNDSKNADVPMLIYEIPTSGILYLQFIFSLDNLTLEELSYLNLFKSLILENKTNKRSSEEFVILREKNIGNMMTNVALLSTSDRLNVTDKYNAKGFFNFEMHMLSHKCNDALEIALEALKESDFSNKKKVIEILKRKINGMKTTFASKGHSILIKYVKSRINSKYYAYDLIHGYDNYLKLQEQLKLAETNYESLEAILNRIRKKIFKRNNLIMNVTVDPGTIDQLFAKSKNSFNNLLSYFDENESYCSKNDSFNKVVGWNKEIQEKKLLEGEEVKKELLVVPTFVNSVSMSGVLFNKGEYLDPSFTVIVAALKNSYLWETVRGLNGAYGVFADIEYDGTVVFLSARDPNLEKTLQTFREAAQGLRKMADVMTKNDLLRYIINAIGTIDRPRRGVELSKLSFSRIISNETEQDRIEFRNRVMNTKKEDFYKFADLLEKKVKEFEKNVVIITSKEKANEYINNVDNDFKKILIE

SEQ ID NO: 40 - DNA, 3591 bp, synthetic construct
  misc_feature 1..3591: note = PfFalcilysin Human CO
  source 1..3591: mol_type = other DNA
gccaccatggctaatcttaccaaacttatgaaagtaataggatatataaacattataactaattgtgtacaatcttttactaacagagcggacaagaagaggtacaatgtttttgcaaagtctttcatcaatacgataaacacgaacctgtacaccttcaaagcggtgatgtcaaaaacgcctgaatggatccatgagaaatctcccaaacacaacagttatgacataattgagaaacgctataatgaagagttcaaaatgacatacacggtttatcaacataaaaaagcaaagacgcaggtaatttcactcggtacgaacgacccacttgatgtcgaacaggcctttgccttctatgtaaagactctgacccactctgggaaggggattccccacatcctcgaacacagtgtcctctcagggagtaaaaattacaactacaaaaattccattggactccttgagaagggaactctgcacacccaccttaacgcatacaccttcaatgaccgaactgtgtacatggccggttctatgaataacaaagacttcttcaatataatgggcgtctatatggacagtgtcttccaacctaatgtactggaaaacaaatacatattcgagacggagggatggacttacgaggtggaaaagctgaaagaggatgagaagggaaaagctgagattccacagatgaaagattataaagtatctttcaacggaattgtttataatgaaatgaagggtgccttgtcttccccgttggaagatctttaccatgaagagatgaagtatatgttcccagacaacgtccactctaataacagtggcggagacccaaaagagatcacaaacttgacctatgaggagttcaaggagttctattacaaaaactataatccaaaaaaagttaaagtgttcttcttttcaaagaacaacccaacggagcttctcaatttcgtagaccagtacctcggacagctggactacagcaaataccgcgacgatgctgttgaaagtgttgaataccaaacgtataaaaaaggacctttctacattaaaaaaaagtatggggaccatagcgaagagaaggagaatcttgtttccgtagcgtggcttctgaaccccaaggttgacaagactaacaatcataataataaccatagtaataaccaatctagcgaaaataatggttactccaacggctcccactctagcgatttgtccttggagaatcccacggactatttcgtgctcttgattatcaataacctccttatacataccccagaaagcgtcctgtacaaggccctcacagattgcgggctgggaaataatgtaattgataggggtttgaatgattcacttgtccaatacattttcagtattgggctgaagggaatcaaacgcaacaatgaaaaaattaaaaacttcgataaagtgcactatgaagtagaagacgtaattatgaatgctctcaaaaaagtggtcaaggagggattcaataaatccgccgtagaagccagcataaataatatcgagtttatcctgaaagaagccaatttgaaaacttcaaaatctatagatttcgtttttgagatgacttccaagctcaattataatagggatccactgctgatcttcgagtttgagaaatatcttaatattgtgaagaataagattaagaacgaacctatgtatttggagaagtttgttgaaaaacacttcatcaacaacgcacatcggtcagtgatccttcttgagggggacgagaactatgcacaggaacaggaaaaccttgagaaacaagaactgaagaaacgcatagagaacttcaatgagcaggagaaagagcaagtcattaagaacttcgaggagctgtccaagtacaagaacgcggaagagagcccggaacacttgaacaagtttccaataatctccatttccgacctcaataagaaaacactggaagtcccagttaacgtctacttcacgaacatcaacgaaaacaacaatataatggagacatataacaagctgaagacaaatgagcacatgcttaaggacaacatggacgtgtttctcaaaaaatacgttctcaaaaacgataagcacaacaccaacaataataacaacaataataataatatggactactctttcaccgaaactaaatatgaaggaaatgtgccaatccttgtgtacgaaatgccgacgactggaatagtctatttgcagttcgttttctccctcgatcacctgaccgtagacgagctcgcctatttgaatttgttcaagacacttatcttggagaacaagacaaacaaacgctccagtgaggatttcgtcattttgagagaaaaaaatattgggtcaatgtcagcgaatgtggcgctctacagcaaggacgaccacctgaacgtaaccgacaagtataacgcgcaagcactcttcaatctggaaatgcacgtactttctcataaatgcaacgatgcgctgaacattgcccttgaagctgttaaagaatctgacttcagtaataaaaaaaaggtcatagatatccttaagaggaagattaatggaatgaaaactacgttctcagaaaaaggatatgctatactgatgaaatacgttaaagcccatcttaatagcaagcattacgcccataacataatttatgggtacgaaaactatttgaagctgcaagagcaattggagcttgcagaaaacgattttaaaacattggagaatattttggtgagaataagaaacaaaatctttaacaaaaaaaacttgatggtcagcgtgacgtccgactatggtgctctcaaacacctgtttgtgaatagcaacgaatccttgaaaaaccttgtaagttacttcgaggaaaacgataagtatatcaacgatatgcagaataaagtgaatgaccctaccgtcatggggtggaatgaagaaatcaagtctaaaaagctgttcgacgaggagaaggtgaaaaaggagttctttgtcttgccgacctttgtcaacagcgtctcaatgagtggtatcctgttcaagccgggcgaatatctcgacccgagtttcactgtcatcgttgcggccctgaaaaatagctatttgtgggacacagttcgaggacttaacggggcttacggtgtattcgctgatatcgaatacgatggctctgtagtatttctttcagctcgagaccccaatctcgaaaagaccctcgccactttccgagaatctgctaaaggattgcgcaaaatggctgacaccatgacagagaacgatttgcttcggtatattattaacacaattggcacgatcgacaagcctcgacgagggatagaactttctaagctgtcattccttagacttatttccaacgagtcagagcaagatcgggtggagttccggaaacggataatgaacacaaaaaaggaagatttctataaattcgcagatcttcttgagagcaaagtaaatgaattcgaaaaaaatatcgttatcataacaacaaaagaaaaggccaacgaatatatagcaaacgtagatggcgagtttaagaaagtcttgatcgagtga

SEQ ID NO: 41 - protein, 1194 aa, unidentified
  REGION 1..1194: note = misc_feature - PfFalcilysin
  source 1..1194: note = Plasmodium falciparum
MANLTKLMKVIGYINIITNCVQSFTNRADKKRYNVFAKSFINTINTNLYTFKAVMSKTPEWIHEKSPKHNSYDIIEKRYNEEFKMTYTVYQHKKAKTQVISLGTNDPLDVEQAFAFYVKTLTHSGKGIPHILEHSVLSGSKNYNYKNSIGLLEKGTLHTHLNAYTFNDRTVYMAGSMNNKDFFNIMGVYMDSVFQPNVLENKYIFETEGWTYEVEKLKEDEKGKAEIPQMKDYKVSFNGIVYNEMKGALSSPLEDLYHEEMKYMFPDNVHSNNSGGDPKEITNLTYEEFKEFYYKNYNPKKVKVFFFSKNNPTELLNFVDQYLGQLDYSKYRDDAVESVEYQTYKKGPFYIKKKYGDHSEEKENLVSVAWLLNPKVDKTNNHNNNHSNNQSSENNGYSNGSHSSDLSLENPTDYFVLLIINNLLIHTPESVLYKALTDCGLGNNVIDRGLNDSLVQYIFSIGLKGIKRNNEKIKNFDKVHYEVEDVIMNALKKVVKEGFNKSAVEASINNIEFILKEANLKTSKSIDFVFEMTSKLNYNRDPLLIFEFEKYLNIVKNKIKNEPMYLEKFVEKHFINNAHRSVILLEGDENYAQEQENLEKQELKKRIENFNEQEKEQVIKNFEELSKYKNAEESPEHLNKFPIISISDLNKKTLEVPVNVYFTNINENNNIMETYNKLKTNEHMLKDNMDVFLKKYVLKNDKHNTNNNNNNNNNMDYSFTETKYEGNVPILVYEMPTTGIVYLQFVFSLDHLTVDELAYLNLFKTLILENKTNKRSSEDFVILREKNIGSMSANVALYSKDDHLNVTDKYNAQALFNLEMHVLSHKCNDALNIALEAVKESDFSNKKKVIDILKRKINGMKTTFSEKGYAILMKYVKAHLNSKHYAHNIIYGYENYLKLQEQLELAENDFKTLENILVRIRNKIFNKKNLMVSVTSDYGALKHLFVNSNESLKNLVSYFEENDKYINDMQNKVNDPTVMGWNEEIKSKKLFDEEKVKKEFFVLPTFVNSVSMSGILFKPGEYLDPSFTVIVAALKNSYLWDTVRGLNGAYGVFADIEYDGSVVFLSARDPNLEKTLATFRESAKGLRKMADTMTENDLLRYIINTIGTIDKPRRGIELSKLSFLRLISNESEQDRVEFRKRIMNTKKEDFYKFADLLESKVNEFEKNIVIITTKEKANEYIANVDGEFKKVLIE

SEQ ID NO: 42 - protein, 1193 aa, unidentified
  REGION 1..1193: note = misc_feature - Falcilysin
  source 1..1193: note = Plasmodium falciparum
MNLTKLMKVIGYINIITNCVQSFTNRADKKRYNVFAKSFINTINTNLYTFKAVMSKTPEWIHEKSPKHNSYDIIEKRYNEEFKMTYTVYQHKKAKTQVISLGTNDPLDVEQAFAFYVKTLTHSGKGIPHILEHSVLSGSKNYNYKNSIGLLEKGTLHTHLNAYTFNDRTVYMAGSMNNKDFFNIMGVYMDSVFQPNVLENKYIFETEGWTYEVEKLKEDEKGKAEIPQMKDYKVSFNGIVYNEMKGALSSPLEDLYHEEMKYMFPDNVHSNNSGGDPKEITNLTYEEFKEFYYKNYNPKKVKVFFFSKNNPTELLNFVDQYLGQLDYSKYRDDAVESVEYQTYKKGPFYIKKKYGDHSEEKENLVSVAWLLNPKVDKTNNHNNNHSNNQSSENNGYSNGSHSSDLSLENPTDYFVLLIINNLLIHTPESVLYKALTDCGLGNNVIDRGLNDSLVQYIFSIGLKGIKRNNEKIKNFDKVHYEVEDVIMNALKKVVKEGFNKSAVEASINNIEFILKEANLKTSKSIDFVFEMTSKLNYNRDPLLIFEFEKYLNIVKNKIKNEPMYLEKFVEKHFINNAHRSVILLEGDENYAQEQENLEKQELKKRIENFNEQEKEQVIKNFEELSKYKNAEESPEHLNKFPIISISDLNKKTLEVPVNVYFTNINENNNIMETYNKLKTNEHMLKDNMDVFLKKYVLKNDKHNTNNNNNNNNNMDYSFTETKYEGNVPILVYEMPTTGIVYLQFVFSLDHLTVDELAYLNLFKTLILENKTNKRSSEDFVILREKNIGSMSANVALYSKDDHLNVTDKYNAQALFNLEMHVLSHKCNDALNIALEAVKESDFSNKKKVIDILKRKINGMKTTFSEKGYAILMKYVKAHLNSKHYAHNIIYGYENYLKLQEQLELAENDFKTLENILVRIRNKIFNKKNLMVSVTSDYGALKHLFVNSNESLKNLVSYFEENDKYINDMQNKVNDPTVMGWNEEIKSKKLFDEEKVKKEFFVLPTFVNSVSMSGILFKPGEYLDPSFTVIVAALKNSYLWDTVRGLNGAYGVFADIEYDGSVVFLSARDPNLEKTLATFRESAKGLRKMADTMTENDLLRYIINTIGTIDKPRRGIELSKLSFLRLISNESEQDRVEFRKRIMNTKKEDFYKFADLLESKVNEFEKNIVIITTKEKANEYIANVDGEFKKVLIE

SEQ ID NO: 43 - DNA, 3471 bp, synthetic construct
  misc_feature 1..3471: note = PvFalcilysin Human CO
  source 1..3471: mol_type = other DNA
gccaccatggctaaactgatgagggtctttggttatcttaacataattactaactgcgttaacggacttctctgtaaagcggagaaacgcaaatataacgtctttacgaacagctttatctattctatctctacaacaaatctgtactccttcacggccatgatgaacaaatcacccgagtgggtgcaggaaaagtgtcccgagcacaagtcttataacatccttgaaaagcggttttctgataaattccaaatgacgtatacagtgtacgagcacaaaaaagcgaaaacccaagtcattgccctcggcagcaatgaccctctggacgttgagcagacttttgccttttacgtaaagaccctgacgaactcaggtaagggtattcctcacatactcgagcacacagttttgagcgggagcaaaaatttcaattacaaagatagcatgggactcctggaaaagggaacacttaacacccatttgaatgcgtatactttcaacgacaggactgtgtatatggcggggtctatgaataatagagactttttcaacataatggcggtgtatatggatagcgtgtttcaaccgaatgttcttgaaaataagtttatcttccaaacggaaggatggacctacgaagtggaaaagttgaaagatgaggagaagaacgctgatgtaccgaaaattaaagattataaggtttcctacaatggtatagtctattccgaaatgaaagggagcttctcctctcccctgcaatatctttattatttgatcatgaaaaacatcttccctgacaacgtccattctaacataagtggaggggaccctaaggagattccaacgctcacgtatgaggaattcaaagagttctactataagaattacaatcctaaaaaaattaaagtcattttcttcagcaaaaacaatcctacagagttgttgaatttcgtcgacgattatttgaaccagctcgactttactaaatacagagacgatgcagtagaaaacgttaattatcaggagtacaagaaggggccattctatgttaagaagaaatttgcggatcacagtgaggaaaaggaaaatcttgtttccatttcatggcttctcaacccgaaaaaaaatgacctcctggatgttgatcttagccttgaaagcccccaagactacttcgctttgttgattattaacaaccttctgacgcacaccaccgaatctgtgctttacaaggcgcttattgactgcggtttgggtaacaccgttatcgatacaggccttgacgacagcctggtacagttcatattttctataggtctgaagggtatcaaggaaaagaatgaaaagaacgtatcattggacgttgtccattatgaagtggaaaaggtggttctcaaagcgctccaaaaagtagtggatgaaggcttcaacaaatcagcggtcgaggccagcattaacaacatagaattcgttttgaaagaagccaatcttaaaacgtccaaaagcgtggactacatatttgaaatggcttccaggctgaattataatagagaccctctgctgatctttgaattcgagaagcacttgaacgtcgttaaagataagatcaagaatgaaccaaagtatttggagaaattcattgagaaacactttataaacaataaccatcgggctgtcatcttgatggagggtgatgaaaactatgggaaagaacaggaggatttggaaaaggagactttgaaaaagaagatcgaatcactcacggaaaaagaacgggacgacataatagttgatttcgagaacctgacaaagtataagaacatggtcgagagccccgaacatttggataattttcccatcatctctatcagcgatctgaacaaagaaacgctggaaatccctgctaatgcatatttcacgtccacggcagaggaaaataatatggaaaagtacaataaagtgaaggcaagcgaggatgttatgaagaaaaacatggaccacctcatcgacaaatatgttctgaaaggagcgcaaggaggcgctgctaccgacggtgcagccaaacagggtgattccagcgatggtgaaatccccatgctcgtttacgagatgcctaccagtggaatcttgtatctgcaatttatctttaacctcgatcatctgagcctggaggaaatgtcatatctcaatctctttaagatgctcatccttgagaacaaaactatgaaacgctcctctgaggagtttgtcattttgagggaaaagaatatcggcaacataatggctaacgtcgccttgtatagtatcagcgatcacctcaaggtcacttctaaatacaacgctcacggtctgttcaattttgaaatgcatgtattgagtcacaagtgcaatgagtctttggaaattgccttggaagctctcaaggactctgattttagcaacaagaagaaaatagtggagatcctgaaacggaagatcaacggcatgaagacggtcttctcttctaaaggctactcacttctcctcaagtatgtaaagtcacaaatgaatgcaaaatactacgctcatgatttggtttttggatacggcaactatttgaagttgcaagaacagctcaagctcgccgaaagtgactttccacagttcgagcagattctcaacagaatccgaaataagattttcactaagaagaatctgctgatcagcgtgaccagtgacgctgcagcgttggatcaactgtttgtgcatagcaaggaatccctgaagaaccttcttgggtatttcgaagagaatgatgccaagtctggagaggctgagaccatagggtggaatgaggagattaaacaatcaaaagtgatcgaaaaggaacaaaagaagaaggaattctttgtaataccaacatttgtcaatgcagtatcaatggctggaatgttgtttaatgagaaggagttcctggatccgtctttcatagttatcgtggccgcattgaaaaactcatacctttgggagaccgtgaggggactcaacggggcatatggagtttttgctgatatagaatacgatggtgccgtcgtgttcttgtcagcccgcgatccgaatctggaaaagacgctgcaaacgttcaaggagtctgctcaagggttgagaaagatggcggatacaatgaccaaaaatgaccttcggcgctacataatcaatgcgatcggcaatattgataagccgagacggggtgttgagctctcaaagctctcacttttgagaattatatctaatgaaaccaaacaggaccgaatcgactttcgcaagaggattatggagacgacgaaggaagacttctataaattcgctgacttgcttgaaaagaagattgcagaatttgaaaagaatattgttattatcacatctaaggaaaaagcctcagaatacagcactaacgtagaccaggattttaagcagattcatatcgagtag

SEQ ID NO: 44 - protein, 1154 aa, unidentified
  REGION 1..1154: note = misc_feature - PvFalcilysin
  source 1..1154: note = Plasmodium vivax
MAKLMRVFGYLNIITNCVNGLLCKAEKRKYNVFTNSFIYSISTTNLYSFTAMMNKSPEWVQEKCPEHKSYNILEKRFSDKFQMTYTVYEHKKAKTQVIALGSNDPLDVEQTFAFYVKTLTNSGKGIPHILEHTVLSGSKNFNYKDSMGLLEKGTLNTHLNAYTFNDRTVYMAGSMNNRDFFNIMAVYMDSVFQPNVLENKFIFQTEGWTYEVEKLKDEEKNADVPKIKDYKVSYNGIVYSEMKGSFSSPLQYLYYLIMKNIFPDNVHSNISGGDPKEIPTLTYEEFKEFYYKNYNPKKIKVIFFSKNNPTELLNFVDDYLNQLDFTKYRDDAVENVNYQEYKKGPFYVKKKFADHSEEKENLVSISWLLNPKKNDLLDVDLSLESPQDYFALLIINNLLTHTTESVLYKALIDCGLGNTVIDTGLDDSLVQFIFSIGLKGIKEKNEKNVSLDVVHYEVEKVVLKALQKVVDEGFNKSAVEASINNIEFVLKEANLKTSKSVDYIFEMASRLNYNRDPLLIFEFEKHLNVVKDKIKNEPKYLEKFIEKHFINNNHRAVILMEGDENYGKEQEDLEKETLKKKIESLTEKERDDIIVDFENLTKYKNMVESPEHLDNFPIISISDLNKETLEIPANAYFTSTAEENNMEKYNKVKASEDVMKKNMDHLIDKYVLKGAQGGAATDGAAKQGDSSDGEIPMLVYEMPTSGILYLQFIFNLDHLSLEEMSYLNLFKMLILENKTMKRSSEEFVILREKNIGNIMANVALYSISDHLKVTSKYNAHGLFNFEMHVLSHKCNESLEIALEALKDSDFSNKKKIVEILKRKINGMKTVFSSKGYSLLLKYVKSQMNAKYYAHDLVFGYGNYLKLQEQLKLAESDFPQFEQILNRIRNKIFTKKNLLISVTSDAAALDQLFVHSKESLKNLLGYFEENDAKSGEAETIGWNEEIKQSKVIEKEQKKKEFFVIPTFVNAVSMAGMLFNEKEFLDPSFIVIVAALKNSYLWETVRGLNGAYGVFADIEYDGAVVFLSARDPNLEKTLQTFKESAQGLRKMADTMTKNDLRRYIINAIGNIDKPRRGVELSKLSLLRIISNETKQDRIDFRKRIMETTKEDFYKFADLLEKKIAEFEKNIVIITSKEKASEYSTNVDQDFKQIHIE

SEQ ID NO: 45 - protein, 1153 aa, unidentified
  REGION 1..1153: note = misc_feature - Falcilysin
  source 1..1153: note = Plasmodium vivax
MKLMRVFGYLNIITNCVNGLLCKAEKRKYNVFTNSFIYSISTTNLYSFTAMMNKSPEWVQEKCPEHKSYNILEKRFSDKFQMTYTVYEHKKAKTQVIALGSNDPLDVEQTFAFYVKTLTNSGKGIPHILEHTVLSGSKNFNYKDSMGLLEKGTLNTHLNAYTFNDRTVYMAGSMNNRDFFNIMAVYMDSVFQPNVLENKFIFQTEGWTYEVEKLKDEEKNADVPKIKDYKVSYNGIVYSEMKGSFSSPLQYLYYLIMKNIFPDNVHSNISGGDPKEIPTLTYEEFKEFYYKNYNPKKIKVIFFSKNNPTELLNFVDDYLNQLDFTKYRDDAVENVNYQEYKKGPFYVKKKFADHSEEKENLVSISWLLNPKKNDLLDVDLSLESPQDYFALLIINNLLTHTTESVLYKALIDCGLGNTVIDTGLDDSLVQFIFSIGLKGIKEKNEKNVSLDVVHYEVEKVVLKALQKVVDEGFNKSAVEASINNIEFVLKEANLKTSKSVDYIFEMASRLNYNRDPLLIFEFEKHLNVVKDKIKNEPKYLEKFIEKHFINNNHRAVILMEGDENYGKEQEDLEKETLKKKIESLTEKERDDIIVDFENLTKYKNMVESPEHLDNFPIISISDLNKETLEIPANAYFTSTAEENNMEKYNKVKASEDVMKKNMDHLIDKYVLKGAQGGAATDGAAKQGDSSDGEIPMLVYEMPTSGILYLQFIFNLDHLSLEEMSYLNLFKMLILENKTMKRSSEEFVILREKNIGNIMANVALYSISDHLKVTSKYNAHGLFNFEMHVLSHKCNESLEIALEALKDSDFSNKKKIVEILKRKINGMKTVFSSKGYSLLLKYVKSQMNAKYYAHDLVFGYGNYLKLQEQLKLAESDFPQFEQILNRIRNKIFTKKNLLISVTSDAAALDQLFVHSKESLKNLLGYFEENDAKSGEAETIGWNEEIKQSKVIEKEQKKKEFFVIPTFVNAVSMAGMLFNEKEFLDPSFIVIVAALKNSYLWETVRGLNGAYGVFADIEYDGAVVFLSARDPNLEKTLQTFKESAQGLRKMADTMTKNDLRRYIINAIGNIDKPRRGVELSKLSLLRIISNETKQDRIDFRKRIMETTKEDFYKFADLLEKKIAEFEKNIVIITSKEKASEYSTNVDQDFKQIHIE

SEQ ID NO: 46 - DNA, 1227 bp, synthetic construct
  misc_feature 1..1227: note = Falcilysin, Bergheilysin-B-CO
  source 1..1227: mol_type = other DNA
ggatccgccaccatggctaaactcatgaaggtcttggggtatatcaacatcatcacgaattgcgtgaatggcatactgtgtaaaggcgataagaagcgatacatgcacatgctctcccataagtgtaacgacgctctggagattgcgctggaagccctgaaagaaagcgatttcagcaataagaagaaagtgattgaaatcctgaagaggaaaatcaatggcatgaaaaccacgtttgccagcaaaggtcactcaatactgattaagtacgtgaagtctcggattaatagcaaatattacgcttatgacctgattcatgggtatgacaactatctgaagctccaggaacagcttaaactggcggagacaaactacgagagtctggaagccatacttaatcgcatcaggaagaagatctttaagcggaacaatctgatcatgaacgtgactgtcgatccaggaactattgatcagctgtttgccaaatccaagaatagcttcaacaaccttctgtcatactttgacgagaacgagagctactgctctaagaatgactccttcaacaaagtggtaggctggaacaaggagattcaggagaagaagctgcttgaaggagaggaggtcaagaaagagctcttggtcgtacccacatttgtgaacagtgtctctatgagtggagtgctgttcaacaaaggcgaatatcttgacccctcattcaccgtaatcgttgcagccttgaaaaactcatacctttgggagacagttcgaggactgaatggagcttatggggtgtttgccgacatagagtatgacggtactgtcgttttcctgagtgcaagagatccgaatctggaaaagactctccaaacctttcgtgaagcagcacaaggtctgcgtaaaatggctgatgtgatgacaaagaatgatcttctgcggtatatcattaacgctattgggaccatcgatagacctcgcagaggcgttgagttgtccaagctgtccttctctcgcataataagcaacgaaaccgaacaggacaggattgaattcaggaaccgggtgatgaataccaagaaagaggatttctacaaatttgcggatttgctcgaaaagaaagtcaaggaatttgagaagaatgtggtgatcatcacatctaaggagaaagccaacgagtacatcaacaatgttgacaatgacttcaagaagatcctcattgagtaactcgag

SEQ ID NO: 47 - protein, 402 aa, unidentified
  REGION 1..402: note = misc_feature - Falcilysin, Bergheilysin-B
  source 1..402: note = Plasmodium berghei
MAKLMKVLGYINIITNCVNGILCKGDKKRYMHMLSHKCNDALEIALEALKESDFSNKKKVIEILKRKINGMKTTFASKGHSILIKYVKSRINSKYYAYDLIHGYDNYLKLQEQLKLAETNYESLEAILNRIRKKIFKRNNLIMNVTVDPGTIDQLFAKSKNSFNNLLSYFDENESYCSKNDSFNKVVGWNKEIQEKKLLEGEEVKKELLVVPTFVNSVSMSGVLFNKGEYLDPSFTVIVAALKNSYLWETVRGLNGAYGVFADIEYDGTVVFLSARDPNLEKTLQTFREAAQGLRKMADVMTKNDLLRYIINAIGTIDRPRRGVELSKLSFSRIISNETEQDRIEFRNRVMNTKKEDFYKFADLLEKKVKEFEKNVVIITSKEKANEYINNVDNDFKKILIE

SEQ ID NO: 48 - DNA, 2454 bp, unidentified
  misc_feature 1..2454: note = perforin like protein 1 (SPECT2)
  source 1..2454: mol_type = unassigned DNA; note = Plasmodium berghei
ggatccgccaccatggctaagatgcggaacattaagaaatcacttccggtgctcttcattctcttgtgcatttatcagcagtactttataaactctctccgtatttccgtccgcaacaataagaaccacagggatgaaaacaaattcaacaagaatatggagcttggtacgatggagaaacccatcaacattctttgcaatgacgtcagctgtaacacagagaataacatttctttcgtaaaccagaagaaaaaggaaatagatagtgacagcgacctctataacatgttcgatgacgacgcctctacctccgctggtgatgacgaagatgactatgacgactacacagatgataagaacgctgagatcaaagatgaagagcaaaatgagcacatcgacaagatcgaccagaagaaggataagaaacgcacattctctatcaataagcaggaggaagaaatcaacgaaaataaaaacaaaacggagaaattcttcaagaaatacaagtttaacgacgccaactcagaaggggacgacgatgagtcagacaccgatgacgagaatttggacaactccacggagaacagctacgaggaaaacaagaatcccgagaacgttatcgacaagcatatggccgtatttcctgggctctattttgtggggatcggctacgatattctgttcgggaatcctctcggagaaaccgattctctgagcgaccctggttatagagcacagatttacctgttgaattgggaattctcaaaccatggcatcgccaacgatctgcacacgctccaaccaattaatgcctggattcggaaagagaatgcatgctcacgagttgaatccatcaacgaatgttcctctgttagtgagtatacaaagaatctgtcagtggacgtaagtgtatcaggcagctatatgggcttcggcagtttctccgctagcactgggtacaagaaattcattaacgagataagcaagagaacatccaagacctactttataaagagcaactgcatcaagtataccatcggacttccaccttatgtgccatgggagcataccactgcttacatgaatgcggtgaatattctgccaaaggaatttaccggcctggatggagacagcgaatgcacaccggatgtttacgagcagaagaagatgactaaacagtgtaagaacgtgcaactgtggattcagttctttaagacctatggtacacacataatcgtggaggctcaattgggagggaaaataaccaaaatcatcaatgtctctaacacaagcgtgaaccagatgaagaaggatggcgtcagtgtgaaagcccagatccaggctcagtttggttttgcaagtgtgggcggtagtacaagcgtgtcaagtgacaatagcactaagaacgacaatagcagctacgacatgtctgagaaactcgtggttatcggagggaatcctataaaagacgtcaccaaagaggagaatctgtacgaatggagcaaaacagtgtcctctaaccccatgcctatccacatcaagctgctgccaatctataagtccttcgatagtgaggaactgaaagagtcttacgagaaagcggttctctactacaccaggctttatggcagctctccccacggaactattcagaaagatgagaacgacatcatcaaaatcttgacggccagtaccaccatcactaaaattggtgctccacccataacagcggaatgtccacataatcaagtggtgctgtttgggtatgtcctgaagcagaacttctgggacaacacctccaacctgaagggatacgacattgagatctgtgaggctggactgaatagttgcacgtccaaacagggaagtacaaacaagtacgatgtgagctatctgtacattgaatgtggcacacaggcaatgtcattctccgatcaagtcataaccgcatccaacactacttacaataccatcaagtgtcccaatgactacactattatctttggattcgggttttcctctagctccggtaagggagtttctgccatgcacacccacattacatcctgtagacccggcatgaaaagctgctccctgaatatgggcaacagcaatgacaagaactacatgtacctggtgtgcgtcgatgccacaatctggtctggcattaatgagctgactattgtggccaaagatgattttcacggcgcagtgaataggtctaagcagttcaatgatggcgaattggtactgtcctgtcaggaaaatggcactatcctgacagggttcactggggagactcatacctctagcccgtatgtcaagagcccttttagcaagtgtcttaaatcactgaagagctgctcagtccatgggagtggacaatctatcggatataccaactataagtcactgttttccatcatactgtgcaagaacggtgagtgactcgag

SEQ ID NO: 49 - protein, 811 aa, unidentified
  REGION 1..811: note = misc_feature - perforin like protein 1 (SPECT2)
  source 1..811: note = Plasmodium berghei
MAKMRNIKKSLPVLFILLCIYQQYFINSLRISVRNNKNHRDENKFNKNMELGTMEKPINILCNDVSCNTENNISFVNQKKKEIDSDSDLYNMFDDDASTSAGDDEDDYDDYTDDKNAEIKDEEQNEHIDKIDQKKDKKRTFSINKQEEEINENKNKTEKFFKKYKFNDANSEGDDDESDTDDENLDNSTENSYEENKNPENVIDKHMAVFPGLYFVGIGYDILFGNPLGETDSLSDPGYRAQIYLLNWEFSNHGIANDLHTLQPINAWIRKENACSRVESINECSSVSEYTKNLSVDVSVSGSYMGFGSFSASTGYKKFINEISKRTSKTYFIKSNCIKYTIGLPPYVPWEHTTAYMNAVNILPKEFTGLDGDSECTPDVYEQKKMTKQCKNVQLWIQFFKTYGTHIIVEAQLGGKITKIINVSNTSVNQMKKDGVSVKAQIQAQFGFASVGGSTSVSSDNSTKNDNSSYDMSEKLVVIGGNPIKDVTKEENLYEWSKTVSSNPMPIHIKLLPIYKSFDSEELKESYEKAVLYYTRLYGSSPHGTIQKDENDIIKILTASTTITKIGAPPITAECPHNQVVLFGYVLKQNFWDNTSNLKGYDIEICEAGLNSCTSKQGSTNKYDVSYLYIECGTQAMSFSDQVITASNTTYNTIKCPNDYTIIFGFGFSSSSGKGVSAMHTHITSCRPGMKSCSLNMGNSNDKNYMYLVCVDATIWSGINELTIVAKDDFHGAVNRSKQFNDGELVLSCQENGTILTGFTGETHTSSPYVKSPFSKCLKSLKSCSVHGSGQSIGYTNYKSLFSIILCKNGE

SEQ ID NO: 50 - protein, 810 aa, unidentified
  REGION 1..810: note = misc_feature - SPECT2
  source 1..810: note = Plasmodium berghei
MKMRNIKKSLPVLFILLCIYQQYFINSLRISVRNNKNHRDENKFNKNMELGTMEKPINILCNDVSCNTENNISFVNQKKKEIDSDSDLYNMFDDDASTSAGDDEDDYDDYTDDKNAEIKDEEQNEHIDKIDQKKDKKRTFSINKQEEEINENKNKTEKFFKKYKFNDANSEGDDDESDTDDENLDNSTENSYEENKNPENVIDKHMAVFPGLYFVGIGYDILFGNPLGETDSLSDPGYRAQIYLLNWEFSNHGIANDLHTLQPINAWIRKENACSRVESINECSSVSEYTKNLSVDVSVSGSYMGFGSFSASTGYKKFINEISKRTSKTYFIKSNCIKYTIGLPPYVPWEHTTAYMNAVNILPKEFTGLDGDSECTPDVYEQKKMTKQCKNVQLWIQFFKTYGTHIIVEAQLGGKITKIINVSNTSVNQMKKDGVSVKAQIQAQFGFASVGGSTSVSSDNSTKNDNSSYDMSEKLVVIGGNPIKDVTKEENLYEWSKTVSSNPMPIHIKLLPIYKSFDSEELKESYEKAVLYYTRLYGSSPHGTIQKDENDIIKILTASTTITKIGAPPITAECPHNQVVLFGYVLKQNFWDNTSNLKGYDIEICEAGLNSCTSKQGSTNKYDVSYLYIECGTQAMSFSDQVITASNTTYNTIKCPNDYTIIFGFGFSSSSGKGVSAMHTHITSCRPGMKSCSLNMGNSNDKNYMYLVCVDATIWSGINELTIVAKDDFHGAVNRSKQFNDGELVLSCQENGTILTGFTGETHTSSPYVKSPFSKCLKSLKSCSVHGSGQSIGYTNYKSLFSIILCKNGE

SEQ ID NO: 51 - DNA, 2538 bp, synthetic construct
  misc_feature 1..2538: note = PfSPECT2 human CO
  source 1..2538: mol_type = other DNA
gccaccatggctaagctgagaatcctcaagaagcactattatgtcgtgtttatccttctgtatctgtatgacatcagctgcttcaagtgcattcggctgaacaaccgttccatctacaagaacaagtacaagaataacgtgcacataggaaccaatgagaatatcaggagtattgagaagtatagcaacgtgctgtgtaacagcatcttgtgtaagaacgataaaatctctagcttcattaaccagaggaagaatgtggatgacgacgatgagtctgagaacgatgacatgtacgagagcaccacagctggctcttctagtgaaacggacaacgagagcgatgaagaagagaatgacagcagtgacaacaataatagcgatgaggaacagatagagaactccaacaacaataattctgacgaggaacagaatgactcctcttccaacgacaataatgatgaggagaatgaggaacaggacgacgtcatggacaatgaccaaaacgataagaagatcaagcatagcttcaatctcgccaacgagagtaaacacactaaagaggaacgagtgaaagaagagaaaaagctgaagatctatgacttcataaacgacaaggagaaaagacttaactttaatggcgatcagaaagatgaagataacgaggagaacgatgataaagatgagaacacgcttgagaatcggaatatcatctccaaacacacttcagtgtttcctggcctgtacttcatcgggattgggtataacctcctcttcgggaaccccttgggtgaagctgattcccttatcgatccaggttatcgggcgcaaatttacctgatggaatgggctctcagcaaggaaggcattgccaacgacctgagcactctgcaacccgtgaatggatggatacgaaaggagaatgcctgctccagagttgaatctattacagaatgcagctctatatctgactacaccaaatccctgtcagcggaggcaaaggttagtggctcttattggggaatcgcctccttctcagcatccaccgggtatagctcttttctccacgaggtgacaaaacgcagcaagaaaaccttcctcgtgaaatccaactgtgtgaaatacactatcgggcttcctccctatattccctgggacaagaccacggcctacaagaatgccgtaaatgaactgccagctgtatttaccggtttggataaagaatccgaatgtccctctgatgtgtacgaagagaacaagacaaaatcaaactgcgagaacgtgagtctgtggatgaagttcttcgacatttatggcactcacatcatttatgaaagtcaattgggaggaaagataacaaagattatcaatgtttccacctcaagtattgagcagatgaagaaaaatggagtctcagtcaaagcgaaaattcaagcacagtttgggtttggttcagccggtggctcaaccgacgtgaatagcagtaactcctccgcaaatgatgagcagtcttacgacatgaatgaacagctgatcgtcataggagggaatccgatcaaagacgtcaccaaggaggagaatctgtttgagtggtctaaaactgtgacaaaccatcctatgccgatcaacattaaactgactcccattagcgacagttttgactcagacgacctgaaagaatcctatgataaagccatcatctactattctcgcctgtacggactgtcccctcatgacacaatgcagaaggatgacaaggatattatcaagatcctgaccaacgctgatacggttaccaagaactcagctcctccaatcaacgctcagtgtcctcatgggaaagtcgttatgttcggattcagcctgaagcagaatttctgggacaacaccaacgcactcaaaggatacaacatcgaagtctgcgaagcagggagcaattcttgcactagcaaacaagggagcagcaataaatacgatacatcttacctttacatggaatgtggcgatcagccattgccctttagcgagcaggtgattagcgagagtacaagtacctataataccgtgaaatgtccgaatgattactccattctcctgggcttcggaatatcaagtagctcagggaggataaatagcgctgaatatgtctacagcacaccatgtattcccggcatgaagtcctgcagcctcaatatgaataatgacaaccagaagtcatacatctacgtgctgtgtgtagatactacgatctggagtggcgtgaacaacctgagcctggttgcccttgatggcgcacatggtaaggtaaaccgcagtaagaagtactccgacggtgaactggttggcacctgtccactggacggcacagtcctgactggatttaaggttgagtttcacacttcatctccatatgtgcagacacctttcgagaaatgcgccaaaagcttgaaagcctgctccgtacatggctccggtcacgccattggcattcagaactttaagtcactgttcatatacatgttgtgcaagaacaataagtaa

SEQ ID NO: 52 - protein, 843 aa, unidentified
  REGION 1..843: note = misc_feature - PfSPECT2
  source 1..843: note = Plasmodium falciparum
MAKLRILKKHYYVVFILLYLYDISCFKCIRLNNRSIYKNKYKNNVHIGTNENIRSIEKYSNVLCNSILCKNDKISSFINQRKNVDDDDESENDDMYESTTAGSSSETDNESDEEENDSSDNNNSDEEQIENSNNNNSDEEQNDSSSNDNNDEENEEQDDVMDNDQNDKKIKHSFNLANESKHTKEERVKEEKKLKIYDFINDKEKRLNFNGDQKDEDNEENDDKDENTLENRNIISKHTSVFPGLYFIGIGYNLLFGNPLGEADSLIDPGYRAQIYLMEWALSKEGIANDLSTLQPVNGWIRKENACSRVESITECSSISDYTKSLSAEAKVSGSYWGIASFSASTGYSSFLHEVTKRSKKTFLVKSNCVKYTIGLPPYIPWDKTTAYKNAVNELPAVFTGLDKESECPSDVYEENKTKSNCENVSLWMKFFDIYGTHIIYESQLGGKITKIINVSTSSIEQMKKNGVSVKAKIQAQFGFGSAGGSTDVNSSNSSANDEQSYDMNEQLIVIGGNPIKDVTKEENLFEWSKTVTNHPMPINIKLTPISDSFDSDDLKESYDKAIIYYSRLYGLSPHDTMQKDDKDIIKILTNADTVTKNSAPPINAQCPHGKVVMFGFSLKQNFWDNTNALKGYNIEVCEAGSNSCTSKQGSSNKYDTSYLYMECGDQPLPFSEQVISESTSTYNTVKCPNDYSILLGFGISSSSGRINSAEYVYSTPCIPGMKSCSLNMNNDNQKSYIYVLCVDTTIWSGVNNLSLVALDGAHGKVNRSKKYSDGELVGTCPLDGTVLTGFKVEFHTSSPYVQTPFEKCAKSLKACSVHGSGHAIGIQNFKSLFIYMLCKNNK

SEQ ID NO: 53 - protein, 842 aa, unidentified
  REGION 1..842: note = misc_feature - SPECT2
  source 1..842: note = Plasmodium falciparum
MKLRILKKHYYVVFILLYLYDISCFKCIRLNNRSIYKNKYKNNVHIGTNENIRSIEKYSNVLCNSILCKNDKISSFINQRKNVDDDDESENDDMYESTTAGSSSETDNESDEEENDSSDNNNSDEEQIENSNNNNSDEEQNDSSSNDNNDEENEEQDDVMDNDQNDKKIKHSFNLANESKHTKEERVKEEKKLKIYDFINDKEKRLNFNGDQKDEDNEENDDKDENTLENRNIISKHTSVFPGLYFIGIGYNLLFGNPLGEADSLIDPGYRAQIYLMEWALSKEGIANDLSTLQPVNGWIRKENACSRVESITECSSISDYTKSLSAEAKVSGSYWGIASFSASTGYSSFLHEVTKRSKKTFLVKSNCVKYTIGLPPYIPWDKTTAYKNAVNELPAVFTGLDKESECPSDVYEENKTKSNCENVSLWMKFFDIYGTHIIYESQLGGKITKIINVSTSSIEQMKKNGVSVKAKIQAQFGFGSAGGSTDVNSSNSSANDEQSYDMNEQLIVIGGNPIKDVTKEENLFEWSKTVTNHPMPINIKLTPISDSFDSDDLKESYDKAIIYYSRLYGLSPHDTMQKDDKDIIKILTNADTVTKNSAPPINAQCPHGKVVMFGFSLKQNFWDNTNALKGYNIEVCEAGSNSCTSKQGSSNKYDTSYLYMECGDQPLPFSEQVISESTSTYNTVKCPNDYSILLGFGISSSSGRINSAEYVYSTPCIPGMKSCSLNMNNDNQKSYIYVLCVDTTIWSGVNNLSLVALDGAHGKVNRSKKYSDGELVGTCPLDGTVLTGFKVEFHTSSPYVQTPFEKCAKSLKACSVHGSGHAIGIQNFKSLFIYMLCKNNK

SEQ ID NO: 54 - DNA, 2541 bp, synthetic construct
  misc_feature 1..2541: note = Synthetic sequence
  misc_feature 1..2541: note = PvSPECT2 human CO
  source 1..2541: mol_type = other DNA
gccaccatggctaagccaagaaatataaactctctgttggccatatggtgtattctcttctcaatctgtgagtacggctatgtgggatctttgagaataggtcttcggagaaactatagtcaaggggactcctctgatcggctcgggggtagtgacgtggacgtgagacggtccggcgagcttatggacctcggtaagaacgcaaacatcctctgcaataacatcagctgtaacagtaaaaaagaggcttcttttctgagtcagaaaaaacaacttgaggatgacgatgacgacgagctggccgcgctgtacaatgatgatgatgacgatgctagcacaaccactggggggggttcagaaactagcgatgacgacgagcttgaactccctgatcaggacgaaggtgcggacgagggtgaagcagaggaccaactcgcgacacaggaggattccgacaactcagggaccgaccaaggtctgaaaaaaaaagtaaaccttagccgccacgaaaagttgatagaagacaaaaaacaacagacagagaatacttttaagaagtacaggtttggggatgaggaggaagagtcagaggagaaatccccggggaaatctaagtcactcgatccaagcagcctcgatgatgacgacggcgaaggtgatgatgatgatgatggcgacgagagagaaaagaagcaaagtaatacaaggaaagccatgaagaaggacttggatgttttcccagggctttactttgcaggcatcggatacgatagcctcttcggaaaccccctgggcgaggctgacagcttgaccgatcccgggtaccggggccaaatcatactcatgaactgggaactctcaaataagggagttgcgaatgatcttgcgacattgcaacctctgaatggatggatccgcaaggaaaatgcgtgcagtcgagcagaatctataaaagagtgttcttcagtatcagactacacgaaaaatttgaccgcagaggcatctgtatctggctcctacatgggctttggagcttttagtgctagtactggttataagaaatttttgcaagaagccagtaaacgcacgtccaaaacttatctggtaaaaagcaattgcgtgaaatacacggtcgggctccctccttatgtgcgctgggagcagacgaccgcctttaagaacgctgtgaacgggttgccaccgcatttcaccggactggaggccgactctgaatgcgcctctgatgtctacgaacaaaagaagacatctgaagaatgcgaaaccgtacatgcctggatacgcttttttaagacttatggaacacacgtgatcatggaagcacagcttggcggcaaaataacaaagattatccgggtcgagaatagctccgtcaatcaaatgaagaaagacggagtcagcgtgaaagcccagatcaaagcccaattcggctttgcatctgtggggggaagcacaaacgtctctagtgaccatagttcaaaaaagaacgaggataattatgagatgtctgagcagctggtagtgattggaggcaacccaatcaaggatgtgacgaaagaagaaaatctctatgagtggtctaaaacggtctcaacaaatccgatgcctatcaatatcaagctcctgccgattagcactatctttgactccgacgatctgaaaaatagttacgaaaaggcattgatctactacactcgcttgtatggcttttcaccccatgatacaatgcaaaaagatgaaaaggatattgttaagatcctgacggccagcaccacagtcacaaagacaggcccgccgcctatatctgcagagtgtccgcataatatggtggtcttgttcggtttcgtagtcaaacagaatttttgggatcatacgaacaaactccagagttatgaaatggagatttgtgaaagcggtgccagctcatgcacgtctaagcagggaaatacaaataagtatgacgtatcctatacgtatattgagtgcggaccacaggcattgccgttcactgagcaggttgtatctgtatccggtacaacatataactctgttaaatgcccgaatgactacagcgtgttgtttggctttggaatggctacgagttctggcaggcaccaaagtgcgctttatagctatttcacaccatgtcgaccagggctcaaaagctgtagcttgaatatgaacgaacatgatgataagtcttacatttacctggtctgcgtcgacgcgactatctggacgggacttaacgcgttgagcatgatcgcgaaagacgatttgcacagtgccgtgaaccggtaccaacaattcaatgatggagagctggttgtgacgtgtcctagcgagggcactatactgactggattctatggggagacccatacctccagtccatatgttactgtaccttttgggaaatgcgcaaagtcattgaaagcctgttctgttcatgggtccggccaagccataggcattcacaattatagaactttgttcacagtcgcactgtgtaagaataattag

SEQ ID NO: 55 - protein, 844 aa, unidentified
  REGION 1..844: note = misc_feature - PvSPECT2
  source 1..844: note = Plasmodium vivax
MAKPRNINSLLAIWCILFSICEYGYVGSLRIGLRRNYSQGDSSDRLGGSDVDVRRSGELMDLGKNANILCNNISCNSKKEASFLSQKKQLEDDDDDELAALYNDDDDDASTTTGGGSETSDDDELELPDQDEGADEGEAEDQLATQEDSDNSGTDQGLKKKVNLSRHEKLIEDKKQQTENTFKKYRFGDEEEESEEKSPGKSKSLDPSSLDDDDGEGDDDDDGDEREKKQSNTRKAMKKDLDVFPGLYFAGIGYDSLFGNPLGEADSLTDPGYRGQIILMNWELSNKGVANDLATLQPLNGWIRKENACSRAESIKECSSVSDYTKNLTAEASVSGSYMGFGAFSASTGYKKFLQEASKRTSKTYLVKSNCVKYTVGLPPYVRWEQTTAFKNAVNGLPPHFTGLEADSECASDVYEQKKTSEECETVHAWIRFFKTYGTHVIMEAQLGGKITKIIRVENSSVNQMKKDGVSVKAQIKAQFGFASVGGSTNVSSDHSSKKNEDNYEMSEQLVVIGGNPIKDVTKEENLYEWSKTVSTNPMPINIKLLPISTIFDSDDLKNSYEKALIYYTRLYGFSPHDTMQKDEKDIVKILTASTTVTKTGPPPISAECPHNMVVLFGFVVKQNFWDHTNKLQSYEMEICESGASSCTSKQGNTNKYDVSYTYIECGPQALPFTEQVVSVSGTTYNSVKCPNDYSVLFGFGMATSSGRHQSALYSYFTPCRPGLKSCSLNMNEHDDKSYIYLVCVDATIWTGLNALSMIAKDDLHSAVNRYQQFNDGELVVTCPSEGTILTGFYGETHTSSPYVTVPFGKCAKSLKACSVHGSGQAIGIHNYRTLFTVALCKNN

SEQ ID NO: 56 - protein, 843 aa, unidentified
  REGION 1..843: note = misc_feature - SPECT2
  source 1..843: note = Plasmodium vivax
MKPRNINSLLAIWCILFSICEYGYVGSLRIGLRRNYSQGDSSDRLGGSDVDVRRSGELMDFGKNANILCNNISCNSKKEASFLSQKKQLEDDDDDELAALYNDDDDDASTTTGGGSETSDDDELELPDQDEGADEGEAEDQLATQEDSDNSGTDQGLKKKVSLSRHEKLIEDKKQQTENTFKKYRFGDEEEESEEKSPGKSKSLDPSSLDDDDGEGDDDDDGDEREKKQSNTRKAMKKDLDVFPGLYFAGIGYDSLFGNPLGEADSLTDPGYRGQIILMNWELSNKGVANDLATLQPLNGWIRKENACSRAESIKECSSVSDYTKNLTAEASVSGSYMGFGAFSASTGYKKFLQEASKRTSKTYLVKSNCVKYTVGLPPYVRWEQTTAFKNAVNGLPPHFTGLEADSECASDVYEQKKTSEECETVHAWIRFFKTYGTHVIMEAQLGGKITKIIRVENSSVNQMKKDGVSVKAQIKAQFGFASVGGSTNVSSDHSSKKNEDNYEMSEQLVVIGGNPIKDVTKEENLYEWSKTVSTNPMPINIKLLPISTIFDSDDLKNSYEKALIYYTRLYGFSPHDTMQKDEKDIVKILTASTTVTKTGPPPISAECPHNMVVLFGFVVKQNFWDHTNKLQSYEMEICESGASSCTSKQGNTNKYDVSYTYIECGPQALPFTEQVVSVSGTTYNSVKCPNDYSVLFGFGMATSSGRHQSALYSYFTPCRPGLKSCSLNMNEHDDKSYIYLVCVDATIWTGLNALSMIAKDDLHSAVNRYQQFNDGELVVTCPSEGTILTGFYGETHTSSPYVTVPFGKCAKSLKACSVHGSGQAIGIHNYRTLFTVALCKNN

SEQ ID NO: 57 - DNA, 2478 bp, unidentified
  misc_feature 1..2478: note = GPI_P113
  source 1..2478: mol_type = unassigned DNA; note = Plasmodium berghei
ggatccgccaccatggctaagatcttcctgtttagtttcttcttcgtatggttccagtattgctattctaagaacccctcagactacgctcatagcattgtctctaattttgagtccgagaatacgttgaagtgtttgaaaggcaacgtgtatatacttcaatgtcagatcaagtgcatgaactcaaacaacgagatcatatacaaggagtgtctcaacgatatcgaaaagatttgcaaagataagaaaacatgcagttattacttcgactacatcttcaaaaccaagaatcacaagcttagaaacaacaacaataacaacatctacatcgacaattgcatcgattccgacaagaatgagatcaaatctaccttcacctgtgtcctcaatccattgctggaatttgacaataacaaccacgtcatttataacttcctgctgaacaacaagaacaatgacaagatcgtctgtaaaaatagcaacatatatattaacaacgctactattcactataccttctccgacattaagtttaaagacgtgactagctacataaaagagaaatgtaacgagaaaactaattgcgtgataaacccctattctatccaaaccgatatcctgaatgagaagaacgacgcatatctgctgaactcatacatcagcatatccttcgcatgtgtgaaaattaacctggaaagctatctgtacggaggtgatatcgatgaatttgatcagataaacgatgaagaaaatgaggacaataagtacctggatcacaatgatctcgatgaaaaaaatgaggaaattatttccctgaagaacgaaatcaacgacatcctgaatgacgagaagatcgataacatcgccgagaagctgaagattgcgaaatttacaataagcaagaaaatcaacgaggagatcaagaagaagaacgacatctttaacaacctggccaacgacatctaccagttcatcggtaacgagtactactttacttccgacatcaaagacatgattgaagataggtacaatgaactgaacaaaacatctcagtctgacttgtattacatttaccttctgaatgtgtttgacattgaaaagatttacgggatctacctgtctagttatcaggagcgactgcagcagatcctgcaaaccaacatgacaaatctggactatgttgagaagaagattggaagcttgcggaacatttacatgttcctgtataaaaagagtaagaagtataacgcgctggatatcttcgacgaatactacgattatgtgctcaattacaatgacttcgctaaagacaatgagataatcagcgccgacattttcattaagtcaaaacctgatatccctcaactcaatttcgagatcaacaatgagaacaagaacgtgaagtataaagacgttaccgatcttgacgagctggataacctcaacaggataaatagaattatcaatatccggaatgtgctggtcaagcagctcaagatcctttacaaccagagaaataacatatttatcaaacaggccatgctggtgaaatcatactgctacaaaaatccactggatattacggatttcagcagcatcttcaagaataactacaataagttgaaatacgacgcctataaggagggcaatggacacattaacgtggccgacaaaatcaatccaaactttgtcgtgaaatacctgaacaatctttataaacagcatgttaacaagaattatatcctgaacagctgggaccctaaatacaatcgcatgaataagaagattaagatcattctcattttggggtatggccaggtaatccagatcgagaaacagattaaccgtcatataggcaagtacaacgccctgcttgaaaaggcaaccctctataacgtgggaaatctctttacacagactacgaatattttgaacgacatctcaggctcactgaacgatgggcttgacccgaacatccatgatcaggaggatgtcactgtcgttgaatcctctgaaagtaacaagctcgcagaacctgaggaacccattgagaaggtagaggttgatagggtagagaaatccgatgatgccaataatgccactcaacaagtgacaggaacagacgaggccaattacgatacagctagcgggaatagtaccaacatcaacattgacaacatagactatggtgtggacgaatctatccgaattattaagtactccaaggctgaggaggatgaatataatgagagcggcaataacgaaaatgaaaacaacgagaataacgagaacgagaacaatgaaaatgagaacaacgagaatgagaataatgagaatgaaaataacgagaacgaaaatatagaactgaagaatatagaacacgaaaacaaatccaacgcatctagcgcttccctcagtaacattttctttacctttatcattgcagctctgtttatccgcccctttctgtgactcgag

SEQ ID NO: 58 - protein, 819 aa, unidentified
  REGION 1..819: note = misc_feature - GPI_P113
  source 1..819: note = Plasmodium berghei
MAKIFLFSFFFVWFQYCYSKNPSDYAHSIVSNFESENTLKCLKGNVYILQCQIKCMNSNNEIIYKECLNDIEKICKDKKTCSYYFDYIFKTKNHKLRNNNNNNIYIDNCIDSDKNEIKSTFTCVLNPLLEFDNNNHVIYNFLLNNKNNDKIVCKNSNIYINNATIHYTFSDIKFKDVTSYIKEKCNEKTNCVINPYSIQTDILNEKNDAYLLNSYISISFACVKINLESYLYGGDIDEFDQINDEENEDNKYLDHNDLDEKNEEIISLKNEINDILNDEKIDNIAEKLKIAKFTISKKINEEIKKKNDIFNNLANDIYQFIGNEYYFTSDIKDMIEDRYNELNKTSQSDLYYIYLLNVFDIEKIYGIYLSSYQERLQQILQTNMTNLDYVEKKIGSLRNIYMFLYKKSKKYNALDIFDEYYDYVLNYNDFAKDNEIISADIFIKSKPDIPQLNFEINNENKNVKYKDVTDLDELDNLNRINRIINIRNVLVKQLKILYNQRNNIFIKQAMLVKSYCYKNPLDITDFSSIFKNNYNKLKYDAYKEGNGHINVADKINPNFVVKYLNNLYKQHVNKNYILNSWDPKYNRMNKKIKIILILGYGQVIQIEKQINRHIGKYNALLEKATLYNVGNLFTQTTNILNDISGSLNDGLDPNIHDQEDVTVVESSESNKLAEPEEPIEKVEVDRVEKSDDANNATQQVTGTDEANYDTASGNSTNINIDNIDYGVDESIRIIKYSKAEEDEYNESGNNENENNENNENENNENENNENENNENENNENENIELKNIEHENKSNASSASLSNIFFTFIIAALFIRPFL

SEQ ID NO: 59 - protein, 818 aa, unidentified
  REGION 1..818: note = misc_feature - P113
  source 1..818: note = Plasmodium berghei
MKIFLFSFFFVWFQYCYSKNPSDYAHSIVSNFESENTLKCLKGNVYILQCQIKCMNSNNEIIYKECLNDIEKICKDKKTCSYYFDYIFKTKNHKLRNNNNNNIYIDNCIDSDKNEIKSTFTCVLNPLLEFDNNNHVIYNFLLNNKNNDKIVCKNSNIYINNATIHYTFSDIKFKDVTSYIKEKCNEKTNCVINPYSIQTDILNEKNDAYLLNSYISISFACVKINLESYLYGGDIDEFDQINDEENEDNKYLDHNDLDEKNEEIISLKNEINDILNDEKIDNIAEKLKIAKFTISKKINEEIKKKNDIFNNLANDIYQFIGNEYYFTSDIKDMIEDRYNELNKTSQSDLYYIYLLNVFDIEKIYGIYLSSYQERLQQILQTNMTNLDYVEKKIGSLRNIYMFLYKKSKKYNALDIFDEYYDYVLNYNDFAKDNEIISADIFIKSKPDIPQLNFEINNENKNVKYKDVTDLDELDNLNRINRIINIRNVLVKQLKILYNQRNNIFIKQAMLVKSYCYKNPLDITDFSSIFKNNYNKLKYDAYKEGNGHINVADKINPNFVVKYLNNLYKQHVNKNYILNSWDPKYNRMNKKIKIILILGYGQVIQIEKQINRHIGKYNALLEKATLYNVGNLFTQTTNILNDISGSLNDGLDPNIHDQEDVTVVESSESNKLAEPEEPIEKVEVDRVEKSDDANNATQQVTGTDEANYDTASGNSTNINIDNIDYGVDESIRIIKYSKAEEDEYNESGNNENENNENNENENNENENNENENNENENNENENIELKNIEHENKSNASSASLSNIFFTFIIAALFIRPFL

SEQ ID NO: 60 - DNA, 2919 bp, synthetic construct
  misc_feature 1..2919: note = PfP113 human CO
  source 1..2919: mol_type = other DNA
gccaccatggctaagatcccgttctttattctgcacatcctcctcctgcagtttctgctttgtctgatacgctgttatgtgcacaatgatgtgattaagttcggtgaggagaatagcctgaagtgctctcagggaaacttgtatgtgttgcactgtgaggtgcagtgccttaatggcaataatgagattatccacaagaggtgtaatgacgacattgagaaaaagtgcaacggcaataataaatgcatatacttctttgagtacgaactgcggaagaaaacacaaagctttcgaaataagaattctatcgagatttccgagtgtgtcgaaagcgagcagaacgaagtgaaaacctcaactacctgtctcctgagcaattcctttattcttgacgaggcctttatacagtatttcttcttcataaagaacaagaacgaagagcctgtgatttgtaaggatgggaatatcaacattaagagtgcactcctgcactctccgttctgtgaaatcaaactcaaggacatttccgaatatatacgcaaaaagtgtgacaacaacaaggaatgccttatagatcctctcgatgttcagaagaatttgctgaacgaagaagatccctgctacatcaataacagttacgtatctgtgaatgtggtctgcaacaaagaggaggagataggggatgagagcactgacagctcatcaatggagatccaggactcaacatcaaatgagcaagacgagaatgttaaaggaatgtcaagcagccaagagatgaactcaaacaacgatgaaaacaaaaaccaagacaacgaaagcgacgatgacgtcaataataataacaacaataacaatgatgaccaggacgagcaaggaaacgatggcgatgtcaccagctctatgaacaagaatgaggacaacaaggatttggagcatggttcctccaatgatgtcaataacaacactgacaccttggttaacaacaaagagaataaggagttcgtccttaaagagaagtctagccttacttctaaaattaacaaagagctggctcatagaactgccctgtttaacaaacttgcagacaacatatcacttctgcttaacaagaaatacgattccttcgaaattaaggatgtgctggaagataggtacaacgagatgaagagggacgcaaaccccgatgtctactacatatacctgatggatactctggatattgaaaagatcgaagatatcaacctggaagaggttaagatgtctctgctggcatcactgaaagaaacgatgaacaaaattgatacgatcgaaaagaaaatcgaagaatttaagaacaagtacatctccttgtataacaaggtgaaaaccacaatgcccgaactctttgacctgaatgaggatcttgtactgctctacaacgattttccctttgacaacggcatgatcagctccgacatcttctttaagtacaatccttccgagaacatcatggatcatcaggaaatggtgaagaaagggagtatcaccgaagatgaactcaggattgttaacgatcttgagccactggataactatagacgtcgtaaacgaattacagagctgagaaagattctggtggagaagctgcggattctgtacctggagaagaacaatctcttcaatacacaggcgagttgcatcaagagctattgctataagaatcctctgaacctcaaaaccttggaagtgctcttgaagaagaactactatagactgaaggagaataaagattacgatgttgtatccagcattatccagcatctcgacaatgtagacgccaacaagaagaagaaatggctgacccatgaacggatactgaagaagctccaagttctgattgccgaaggctataagcggatcaacgagaaagaaaaggacatcgaccgaagaatggctgtctacaatgccctctatgagaaagcacagtcttacaacctgcagaagcttttcaacgactccaacgattttctgaagaaatatgccataatgggaaacagtttcgacgacggcgatgaggttttcggttcccaaagctcaaactttaacatcttcgatagcaataacaccgaccagaacaatgagcaagagcagccaaagcaagatgaccagcttttgaacaataataacgatgacgtgctctcagagtcaaacaatgagaataaagagaaaacaagtgatgacgctactcataaggagactcaggagaaaagcgaccaggaaccttcccagaacattcaggaggacaactccgatgagaaacatgccgaaaacgaggagaacgtagaacagatcgaaactgatagtaatgtcagcgaagaagccaatgacgagaataaggataacatgcagacaaccactgacgaaggaaccgaagaacttcagcagaatgacgaagatgcggagagtctgaccaaggagaattccaaatctgaggagcaggagaatgaagattctactgacgccgaggcgattgataaagaggaagttgaaacggaagagaagggaaaggacgaacagaagaaagatgagcagaaggagcaggatgaggaagaggatggagagaaagaaaataagcacaagagctccgaaactaccaatgagactgtgaccgacatcgaggaaaataagaacgaggtcaaaggtgaggagcatcttcaggggtctgagcagagcattgaggcttccgaatcatcccagaaagatgagactaaagaaacagaggacaaggaggaatacgtgaacgctaatgatgacgaatcttctgaggaggacacgacgcctaacgagacaaataaaaccgacaacgggtcaagtttcttcttcgcgatgagcaatgcacttctcgtgatcttgctgttgcttttcatcgaattcctgtaa

SEQ ID NO: 61 - protein, 970 aa, unidentified
  REGION 1..970: note = misc_feature - PfP113
  source 1..970: note = Plasmodium falciparum
MAKIPFFILHILLLQFLLCLIRCYVHNDVIKFGEENSLKCSQGNLYVLHCEVQCLNGNNEIIHKRCNDDIEKKCNGNNKCIYFFEYELRKKTQSFRNKNSIEISECVESEQNEVKTSTTCLLSNSFILDEAFIQYFFFIKNKNEEPVICKDGNINIKSALLHSPFCEIKLKDISEYIRKKCDNNKECLIDPLDVQKNLLNEEDPCYINNSYVSVNVVCNKEEEIGDESTDSSSMEIQDSTSNEQDENVKGMSSSQEMNSNNDENKNQDNESDDDVNNNNNNNNDDQDEQGNDGDVTSSMNKNEDNKDLEHGSSNDVNNNTDTLVNNKENKEFVLKEKSSLTSKINKELAHRTALFNKLADNISLLLNKKYDSFEIKDVLEDRYNEMKRDANPDVYYIYLMDTLDIEKIEDINLEEVKMSLLASLKETMNKIDTIEKKIEEFKNKYISLYNKVKTTMPELFDLNEDLVLLYNDFPFDNGMISSDIFFKYNPSENIMDHQEMVKKGSITEDELRIVNDLEPLDNYRRRKRITELRKILVEKLRILYLEKNNLFNTQASCIKSYCYKNPLNLKTLEVLLKKNYYRLKENKDYDVVSSIIQHLDNVDANKKKKWLTHERILKKLQVLIAEGYKRINEKEKDIDRRMAVYNALYEKAQSYNLQKLFNDSNDFLKKYAIMGNSFDDGDEVFGSQSSNFNIFDSNNTDQNNEQEQPKQDDQLLNNNNDDVLSESNNENKEKTSDDATHKETQEKSDQEPSQNIQEDNSDEKHAENEENVEQIETDSNVSEEANDENKDNMQTTTDEGTEELQQNDEDAESLTKENSKSEEQENEDSTDAEAIDKEEVETEEKGKDEQKKDEQKEQDEEEDGEKENKHKSSETTNETVTDIEENKNEVKGEEHLQGSEQSIEASESSQKDETKETEDKEEYVNANDDESSEEDTTPNETNKTDNGSSFFFAMSNALLVILLLLFIEFL

SEQ ID NO: 62 - protein, 969 aa, unidentified
  REGION 1..969: note = misc_feature - PfP113
  source 1..969: note = Plasmodium falciparum
MKIPFFILHILLLQFLLCLIRCYVHNDVIKFGEENSLKCSQGNLYVLHCEVQCLNGNNEIIHKRCNDDIEKKCNGNNKCIYFFEYELRKKTQSFRNKNSIEISECVESEQNEVKTSTTCLLSNSFILDEAFIQYFFFIKNKNEEPVICKDGNINIKSALLHSPFCEIKLKDISEYIRKKCDNNKECLIDPLDVQKNLLNEEDPCYINNSYVSVNVVCNKEEEIGDESTDSSSMEIQDSTSNEQDENVKGMSSSQEMNSNNDENKNQDNESDDDVNNNNNNNNDDQDEQGNDGDVTSSMNKNEDNKDLEHGSSNDVNNNTDTLVNNKENKEFVLKEKSSLTSKINKELAHRTALFNKLADNISLLLNKKYDSFEIKDVLEDRYNEMKRDANPDVYYIYLMDTLDIEKIEDINLEEVKMSLLASLKETMNKIDTIEKKIEEFKNKYISLYNKVKTTMPELFDLNEDLVLLYNDFPFDNGMISSDIFFKYNPSENIMDHQEMVKKGSITEDELRIVNDLEPLDNYRRRKRITELRKILVEKLRILYLEKNNLFNTQASCIKSYCYKNPLNLKTLEVLLKKNYYRLKENKDYDVVSSIIQHLDNVDANKKKKWLTHERILKKLQVLIAEGYKRINEKEKDIDRRMAVYNALYEKAQSYNLQKLFNDSNDFLKKYAIMGNSFDDGDEVFGSQSSNFNIFDSNNTDQNNEQEQPKQDDQLLNNNNDDVLSESNNENKEKTSDDATHKETQEKSDQEPSQNIQEDNSDEKHAENEENVEQIETDSNVSEEANDENKDNMQTTTDEGTEELQQNDEDAESLTKENSKSEEQENEDSTDAEAIDKEEVETEEKGKDEQKKDEQKEQDEEEDGEKENKHKSSETTNETVTDIEENKNEVKGEEHLQGSEQSIEASESSQKDETKETEDKEEYVNANDDESSEEDTTPNETNKTDNGSSFFFAMSNALLVILLLLFIEFL

SEQ ID NO: 63 - DNA, 3141 bp, synthetic construct
  misc_feature 1..3141: note = PvP113 human CO
  source 1..3141: mol_type = other DNA
gccaccatggctaagctcccgcccctctgcaggttgccacttgcgctggtgcttctttgtctgacatccagagcgcgctgttatgtacacaacgacgtgatgaaatttggtgaagaaaactctctcaagtgttcacaagggtctctctacatccttcactgcgaggtcaagtgtgtgaatgcaaagaacagaatcatccaccgaagttgcatcgaccaggttgaggcgaagtgcatgggcaacgccaaatgtaagtactattttgactatgtcgtgaaatcccgcggacagagtttgcggaacaaaaatgaaatcgaaatagaagaatgcgtggaatccgaacggaatgagattaagacgtccacaacttgccttctgtcaaattcatttttgctggatgagacctacattcaatatttctttttcatcaaaaacaaaaatgaagaacccataacgtgtcgagacggtagactgagcgtaaagagtgcgatacttcatagtcccttttgcaaaataaatcttaaggacatcactgagattctcaagagacaatgcgatcacagtaaggagtgcgtcattaatccgtatgtcctccaaaaagatgcattgaatgagagagaccagtgttacattaacaactcatatgtctcactcaatgtcgtttgcaccaaagaaggggaggagcaaccagaagagagtggacataaacagaagagggatgacgacgtggacgaaaccgaggaaggctcatatgacgtctctgcggatcagaacaaatcagcaatagtcggcgagggaaacgatgatccggaatccctgggtgaagaagatgagctttcagagactaacgaggcagttgatctcattatgaactccaaggaaagctttgaaaacaaaattcggaaggcgaagtcaatacttctctcacagatgaacgagcaggaggtgaaaaagaatgcaatattcaagaaactcggggaagagctttccaaaatggtcctccagaaatatgaaccaagtgatctgaaagacttgatcgaggacaggtataatgaaatgagacggtcccctgaccaagacctttattatctctaccttatagatacactcgaaataaataaaatggaagatcttgacgtgactgcactgcaagaccagctcgctatattgttggaagagcagatgggcaagatgaatcgcattgagaaaacgattaaccgattgaggaaaaaatacctttctatttacaacaaagcgaagaacaagaaagttaaagacatttacgatgaaggagttgatcctgtactcacgtacgacgacttcgcgcacggcaacggcattattacggcagatattttctttaagtataagcctgccattaagccgttgacttttagcaaatctaatgcctctgaggagcgggggtcatccaaaaaaaaagagtacaaggaccttcttgagatggacgcgctggatgagtacaaccggaagaaacggataactgacatgcgaaacggtctgatggagactttgaagaaaatgtattacgcaaaaaatgggatattcaataatctggcgagttgcattaagtcatattgctataaaaggcctctcaaccttaatgcactttcctccgttcttaaaagaaattttgaaaatcttcgagagaaaaaatcaaccgatccagtggcccccatagtcagatatttgcaaaaggtcagtggcgaggttggcggggaagtgggaggcgcggctggcggtgctgcggggggtgctgcaagtggtgccgcatcaggagctgtcagtgaagcagtaggaggcgccataggtggggctgtgggcgaggcatctatcgcagtaaatccgccccgatgggaaaaatcacgacgaattcttcaaaagctcaaggctttgctgcacctgggttatcaacaggcactggacaaagagttggagatcgacgagaggactgacaaatacaacgctctgaatgagaaagcaaaagaatataaccttcagcggcttttttccgagtcagacaagttgcttaagaaggtcgcaatgcttacgtcagccagtgaaagtgcagatgaagtattcggtaaccaagcgagctttttcgatgtttacagaggagaggcagcgtcacaaaaaggagtagccgccagtgaaaagggcgtagctgcaagagagaaaggagtcgccgcgagtgagaagggggtcgcggcatcagaaaagggtgtggcggccagcgagaagggtgtcgcggcatcagaaaaaggtgttgccgcaagccaaaaaggcggagaaacgtctgaggaaggggaggctgccagtcaaaaggatgttgctacgtccgaggagggtggagctagcagcgagaaacgcgatgacgacgagatgaatccgcccgacgaagggtatgagtccgctaaggaagacggggagaatgcacaagatgacgacagcggcggaaatgctgaacccgtagagggaaaagcaggcgaaagtgaagacgctgatggggtaaatgccggctccaataaagaaggtgaggatggggagagcgttgaagaagaagctgcggaaggggaagcggcacccaaggaggaggctgcagacggagaagatgctccggaggaggaagccgagggtgaggatgccccagaagaagaggccgatggcgaagacgccccgaaggaggaggctactgacggagaggacgcccctaaagaggaagaggccgaaggtgaggatgcaagcaaggacgaggaggccgacgaaggctcaactgacgaggaagaagccgccgacggcgggagtactgatgctacagcagccgatgaagctgcaggcggagtcgcggaccaaaacgacgtgcccgttaagggagaagactcagatggcgcggaatctgatggagcagaagatgccgccactgaaattaggggtgaggctgaagccggtgaagaggccgcggagcaacctactggagaggccgtagtcaaaggggattccgagggcggggcttctgggctggaaaccgagaagaagggagacgacgggggctccttcttccaaggtctttcccgagtgttgcttactgttcttgcaatactttccttggaatttctcctttga

SEQ ID NO: 64 - protein, 1044 aa, unidentified
  REGION 1..1044: note = misc_feature - PvP113
  source 1..1044: note = Plasmodium vivax
MAKLPPLCRLPLALVLLCLTSRARCYVHNDVMKFGEENSLKCSQGSLYILHCEVKCVNAKNRIIHRSCIDQVEAKCMGNAKCKYYFDYVVKSRGQSLRNKNEIEIEECVESERNEIKTSTTCLLSNSFLLDETYIQYFFFIKNKNEEPITCRDGRLSVKSAILHSPFCKINLKDITEILKRQCDHSKECVINPYVLQKDALNERDQCYINNSYVSLNVVCTKEGEEQPEESGHKQKRDDDVDETEEGSYDVSADQNKSAIVGEGNDDPESLGEEDELSETNEAVDLIMNSKESFENKIRKAKSILLSQMNEQEVKKNAIFKKLGEELSKMVLQKYEPSDLKDLIEDRYNEMRRSPDQDLYYLYLIDTLEINKMEDLDVTALQDQLAILLEEQMGKMNRIEKTINRLRKKYLSIYNKAKNKKVKDIYDEGVDPVLTYDDFAHGNGIITADIFFKYKPAIKPLTFSKSNASEERGSSKKKEYKDLLEMDALDEYNRKKRITDMRNGLMETLKKMYYAKNGIFNNLASCIKSYCYKRPLNLNALSSVLKRNFENLREKKSTDPVAPIVRYLQKVSGEVGGEVGGAAGGAAGGAASGAASGAVSEAVGGAIGGAVGEASIAVNPPRWEKSRRILQKLKALLHLGYQQALDKELEIDERTDKYNALNEKAKEYNLQRLFSESDKLLKKVAMLTSASESADEVFGNQASFFDVYRGEAASQKGVAASEKGVAAREKGVAASEKGVAASEKGVAASEKGVAASEKGVAASQKGGETSEEGEAASQKDVATSEEGGASSEKRDDDEMNPPDEGYESAKEDGENAQDDDSGGNAEPVEGKAGESEDADGVNAGSNKEGEDGESVEEEAAEGEAAPKEEAADGEDAPEEEAEGEDAPEEEADGEDAPKEEATDGEDAPKEEEAEGEDASKDEEADEGSTDEEEAADGGSTDATAADEAAGGVADQNDVPVKGEDSDGAESDGAEDAATEIRGEAEAGEEAAEQPTGEAVVKGDSEGGASGLETEKKGDDGGSFFQGLSRVLLTVLAILSLEFLL

SEQ ID NO: 65 - protein, 969 aa, unidentified
  REGION 1..969: note = misc_feature - P113
  source 1..969: note = Plasmodium vivax
MKLPPLCRLPLALVLLCLTSRARCYVHNDVMKFGEENSLKCSQGSLYILHCEVKCVNAKNRIIHRSCIDQVEAKCMGNAKCKYYFDYVVKSRGQSLRNKNEIEIEECVESERNEIKTSTTCLLSNSFLLDETYIQYFFFIKNKNEEPITCRDGRLSVKSAILHSPFCKINLKDITEILKRQCDHSKECVINPYVLQKDALNERDQCYINNSYVSLNVVCTKEGEEQPEESGHKQKRDDDVDETEEGSYDVSADQNKSAIVGEGNDDPESLGEEDELSETNEAVDLIMNSKESFENKIRKAKSILLSQMNEQEVKKNAIFKKLGEELSKMVLQKYEPSDLKDLIEDRYNEMRRSPDQDLYYLYLIDTLEINKMENLDVTALQDQLAILLEEQMGKMNRIEKTINRLRKKYLSIYNKAKNKKVKDIYDEGVDPVLTYDDFAHGNGIITADIFFKYKPAIKPLTFSKSNASEERGSSKKKEYKDLLEMDALDEYNRKKRITDMRNGLMETLKKMYYAKNGIFNNLASCIKSYCYKRPLNLNALSSVLKRNFENLREKKSTDPVAPIVRYLQKKSRRILQKLKALLHLGYQQALDKELEIDERTDKYNALNEKAKEYNLQRLFSESDKLLKKVAMLTSASESADEVFGNQASFFDVYRGEAASEKGVAASQKGGETSEEGEAASQKDVATSEEGGASSEKRDDDEMNPPDEGYESAKEDGENAQDDDSGGNAEPVEGKAGESEDADGVNAGSNKEGEDGESVEEEAAEGEAAPKEEAADGEDAPEEEAEGEDAPKEEAADGEASSKEEETDGEVAPEEAADGEDAPKEEATDGEDAPKEEEAEGEDASKDEEADEGSTDEEEAADGGSTDATAADEAAGGVADQNDVPVKGEDSDGAESDGAEDAATEIRGEAEAGEEAAEQPTGEAVVKGDSEGGASGLETEKKGDDGGSFFQGLSRVLLTVLAILSLEFLL

SEQ ID NO: 66 - DNA, 654 bp, unidentified
  misc_feature 1..654: note = Ag40
  source 1..654: mol_type = unassigned DNA; note = Plasmodium berghei
ggatccgccaccatggctggggcgagtatgagccacttgcagtgtctgacatctgttgctggtctgtcctctatcgtcatgtcaatgttccccaaactcattgccaataatccttccctgtttagaccactgctcaacatttcctggggatatctgttcggaagcactgtatggctgtgcttcttcagtgagattgggttggtcaggagaatcaatgctcctaaacggaagaatctgccagagaatgcagaacaagccaaagaacagctgaaggagatcaagaacaacgaaggcgattttaaccgacgcaatatcgacttcaagtacttctttagcctttccacaatcttctctagcatactgctgcttagcacagtgaaactcgccaacaacaatctgcagttgaggatctgttccaccattgtgtcactgagttgcatactgaacaatatgtactttcagaacaagatacactcacttgcactgaagaaagagagtctctttaaggacatgatcgatcgtccgaaagataccactattctggtgaacctgaagaagaacaagaccgactttcacatccatcatggcctttctctgctcttgctctatagcagcttctttggcctcactccctacattttcacgtgactcgag

SEQ ID NO: 67 - protein, 211 aa, unidentified
  REGION 1..211: note = misc_feature - Ag40
  source 1..211: note = Plasmodium berghei
MAGASMSHLQCLTSVAGLSSIVMSMFPKLIANNPSLFRPLLNISWGYLFGSTVWLCFFSEIGLVRRINAPKRKNLPENAEQAKEQLKEIKNNEGDFNRRNIDFKYFFSLSTIFSSILLLSTVKLANNNLQLRICSTIVSLSCILNNMYFQNKIHSLALKKESLFKDMIDRPKDTTILVNLKKNKTDFHIHHGLSLLLLYSSFFGLTPYIFT

SEQ ID NO: 68 - protein, 210 aa, unidentified
  REGION 1..210: note = misc_feature - Ag40
  source 1..210: note = Plasmodium berghei
MGASMSHLQCLTSVAGLSSIVMSMFPKLIANNPSLFRPLLNISWGYLFGSTVWLCFFSEIGLVRRINAPKRKNLPENAEQAKEQLKEIKNNEGDFNRRNIDFKYFFSLSTIFSSILLLSTVKLANNNLQLRICSTIVSLSCILNNMYFQNKIHSLALKKESLFKDMIDRPKDTTILVNLKKNKTDFHIHHGLSLLLLYSSFFGLTPYIFT

SEQ ID NO: 69 - DNA, 642 bp, synthetic construct
  misc_feature 1..642: note = PfAg40 human CO
  source 1..642: mol_type = other DNA
gccaccatggctgggtgtacagtctctaatctcaaatgcgtgaccaatgtggcaggactggcaagtctggttatcagtctgtttccgaaactcatcataaagaacccacaagtgcttcgaccactgctgaatgtgtcctggggttatctgtttggtagcaccttttggctgtgcttcttctccgaagtaggactgcttcgcagcctgaagaacatgaaaggggtacctttgcctgaatcagccagtgaggcgaagaagcttctcgaagagatgaagaactctgagggcgatttcaatcggagatcactggacttccagtacttcttttccctcgctacgttgttctcaggcattctgttgctgagcacagtgaagttggccaaccataacctgcagcttaggcttagtagctctgtggtcgtcatcacatcactgctgaatagcctgtatctgcacaataaagtgcataatctgaaaagcaagaaagaaagcctctataacgactttattgccaatcccaagaacgagaaaactgtcgctgatctgaagaagaacaagaaagagtttcacatctttcacggattgtccgttctctctctctacgtttccttcttcggcctgactccctacattttcacctaa

SEQ ID NO: 70 - protein, 211 aa, unidentified
  REGION 1..211: note = misc_feature - PfAg40
  source 1..211: note = Plasmodium falciparum
MAGCTVSNLKCVTNVAGLASLVISLFPKLIIKNPQVLRPLLNVSWGYLFGSTFWLCFFSEVGLLRSLKNMKGVPLPESASEAKKLLEEMKNSEGDFNRRSLDFQYFFSLATLFSGILLLSTVKLANHNLQLRLSSSVVVITSLLNSLYLHNKVHNLKSKKESLYNDFIANPKNEKTVADLKKNKKEFHIFHGLSVLSLYVSFFGLTPYIFT

SEQ ID NO: 71 - protein, 210 aa, unidentified
  REGION 1..210: note = misc_feature - Ag40
  source 1..210: note = Plasmodium falciparum
MGCTVSNLKCVTNVAGLASLVISLFPKLIIKNPQVLRPLLNVSWGYLFGSTFWLCFFSEVGLLRSLKNMKGVPLPESASEAKKLLEEMKNSEGDFNRRSLDFQYFFSLATLFSGILLLSTVKLANHNLQLRLSSSVVVITSLLNSLYLHNKVHNLKSKKESLYNDFIANPKNEKTVADLKKNKKEFHIFHGLSVLSLYVSFFGLTPYIFT

SEQ ID NO: 72 - DNA, 639 bp, synthetic construct
  misc_feature 1..639: note = PvAg40 human CO + kozac
  source 1..639: mol_type = other DNA
gccaccatgggggcgacggtatcatatcttagatgcgtgaccagtatagcagggctgagcagcctcgtgctgtctttgtttcccaagctgattatgaaaaaccctcaggtactccgaccactccttaacattagctggggttatttgtttggctcaaccttttggttgtgtttgttctccgaagtaggacttttccggtccctgaaaaatatgaagcgcataccaatccctgaaaacgcagaagaggctaagaagcaattggaggagatgaaaagcatggaaggggattttaccaggcgcagggaagatttccaatatttttttggtttttccaccttgttttctggtattcttcttctcagtacggtaagacttgcgaatcacaacatgcaactgaggatttccagtaccatcgttgcccttagctgcctgctcaataacttgtaccttcagaataaggtacattctcttaaaatccaaaaagaaaacctgtacaacgaactcatccgcaatcctaagtcagagacgactatagcggagattaagaaaaacaaaaaagatttccatatataccacggcttgtccctgttgtccctttacataagcttcctcggccttactccatatatatttacctag

SEQ ID NO: 73 - protein, 210 aa, unidentified
  REGION 1..210: note = misc_feature - PvAg40
  source 1..210: note = Plasmodium vivax
MGATVSYLRCVTSIAGLSSLVLSLFPKLIMKNPQVLRPLLNISWGYLFGSTFWLCLFSEVGLFRSLKNMKRIPIPENAEEAKKQLEEMKSMEGDFTRRREDFQYFFGFSTLFSGILLLSTVRLANHNMQLRISSTIVALSCLLNNLYLQNKVHSLKIQKENLYNELIRNPKSETTIAEIKKNKKDFHIYHGLSLLSLYISFLGLTPYIFT

SEQ ID NO: 74 - protein, 210 aa, unidentified; identical to SEQ ID NO: 73
  REGION 1..210: note = misc_feature - Ag40
  source 1..210: note = Plasmodium vivax

SEQ ID NO: 75 - DNA, 1077 bp, unidentified
  misc_feature 1..1077: note = Ag45
  source 1..1077: mol_type = unassigned DNA; note = Plasmodium berghei
ggatccgccaccatggcttcttactcaaactcttccattaagcagaaatccgatagtgtgagtgtctacaatactcggactggaaatgtcagtaaaactcgcttgatccgtctgcaaaatgggcattaccgtagagtggtcgacattagcaataaggacgagaaggagattctcttcaggacatgtgcttgcgcttgtccaacacctcgaaatgaggagacacgcaaaacctatatgccacctctgaacaatgtgtctaccgtagcgtatagaaagcggatctattcttcctttgggaataaggacggtaacgatacaggcaacaacgagagcataacagaacatgaggacccgattaggaccttttccgaaacgacaagtaggcaggaaagtaccatcgacgacaaaacggagactagcatcaatagcaaggaaacagatgatggcaaccagtttggaaggttgtttgaagaactggaggagaaagaggatgaactgattgaggaaaaggaggaggagctgatagaagagaaggaggaggaacttatagaggagaaagaagaagagctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaatgactcgag

SEQ ID NO: 76 - protein, 352 aa, unidentified
  REGION 1..352: note = misc_feature - Ag45
  source 1..352: note = Plasmodium berghei
MASYSNSSIKQKSDSVSVYNTRTGNVSKTRLIRLQNGHYRRVVDISNKDEKEILFRTCACACPTPRNEETRKTYMPPLNNVSTVAYRKRIYSSFGNKDGNDTGNNESITEHEDPIRTFSETTSRQESTIDDKTETSINSKETDDGNQFGRLFEELEEKEDELIEEKEEELIEEKEEELIEEKEEELIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKK

SEQ ID NO: 77 - protein, 351 aa, unidentified
  REGION 1..351: note = misc_feature - Ag45
  source 1..351: note = Plasmodium berghei
MSYSNSSIKQKSDSVSVYNTRTGNVSKTRLIRLQNGHYRRVVDISNKDEKEILFRTCACACPTPRNEETRKTYMPPLNNVSTVAYRKRIYSSFGNKDGNDTGNNESITEHEDPIRTFSETTSRQESTIDDKTETSINSKETDDGNQFGRLFEELEEKEDELIEEKEEELIEEKEEELIEEKEEELIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKK

SEQ ID NO: 78 - DNA, 1197 bp, synthetic construct
  misc_feature 1..1197: note = PfAg45 human CO
  source 1..1197: mol_type = other DNA
gccaccatggcttcagattactttacaattctgtccaatattttcacaagcactagcctgaagaagaaatacagttcccggttgagcacaaaatccaagaagaaccaaaagcgagtcaaactgataagactgcggaatggacattttcgccgaattgtggatatttccaacattgacgagaagagcatcttccccagaagctgtacttttgcgtcaattagcagtgctagcaaagaaaacgagaggaagaattcaagcgaggacacaaaagaacctcaggagaatctgtatggcaaatcaaacacttcaagctctatcacgataaagatcaatttcgacgaaagcgatgagaacaagagtgatcaggataaccactctatcgataccattagcgacatctcttttacccagacttcacgcaaatctcttgaaattgaaagtaatacctatgagagttatcgcgaagtggagaaggaggacattgaggaggaggaggaagaggagaaagaagaagaatatgaggaggaagaagaagaagaggaatacgaagaggaagaggaagaagaggaggaagagtatgaggaggagggtctgaaaaccgaagaggagaaggaagaagataataaggaggtagagccagaagaggagcttaaagaagaagatgacaaggaggttgagcctgaggaggagaaggagaatgagcagaagaaagaagaacaagaggagaataacctcgaagctcccagcaaaacactgatgaaaggggttaagaccaacatatacttcctgtctaccaaagagcggatagaagcactcatgtgctacaactacatatccaacgccattattttcgaaaagggcaagtttctccgttatatcttcatgaacaatgtcaacaatatcatcgtgaacgagcacatgatcaatatgttgtgcaagaaggaaaagatcaaatacatcctgtcatctaactccatcatcattgaaagcaacgacttcatcaaaccgctcatcattgagtttgacagtaacatctctaagaagatctttgtcaaacacttgaaaatggtggactccttcaaactggatgacaagctgtacagggagtacctgaatgacctttctgaacatgagagggatagactgaaacatgtggagtccttctattccaatgccataaaggtgcacaatacgtaa

SEQ ID NO: 79 - protein, 396 aa, unidentified
  REGION 1..396: note = misc_feature - PfAg45
  source 1..396: note = Plasmodium falciparum
MASDYFTILSNIFTSTSLKKKYSSRLSTKSKKNQKRVKLIRLRNGHFRRIVDISNIDEKSIFPRSCTFASISSASKENERKNSSEDTKEPQENLYGKSNTSSSITIKINFDESDENKSDQDNHSIDTISDISFTQTSRKSLEIESNTYESYREVEKEDIEEEEEEEKEEEYEEEEEEEEYEEEEEEEEEEYEEEGLKTEEEKEEDNKEVEPEEELKEEDDKEVEPEEEKENEQKKEEQEENNLEAPSKTLMKGVKTNIYFLSTKERIEALMCYNYISNAIIFEKGKFLRYIFMNNVNNIIVNEHMINMLCKKEKIKYILSSNSIIIESNDFIKPLIIEFDSNISKKIFVKHLKMVDSFKLDDKLYREYLNDLSEHERDRLKHVESFYSNAIKVHNT

SEQ ID NO: 80 - protein, 395 aa, unidentified
  REGION 1..395: note = misc_feature - Ag45
  source 1..395: note = Plasmodium falciparum
MSDYFTILSNIFTSTSLKKKYSSRLSTKSKKNQKRVKLIRLRNGHFRRIVDISNIDEKSIFPRSCTFASISSASKENERKNSSEDTKEPQENLYGKSNTSSSITIKINFDESDENKSDQDNHSIDTISDISFTQTSRKSLEIESNTYESYREVEKEDIEEEEEEEKEEEYEEEEEEEEYEEEEEEEEEEYEEEGLKTEEEKEEDNKEVEPEEELKEEDDKEVEPEEEKENEQKKEEQEENNLEAPSKTLMKGVKTNIYFLSTKERIEALMCYNYISNAIIFEKGKFLRYIFMNNVNNIIVNEHMINMLCKKEKIKYILSSNSIIIESNDFIKPLIIEFDSNISKKIFVKHLKMVDSFKLDDKLYREYLNDLSEHERDRLKHVESFYSNAIKVHNT

SEQ ID NO: 81 - DNA, 1122 bp, synthetic construct
  misc_feature 1..1122: note = PvAg45 human CO + kozac
  source 1..1122: mol_type = other DNA
gccaccatggctaatttgagttctcccctcctggccttgcccgaggagggtaagaagcgaagaacaaaactcatccgactgaggaacggtcactataggcggatagtggacatttcaaataccgacgaacggaagctcattccttctatgtgccgctgcgcgtgtgtcactcccagaaaagacgaagtggagaatgagggtaagtgggaagacgctaagaaagcaaaatcaagccaggaatatgatgaaacttctgattatgttgaatctgaaaagaaagagagctatatgctcgcagtcaatgaggaggatcagacggaggatatgtactcaaagacgatcagctttacttctataacccctacatctataagatccgaagagccagagccaaggcggaaactctcccttctggatgttaaagaagaagaggaagaggaggaggaggaagaagaagaggaagaagaggaagaggaggaagaggaagaggaagaagaggaagagaaagaaaaagaaaaagagaaggaggaagaggaagaagaggaggaggaagaggaagaggaagaggaggaggaagaagaagaggaagatgaaatagaatctaccgcagaggaaaaggaagaagagaagaagcaagtcccaccggaaggtaagaaattgatcgaaccctcaaagacccttatgagagggactaagaccaacatttattttttgagtaataaggagatggttcaaactctgatgtgttataattataattgcaacgcagtggtattcgaaaaagacacttttttgaggtatctgtacatgaagagcatcagcaacatcatcctcaacgaacgaatgatagatgaactgtgcaaacaggaagatcttaaatacgtgcttacaagcaatgctatcgtgttggaatctactgactttcttaagcctcttataattgagtttgaatccagtattagtaaaagagttttcgtgcgccacctgaagcataacgctcgcaaggaaatcgacatgaaaaaatatcatgattacatgggcgaacttaacgctaacgagaagattaggctgatgaaaattgagcgatttcatagtttcaacaagatgatccaatgcaactag

SEQ ID NO: 82 - protein, 371 aa, unidentified
  REGION 1..371: note = misc_feature - PvAg45
  source 1..371: note = Plasmodium vivax
MANLSSPLLALPEEGKKRRTKLIRLRNGHYRRIVDISNTDERKLIPSMCRCACVTPRKDEVENEGKWEDAKKAKSSQEYDETSDYVESEKKESYMLAVNEEDQTEDMYSKTISFTSITPTSIRSEEPEPRRKLSLLDVKEEEEEEEEEEEEEEEEEEEEEEEEEEEKEKEKEKEEEEEEEEEEEEEEEEEEEEEDEIESTAEEKEEEKKQVPPEGKKLIEPSKTLMRGTKTNIYFLSNKEMVQTLMCYNYNCNAVVFEKDTFLRYLYMKSISNIILNERMIDELCKQEDLKYVLTSNAIVLESTDFLKPLIIEFESSISKRVFVRHLKHNARKEIDMKKYHDYMGELNANEKIRLMKIERFHSFNKMIQCN

SEQ ID NO: 83 - protein, 345 aa, unidentified
  REGION 1..345: note = misc_feature - Ag45
  source 1..345: note = Plasmodium vivax
MNLSSPLLALPEEGKKRRTKLIRLRNGHYRRIVDISNTDERKLIPSMCRCACVTPRKDEVENEGKWEDAKKAKSSQEYDETSDYVESEKKESYMLAVNEEDQTEDMYSKTISFTSITPTSIRSEEPEPRRKLSLLDVKEEEEEEEEEEEEEEEEEEEEEEEKEEEEEEDEIESTAEEKEEEKKQVPPEGKKLIEPSKTLMRGTKTNIYFLSNKEMVQTLMCYNYNCNAVVFEKDTFLRYLYMKSISNIILNERMIDELCKQEDLKYVLTSNAIVLESTDFLKPLIIEFESSISKRVFVRHLKHNARKEIDMKKYHDYMGELNANEKIRLMKIERFHSFNKMIQCN

SEQ ID NO: 84 - DNA, 12 bp, synthetic construct
  misc_feature 1..12: note = Kozac consensus sequence
  source 1..12: mol_type = other DNA
gccaccatggct

SEQ ID NO: 85 - DNA, 12 bp, synthetic construct
  misc_feature 1..12: note = Kozac consensus sequence
  source 1..12: mol_type = other DNA
gccaccatggcc

SEQ ID NO: 86-87 - skipped (2 entries)

SEQ ID NO: 88 - protein, 9 aa, synthetic construct
  REGION 1..9: note = CD8 T cell epitope
QAQRNLNEL

SEQ ID NO: 89 - protein, 9 aa, synthetic construct
  REGION 1..9: note = CD8 T cell epitope
SALLNVDNL

SEQ ID NO: 90 - protein, 9 aa, synthetic construct
  REGION 1..9: note = CD8 T cell epitope
KSPSNFTII

SEQ ID NO: 91 - protein, 9 aa, synthetic construct
  REGION 1..9: note = CD8 T cell epitope
SNQTNQETI

SEQ ID NO: 92 - protein, 9 aa, synthetic construct
  REGION 1..9: note = CD8 T cell epitope
ITPENKTLI

SEQ ID NO: 93 - protein, 9 aa, synthetic construct
  REGION 1..9: note = CD8 T cell epitope
KLIANNPSL

SEQ ID NO: 94 - protein, 9 aa, synthetic construct
  REGION 1..9: note = CD8 T cell epitope
SCILNNMYF

SEQ ID NO: 95 - DNA, 372 bp, synthetic construct
  misc_feature 1..372: note = DNA sequence of PD Plasmodium berghei ANKA 18-10NT
  source 1..372: mol_type = other DNA
ggatccgccaccatggacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattcctgactcgag

SEQ ID NO: 96 - protein, 117 aa, synthetic construct
  REGION 1..117: note = Amino acid sequence of PD Plasmodium berghei ANKA 18-10NT
MDNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNS

SEQ ID NO: 97 - DNA, 528 bp, synthetic construct
  misc_feature 1..528: note = DNA sequence of PD Plasmodium berghei ANKA 18-10CT
  source 1..528: mol_type = other DNA
ggatccgccaccatggctctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacagtgactcgag

SEQ ID NO: 98 - protein, 169 aa, synthetic construct
  REGION 1..169: note = Amino acid sequence of PD Plasmodium berghei ANKA 18-10CT
MALTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQ

SEQ ID NO: 99 - DNA, 1197 bp, synthetic construct
  misc_feature 1..1197: note = DNA sequence of PD Plasmodium falciparum 3D7 18-10 -SP
  source 1..1197: mol_type = other DNA
ggatccgccaccatggacaacaacagctacagcttcgagatcgtgaaccgcagcacctggctgaacatcgccgagcgcatcttcaagggcaacgcccccttcaacttcaccatcatcccctacaactacgtgaacaacagcaccgaggagaacaacaacaaggacagcgtgctgctgatcagcaagaacctgaagaacagcagcaaccccgtggacgagaacaaccacatcatcgacagcaccaagaagaacaccagcaacaacaacaacaacaacagcaacatcgtgggcatctacgagagccaggtgcacgaggagaagatcaaggaggacaacacccgccaggacaacatcaacaagaaggagaacgagatcatcaacaacaaccaccagatccccgtgagcaacatcttcagcgagaacatcgacaacaacaagaactacatcgagagcaactacaagagcacctacaacaacaaccccgagctgatccacagcaccgacttcatcggcagcaacaacaaccacaccttcaacttcctgagccgctacaacaacagcgtgctgaacaacatgcagggcaacaccaaggtgcccggcaacgtgcccgagctgaaggcccgcatcttcagcgaggaggagaacaccgaggtggagagcgccgagaacaaccacaccaacagcctgaaccccaacgagagctgcgaccagatcatcaagctgggcgacatcatcaacagcgtgaacgagaagatcatcagcatcaacagcaccgtgaacaacgtgctgtgcatcaacctggacagcgtgaacggcaacggcttcgtgtggaccctgctgggcgtgcacaagaagaagcccctgatcgaccccagcaacttccccaccaagcgcgtgacccagagctacgtgagccccgacatcagcgtgaccaaccccgtgcccatccccaagaacagcaacaccaacaaggacgacagcatcaacaacaagcaggacggcagccagaacaacaccaccaccaaccacttccccaagccccgcgagcagctggtgggcggcagcagcatgctgatcagcaagatcaagccccacaagcccggcaagtacttcatcgtgtacagctactaccgccccttcgaccccacccgcgacaccaacacccgcatcgtggagctgaacgtgcagtgactcgag

SEQ ID NO: 100 - protein, 392 aa, synthetic construct
  REGION 1..392: note = Amino acid sequence of PD Plasmodium falciparum 3D7 18-10 -SP
MDNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQ

SEQ ID NO: 101 - DNA, 528 bp, synthetic construct
  misc_feature 1..528: note = DNA sequence of PD Plasmodium berghei ANKA 11-10CT
  source 1..528: mol_type = other DNA
ggatccgccaccatggctctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaatgactcgag

SEQ ID NO: 102 - protein, 169 aa, synthetic construct
  REGION 1..169: note = Amino acid sequence of PD Plasmodium berghei ANKA 11-10CT
MALIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKK

SEQ ID NO: 103 - DNA, 561 bp, synthetic construct
  misc_feature 1..561: note = DNA sequence of PD Plasmodium falciparum 3D7 11-10CT
  source 1..561: mol_type = other DNA
ggatccgccaccatggaggaggacgacaaggaggtggagcccgaggaggagaaggagaacgagcagaagaaggaggagcaggaggagaacaacctggaggcccccagcaagaccctgatgaagggcgtgaagaccaacatctacttcctgagcaccaaggagcgcatcgaggccctgatgtgctacaactacatcagcaacgccatcatcttcgagaagggcaagttcctgcgctacatcttcatgaacaacgtgaacaacatcatcgtgaacgagcacatgatcaacatgctgtgcaagaaggagaagatcaagtacatcctgagcagcaacagcatcatcatcgagagcaacgacttcatcaagcccctgatcatcgagttcgacagcaacatcagcaagaagatcttcgtgaagcacctgaagatggtggacagcttcaagctggacgacaagctgtaccgcgagtacctgaacgacctgagcgagcacgagcgcgaccgcctgaagcacgtggagagcttctacagcaacgccatcaaggtgcacaactgactcgag

SEQ ID NO: 104 - protein, 180 aa, synthetic construct
  REGION 1..180: note = Amino acid sequence of PD Plasmodium falciparum 3D7 11-10CT
MEEDDKEVEPEEEKENEQKKEEQEENNLEAPSKTLMKGVKTNIYFLSTKERIEALMCYNYISNAIIFEKGKFLRYIFMNNVNNIIVNEHMINMLCKKEKIKYILSSNSIIIESNDFIKPLIIEFDSNISKKIFVKHLKMVDSFKLDDKLYREYLNDLSEHERDRLKHVESFYSNAIKVHN

SEQ ID NO: 105 - DNA, 747 bp, synthetic construct
  misc_feature 1..747: note = DNA sequence of PD Plasmodium berghei ANKA TRAP NT
  source 1..747: mol_type = other DNA
ggatccgccaccatggctaacggtcaggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggtttgactcgag

SEQ ID NO: 106 - protein, 242 aa, synthetic construct
  REGION 1..242: note = Amino acid sequence of PD Plasmodium berghei ANKA TRAP NT
MANGQEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKV

SEQ ID NO: 107 - DNA, 903 bp, synthetic construct
  misc_feature 1..903: note = DNA sequence of PD Plasmodium falciparum 3D7 TRAP NT
  source 1..903: mol_type = other DNA
ggatccgccaccatggacgtgcagaacaacatcgtggacgagatcaagtaccgcgaggaggtgtgcaacgacgaggtggacctgtacctgctgatggactgcagcggcagcatccgccgccacaactgggtgaaccacgccgtgcccctggccatgaagctgatccagcagctgaacctgaacgacaacgccatccacctgtacgccagcgtgttcagcaacaacgcccgcgagatcatccgcctgcacagcgacgccagcaagaacaaggagaaggccctgatcatcatcaagagcctgctgagcaccaacctgccctacggcaagaccaacctgaccgacgccctgctgcaggtgcgcaagcacctgaacgaccgcatcaaccgcgagaacgccaaccagctggtggtgatcctgaccgacggcatccccgacagcatccaggacagcctgaaggagagccgcaagctgagcgaccgcggcgtgaagatcgccgtgttcggcatcggccagggcatcaacgtggccttcaaccgcttcctggtgggctgccaccccagcgacggcaagtgcaacctgtacgccgacagcgcctgggagaacgtgaagaacgtgatcggccccttcatgaaggccgtgtgcgtggaggtggagaagaccgccagctgcggcgtgtgggacgagtggagcccctgcagcgtgacctgcggcaagggcacccgcagccgcaagcgcgagatcctgcacgagggctgcaccagcgagctgcaggagcagtgcgaggaggagcgctgcctgcccaagcgcgagcccctggacgtgcccgacgagcccgaggacgaccagccccgcccccgcggcgacaacttcgccgtggagaagcccaacgagaactgactcgag

SEQ ID NO: 108 - protein, 294 aa, synthetic construct
  REGION 1..294: note = Amino acid sequence of PD Plasmodium falciparum 3D7 TRAP NT
MDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCLPKREPLDVPDEPEDDQPRPRGDNFAVEKPNEN

SEQ ID NO: 109 - DNA, 654 bp, synthetic construct; identical to SEQ ID NO: 66
  misc_feature 1..654: note = DNA sequence of PD Plasmodium berghei ANKA 11-09
  source 1..654: mol_type = other DNA

SEQ ID NO: 110 - protein, 211 aa, synthetic construct; identical to SEQ ID NO: 67
  REGION 1..211: note = Amino acid sequence of PD Plasmodium berghei ANKA 11-09

SEQ ID NO: 111 - DNA, 642 bp, synthetic construct
  misc_feature 1..642: note = DNA sequence of PD Plasmodium falciparum 3D7 11-09
  source 1..642: mol_type = other DNA
ggatccgccaccatggctagcaacctgaagtgcgtgaccaacgtggccggcctggccagcctggtgatcagcctgttccccaagctgatcatcaagaacccccaggtgctgcgccccctgctgaacgtgagctggggctacctgttcggcagcaccttctggctgtgcttcttcagcgaggtgggcctgctgcgcagcctgaagaacatgaagggcgtgcccctgcccgagagcgccagcgaggccaagaagctgctggaggagatgaagaacagcgagggcgacttcaaccgccgcagcctggacttccagtacttcttcagcctggccaccctgttcagcggcatcctgctgctgagcaccgtgaagctggccaaccacaacctgcagctgcgcctgagcagcagcgtggtggtgatcaccagcctgctgaacagcctgtacctgcacaacaaggtgcacaacctgaagagcaagaaggagagcctgtacaacgacttcatcgccaaccccaagaacgagaagaccgtggccgacctgaagaagaacaagaaggagttccacatcttccacggcctgagcgtgctgagcctgtacgtgagcttcttcggcctgaccccctacatcttcacctgactcgag

SEQ ID NO: 112 - protein, 207 aa, synthetic construct
  REGION 1..207: note = Amino acid sequence of PD Plasmodium falciparum 3D7 11-09
MASNLKCVTNVAGLASLVISLFPKLIIKNPQVLRPLLNVSWGYLFGSTFWLCFFSEVGLLRSLKNMKGVPLPESASEAKKLLEEMKNSEGDFNRRSLDFQYFFSLATLFSGILLLSTVKLANHNLQLRLSSSVVVITSLLNSLYLHNKVHNLKSKKESLYNDFIANPKNEKTVADLKKNKKEFHIFHGLSVLSLYVSFFGLTPYIFT

SEQ ID NO: 113 - DNA, 852 bp, synthetic construct
  misc_feature 1..852: note = DNA sequence of Plasmodium berghei ANKA Fusion of PD Pb18-10NT and PD Pb18-10CT
  source 1..852: mol_type = other DNA
atggacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattccctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacag

SEQ ID NO: 114 - protein, 284 aa, synthetic construct
  REGION 1..284: note = Amino acid sequence of Plasmodium berghei ANKA Fusion of PD Pb18-10NT and PD Pb18-10CT
MDNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNSLTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQ

SEQ ID NO: 115 - DNA, 2715 bp, synthetic construct
  misc_feature 1..2715: note = DNA sequence of Plasmodium berghei ANKA Fusion 4cPEAg
  source 1..2715: mol_type = other DNA
ggatccgccaccatggacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattccctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacagctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaacaggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggttggggcgagtatgagccacttgcagtgtctgacatctgttgctggtctgtcctctatcgtcatgtcaatgttccccaaactcattgccaataatccttccctgtttagaccactgctcaacatttcctggggatatctgttcggaagcactgtatggctgtgcttcttcagtgagattgggttggtcaggagaatcaatgctcctaaacggaagaatctgccagagaatgcagaacaagccaaagaacagctgaaggagatcaagaacaacgaaggcgattttaaccgacgcaatatcgacttcaagtacttctttagcctttccacaatcttctctagcatactgctgcttagcacagtgaaactcgccaacaacaatctgcagttgaggatctgttccaccattgtgtcactgagttgcatactgaacaatatgtactttcagaacaagatacactcacttgcactgaagaaagagagtctctttaaggacatgatcgatcgtccgaaagataccactattctggtgaacctgaagaagaacaagaccgactttcacatccatcatggcctttctctgctcttgctctatagcagcttctttggcctcactccctacattttcacgtgactcgag

SEQ ID NO: 116 - protein, 898 aa, synthetic construct
  REGION 1..898: note = Amino acid sequence of Plasmodium berghei ANKA Fusion 4cPEAg
MDNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNSLTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQLIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKKQEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKVGASMSHLQCLTSVAGLSSIVMSMFPKLIANNPSLFRPLLNISWGYLFGSTVWLCFFSEIGLVRRINAPKRKNLPENAEQAKEQLKEIKNNEGDFNRRNIDFKYFFSLSTIFSSILLLSTVKLANNNLQLRICSTIVSLSCILNNMYFQNKIHSLALKKESLFKDMIDRPKDTTILVNLKKNKTDFHIHHGLSLLLLYSSFFGLTPYIFT

SEQ ID NO: 117 - DNA, 3231 bp, synthetic construct
  misc_feature 1..3231: note = DNA sequence of Plasmodium falciparum 3D7 Fusion 4cPEAg
  source 1..3231: mol_type = other DNA
ggatccgccaccatggacaacaacagctacagcttcgagatcgtgaaccgcagcacctggctgaacatcgccgagcgcatcttcaagggcaacgcccccttcaacttcaccatcatcccctacaactacgtgaacaacagcaccgaggagaacaacaacaaggacagcgtgctgctgatcagcaagaacctgaagaacagcagcaaccccgtggacgagaacaaccacatcatcgacagcaccaagaagaacaccagcaacaacaacaacaacaacagcaacatcgtgggcatctacgagagccaggtgcacgaggagaagatcaaggaggacaacacccgccaggacaacatcaacaagaaggagaacgagatcatcaacaacaaccaccagatccccgtgagcaacatcttcagcgagaacatcgacaacaacaagaactacatcgagagcaactacaagagcacctacaacaacaaccccgagctgatccacagcaccgacttcatcggcagcaacaacaaccacaccttcaacttcctgagccgctacaacaacagcgtgctgaacaacatgcagggcaacaccaaggtgcccggcaacgtgcccgagctgaaggcccgcatcttcagcgaggaggagaacaccgaggtggagagcgccgagaacaaccacaccaacagcctgaaccccaacgagagctgcgaccagatcatcaagctgggcgacatcatcaacagcgtgaacgagaagatcatcagcatcaacagcaccgtgaacaacgtgctgtgcatcaacctggacagcgtgaacggcaacggcttcgtgtggaccctgctgggcgtgcacaagaagaagcccctgatcgaccccagcaacttccccaccaagcgcgtgacccagagctacgtgagccccgacatcagcgtgaccaaccccgtgcccatccccaagaacagcaacaccaacaaggacgacagcatcaacaacaagcaggacggcagccagaacaacaccaccaccaaccacttccccaagccccgcgagcagctggtgggcggcagcagcatgctgatcagcaagatcaagccccacaagcccggcaagtacttcatcgtgtacagctactaccgccccttcgaccccacccgcgacaccaacacccgcatcgtggagctgaacgtgcaggaggaggacgacaaggaggtggagcccgaggaggagaaggagaacgagcagaagaaggaggagcaggaggagaacaacctggaggcccccagcaagaccctgatgaagggcgtgaagaccaacatctacttcctgagcaccaaggagcgcatcgaggccctgatgtgctacaactacatcagcaacgccatcatcttcgagaagggcaagttcctgcgctacatcttcatgaacaacgtgaacaacatcatcgtgaacgagcacatgatcaacatgctgtgcaagaaggagaagatcaagtacatcctgagcagcaacagcatcatcatcgagagcaacgacttcatcaagcccctgatcatcgagttcgacagcaacatcagcaagaagatcttcgtgaagcacctgaagatggtggacagcttcaagctggacgacaagctgtaccgcgagtacctgaacgacctgagcgagcacgagcgcgaccgcctgaagcacgtggagagcttctacagcaacgccatcaaggtgcacaacgaggacgtgcagaacaacatcgtggacgagatcaagtaccgcgaggaggtgtgcaacgacgaggtggacctgtacctgctgatggactgcagcggcagcatccgccgccacaactgggtgaaccacgccgtgcccctggccatgaagctgatccagcagctgaacctgaacgacaacgccatccacctgtacgccagcgtgttcagcaacaacgcccgcgagatcatccgcctgcacagcgacgccagcaagaacaaggagaaggccctgatcatcatcaagagcctgctgagcaccaacctgccctacggcaagaccaacctgaccgacgccctgctgcaggtgcgcaagcacctgaacgaccgcatcaaccgcgagaacgccaaccagctggtggtgatcctgaccgacggcatccccgacagcatccaggacagcctgaaggagagccgcaagctgagcgaccgcggcgtgaagatcgccgtgttcggcatcggccagggcatcaacgtggccttcaaccgcttcctggtgggctgccaccccagcgacggcaagtgcaacctgtacgccgacagcgcctgggagaacgtgaagaacgtgatcggccccttcatgaaggccgtgtgcgtggaggtggagaagaccgccagctgcggcgtgtgggacgagtggagcccctgcagcgtgacctgcggcaagggcacccgcagccgcaagcgcgagatcctgcacgagggctgcaccagcgagctgcaggagcagtgcgaggaggagcgctgcctgcccaagcgcgagcccctggacgtgcccgacgagcccgaggacgaccagccccgcccccgcggcgacaacttcgccgtggagaagcccaacgagaacagcaacctgaagtgcgtgaccaacgtggccggcctggccagcctggtgatcagcctgttccccaagctgatcatcaagaacccccaggtgctgcgccccctgctgaacgtgagctggggctacctgttcggcagcaccttctggctgtgcttcttcagcgaggtgggcctgctgcgcagcctgaagaacatgaagggcgtgcccctgcccgagagcgccagcgaggccaagaagctgctggaggagatgaagaacagcgagggcgacttcaaccgccgcagcctggacttccagtacttcttcagcctggccaccctgttcagcggcatcctgctgctgagcaccgtgaagctggccaaccacaacctgcagctgcgcctgagcagcagcgtggtggtgatcaccagcctgctgaacagcctgtacctgcacaacaaggtgcacaacctgaagagcaagaaggagagcctgtacaacgacttcatcgccaaccccaagaacgagaagaccgtggccgacctgaagaagaacaagaaggagttccacatcttccacggcctgagcgtgctgagcctgtacgtgagcttcttcggcctgaccccctacatcttcacctgactcgag

SEQ ID NO: 118 - protein, 1070 aa, synthetic construct
  REGION 1..1070: note = Amino acid sequence of Plasmodium falciparum 3D7 Fusion 4cPEAg
MDNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQEEDDKEVEPEEEKENEQKKEEQEENNLEAPSKTLMKGVKTNIYFLSTKERIEALMCYNYISNAIIFEKGKFLRYIFMNNVNNIIVNEHMINMLCKKEKIKYILSSNSIIIESNDFIKPLIIEFDSNISKKIFVKHLKMVDSFKLDDKLYREYLNDLSEHERDRLKHVESFYSNAIKVHNEDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCLPKREPLDVPDEPEDDQPRPRGDNFAVEKPNENSNLKCVTNVAGLASLVISLFPKLIIKNPQVLRPLLNVSWGYLFGSTFWLCFFSEVGLLRSLKNMKGVPLPESASEAKKLLEEMKNSEGDFNRRSLDFQYFFSLATLFSGILLLSTVKLANHNLQLRLSSSVVVITSLLNSLYLHNKVHNLKSKKESLYNDFIANPKNEKTVADLKKNKKEFHIFHGLSVLSLYVSFFGLTPYIFT

SEQ ID NO: 119 - DNA, 3597 bp, synthetic construct
  misc_feature 1..3597: note = DNA sequence of Plasmodium berghei Fusion 5cPEAg
  source 1..3597: mol_type = other DNA
ggatccgccaccatggcccagaacaaatccattcaggcacagaggaacctcaacgaactctgctacaatgaagggaatgacaataagctgtatcacgtgctgaattccaagaacggcaaaatctacaatcgcaacacagtaaatcggttgcttgccgatgcacccgagggtaagaagaatgaaaagaagaatgagaaaatcgagcgcaacaacaagcttaaacagccaccgcctcctcctaacccaaatgacccaccgccacccaatccaaacgacccaccgcctcccaaccctaacgatcctccaccgcccaaccctaatgacccaccacctcccaatgcaaacgatccaccccctcctaacgctaacgaccctgctccacccaacgctaacgatcccgcgccccccaatgccaacgaccccgcaccacctaatgccaacgatcccgccccgcccaatgctaatgatcctccaccacccaacccaaacgaccctgcccctcctaatgctaacgatccaccacctcccaatccgaatgatcccgctccacctcaggggaacaacaaccctcagccccaacctagaccacagccgcagccccaaccccaaccccagccccagcctcaaccccagccccagccacgtccgcagcctcagcctcaacctggaggcaacaataacaacaagaataacaacaacgacgacagctacattccaagtgccgagaaaattctggagtttgttaagcagatccgagacagcataaccgaagaatggtcacagtgtaacgtgacgtgtggatctggcatcagagtgaggaaacggaagggttccaataagaaagcagaggatctgactctggaggacattgatacagagatctgcaaaatggacaaagacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattccctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacagctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaacaggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggttggggcgagtatgagccacttgcagtgtctgacatctgttgctggtctgtcctctatcgtcatgtcaatgttccccaaactcattgccaataatccttccctgtttagaccactgctcaacatttcctggggatatctgttcggaagcactgtatggctgtgcttcttcagtgagattgggttggtcaggagaatcaatgctcctaaacggaagaatctgccagagaatgcagaacaagccaaagaacagctgaaggagatcaagaacaacgaaggcgattttaaccgacgcaatatcgacttcaagtacttctttagcctttccacaatcttctctagcatactgctgcttagcacagtgaaactcgccaacaacaatctgcagttgaggatctgttccaccattgtgtcactgagttgcatactgaacaatatgtactttcagaacaagatacactcacttgcactgaagaaagagagtctctttaaggacatgatcgatcgtccgaaagataccactattctggtgaacctgaagaagaacaagaccgactttcacatccatcatggcctttctctgctcttgctctatagcagcttctttggcctcactccctacattttcacgtgactcgag

SEQ ID NO: 120 - protein, 1192 aa, synthetic construct
  REGION 1..1192: note = Amino acid sequence of Plasmodium berghei Fusion 5cPEAg SP
MAQNKSIQAQRNLNELCYNEGNDNKLYHVLNSKNGKIYNRNTVNRLLADAPEGKKNEKKNEKIERNNKLKQPPPPPNPNDPPPPNPNDPPPPNPNDPPPPNPNDPPPPNANDPPPPNANDPAPPNANDPAPPNANDPAPPNANDPAPPNANDPPPPNPNDPAPPNANDPPPPNPNDPAPPQGNNNPQPQPRPQPQPQPQPQPQPQPQPQPRPQPQPQPGGNNNNKNNNNDDSYIPSAEKILEFVKQIRDSITEEWSQCNVTCGSGIRVRKRKGSNKKAEDLTLEDIDTEICKMDKDNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNSLTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQLIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKKQEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKVGASMSHLQCLTSVAGLSSIVMSMFPKLIANNPSLFRPLLNISWGYLFGSTVWLCFFSEIGLVRRINAPKRKNLPENAEQAKEQLKEIKNNEGDFNRRNIDFKYFFSLSTIFSSILLLSTVKLANNNLQLRICSTIVSLSCILNNMYFQNKIHSLALKKESLFKDMIDRPKDTTILVNLKKNKTDFHIHHGLSLLLLYSSFFGLTPYIFT

SEQ ID NO: 121 - DNA, 3663 bp, synthetic construct
  misc_feature 1..3663: note = DNA sequence of Plasmodium berghei Fusion 5cPEAg SP+
  source 1..3663: mol_type = other DNA
ggatccgccaccatggccaagaagtgtaccatactggtcgttgcgtcacttctgttggtcaattctctgctcccaggctatggacagaacaaatccattcaggcacagaggaacctcaacgaactctgctacaatgaagggaatgacaataagctgtatcacgtgctgaattccaagaacggcaaaatctacaatcgcaacacagtaaatcggttgcttgccgatgcacccgagggtaagaagaatgaaaagaagaatgagaaaatcgagcgcaacaacaagcttaaacagccaccgcctcctcctaacccaaatgacccaccgccacccaatccaaacgacccaccgcctcccaaccctaacgatcctccaccgcccaaccctaatgacccaccacctcccaatgcaaacgatccaccccctcctaacgctaacgaccctgctccacccaacgctaacgatcccgcgccccccaatgccaacgaccccgcaccacctaatgccaacgatcccgccccgcccaatgctaatgatcctccaccacccaacccaaacgaccctgcccctcctaatgctaacgatccaccacctcccaatccgaatgatcccgctccacctcaggggaacaacaaccctcagccccaacctagaccacagccgcagccccaaccccaaccccagccccagcctcaaccccagccccagccacgtccgcagcctcagcctcaacctggaggcaacaataacaacaagaataacaacaacgacgacagctacattccaagtgccgagaaaattctggagtttgttaagcagatccgagacagcataaccgaagaatggtcacagtgtaacgtgacgtgtggatctggcatcagagtgaggaaacggaagggttccaataagaaagcagaggatctgactctggaggacattgatacagagatctgcaaaatggacaaagacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattccctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacagctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaacaggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggttggggcgagtatgagccacttgcagtgtctgacatctgttgctggtctgtcctctatcgtcatgtcaatgttccccaaactcattgccaataatccttccctgtttagaccactgctcaacatttcctggggatatctgttcggaagcactgtatggctgtgcttcttcagtgagattgggttggtcaggagaatcaatgctcctaaacggaagaatctgccagagaatgcagaacaagccaaagaacagctgaaggagatcaagaacaacgaaggcgattttaaccgacgcaatatcgacttcaagtacttctttagcctttccacaatcttctctagcatactgctgcttagcacagtgaaactcgccaacaacaatctgcagttgaggatctgttccaccattgtgtcactgagttgcatactgaacaatatgtactttcagaacaagatacactcacttgcactgaagaaagagagtctctttaaggacatgatcgatcgtccgaaagataccactattctggtgaacctgaagaagaacaagaccgactttcacatccatcatggcctttctctgctcttgctctatagcagcttctttggcctcactccctacattttcacgtgactcgag

SEQ ID NO: 122 - protein, 1214 aa, synthetic construct
  REGION 1..1214: note = Amino acid sequence of Plasmodium berghei Fusion 5cPEAg SP+
MAKKCTILVVASLLLVNSLLPGYGQNKSIQAQRNLNELCYNEGNDNKLYHVLNSKNGKIYNRNTVNRLLADAPEGKKNEKKNEKIERNNKLKQPPPPPNPNDPPPPNPNDPPPPNPNDPPPPNPNDPPPPNANDPPPPNANDPAPPNANDPAPPNANDPAPPNANDPAPPNANDPPPPNPNDPAPPNANDPPPPNPNDPAPPQGNNNPQPQPRPQPQPQPQPQPQPQPQPQPRPQPQPQPGGNNNNKNNNNDDSYIPSAEKILEFVKQIRDSITEEWSQCNVTCGSGIRVRKRKGSNKKAEDLTLEDIDTEICKMDKDNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNSLTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQLIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKKQEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKVGASMSHLQCLTSVAGLSSIVMSMFPKLIANNPSLFRPLLNISWGYLFGSTVWLCFFSEIGLVRRINAPKRKNLPENAEQAKEQLKEIKNNEGDFNRRNIDFKYFFSLSTIFSSILLLSTVKLANNNLQLRICSTIVSLSCILNNMYFQNKIHSLALKKESLFKDMIDRPKDTTILVNLKKNKTDFHIHHGLSLLLLYSSFFGLTPYIFT

SEQ ID NO: 123 - DNA, 1044 bp, synthetic construct
  misc_feature 1..1044: note = DNA sequence of Plasmodium berghei ANKA CSP
  source 1..1044: mol_type = other DNA
ggatccgccaccatggccaagaagtgtaccatactggtcgttgcgtcacttctgttggtcaattctctgctcccaggctatggacagaacaaatccattcaggcacagaggaacctcaacgaactctgctacaatgaagggaatgacaataagctgtatcacgtgctgaattccaagaacggcaaaatctacaatcgcaacacagtaaatcggttgcttgccgatgcacccgagggtaagaagaatgaaaagaagaatgagaaaatcgagcgcaacaacaagcttaaacagccaccgcctcctcctaacccaaatgacccaccgccacccaatccaaacgacccaccgcctcccaaccctaacgatcctccaccgcccaaccctaatgacccaccacctcccaatgcaaacgatccaccccctcctaacgctaacgaccctgctccacccaacgctaacgatcccgcgccccccaatgccaacgaccccgcaccacctaatgccaacgatcccgccccgcccaatgctaatgatcctccaccacccaacccaaacgaccctgcccctcctaatgctaacgatccaccacctcccaatccgaatgatcccgctccacctcaggggaacaacaaccctcagccccaacctagaccacagccgcagccccaaccccaaccccagccccagcctcaaccccagccccagccacgtccgcagcctcagcctcaacctggaggcaacaataacaacaagaataacaacaacgacgacagctacattccaagtgccgagaaaattctggagtttgttaagcagatccgagacagcataaccgaagaatggtcacagtgtaacgtgacgtgtggatctggcatcagagtgaggaaacggaagggttccaataagaaagcagaggatctgactctggaggacattgatacagagatctgcaaaatggacaaatgcagctctatcttcaacatcgtgagtaatagcctcgggtttgtgattctgctggtcctggtgttcttcaattgactcgag

SEQ ID NO: 124 - protein, 341 aa, synthetic construct; identical to SEQ ID NO: 11
  REGION 1..341: note = Amino acid sequence of Plasmodium berghei ANKA CSP